(19) United States
(12) Patent Application Publication
(10) Pub. No.: US 2024/0228475 A1
(43) Pub. Date: Jul. 11, 2024

(54) PROCESSES FOR THE PREPARATION OF (S)-2-(2,6-DIOXOPIPERIDIN-3-YL)-4-((2-FLUORO-4-((3-MORPHOLINOAZETIDIN-1-YL)METHYL)BENZYL)AMINO)ISOINDOLINE-1,3-DIONE

(71) Applicant: Celgene Corporation, Summit, NJ (US)

(72) Inventors: Ronald CARRASQUILLO-FLORES, Somerset, NJ (US); Jian CHEN, Hillsborough, NJ (US); Patrick CORONA, Jersey City, NJ (US); David DEL VALLE, Scotch Plains, NJ (US); Robert Francis DUNN, Towaco, NJ (US); Megan EMMANUEL, Plainsboro, NJ (US); Antonio C. FERRETTI, Summit, NJ (US); Richard Martin HEID, Maplewood, NJ (US); Amude KASSIM, Fairless Hills, PA (US); Mohit KOTHARE, Bridgewater, NJ (US); Wei LIU, Green Brook, NJ (US); Geoffrey Eugene PURDUM, Freehold, NJ (US); Krishnakumar RANGANATHAN, Malvern, PA (US); Paula A. TAVARES-GRECO, Parsippany, NJ (US); Kelvin Hin-Yeong YONG, Westfield, NJ (US); Yong YU, Oro Valley, AZ (US); Chengmin ZHANG, Florham Park, NJ (US)

(21) Appl. No.: 18/572,342

(22) PCT Filed: Jun. 17, 2022
(86) PCT No.: PCT/US2022/034028

§ 371 Date: Dec. 20, 2023

Related U.S. Application Data
(60) Provisional application No. 63/213,043, filed on Jun. 21, 2021.

(51) Int. Cl. C07D 413/14

(52) U.S. Cl. CPC C07D 413/14

(57) ABSTRACT

Provided herein are processes for the preparation of (S)-2-(2,6-dioxopipelidin-3-yl)-4-((2-fluoro-4-((3-morpholinoazetidin-1-yl) methyl) benzyl)amino)isoindoline-1, 3-dione, or a salt, solvate, hydrate, enantiomer, mixture of enantiomers, or isotopologue thereof, which is useful for treating, preventing, and managing various disorders. Also provided are solid forms of various intermediates and products obtained from the processes.

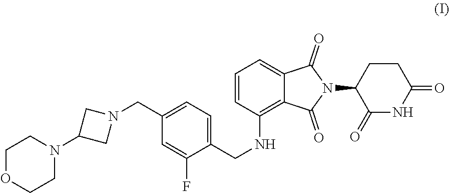 [SMILES: O=C1CC[C@H](N2C(=O)c3cccc(NCc4ccc(CN5CC(N6CCOCC6)C5)cc4F)c3C2=O)C(=O)N1]

### 1. CROSS-REFERENCE TO RELATED APPLICATIONS

This application claims the benefit of priority to U.S. Ser. No. 63/213,043, filed Jun. 21, 2021, which is incorporated herein by reference in its entirety.

### 2. FIELD

Provided herein are processes for the preparation of (S)-2-(2,6-dioxopiperidin-3-yl)-4-((2-fluoro-4-((3-morpholinoazetidin-1-yl)methyl)benzyl)amino)isoindoline-1,3-dione, or a salt, solvate, hydrate, enantiomer, mixture of enantiomers, or isotopologue thereof, which is useful for treating, preventing, and managing various disorders.

### 3. BACKGROUND

Cancer is characterized primarily by an increase in the number of abnormal cells derived from a given normal tissue, invasion of adjacent tissues by these abnormal cells, or lymphatic or blood-borne spread of malignant cells to regional lymph nodes and metastasis. Clinical data and molecular biologic studies indicate that cancer is a multistep process that begins with minor preneoplastic changes, which may under certain conditions progress to neoplasia. The neoplastic lesion may evolve clonally and develop an increasing capacity for invasion, growth, metastasis, and heterogeneity, especially under conditions in which the neoplastic cells escape the host's immune surveillance. Current cancer therapy may involve surgery, chemotherapy, hormonal therapy and/or radiation treatment to eradicate neoplastic cells in a patient. Recent advances in cancer therapeutics are discussed by Rajkumar et al. in Nature Reviews Clinical Oncology 11, 628-630 (2014).

Hematological malignancies are cancers that begin in blood-forming tissue, such as the bone marrow, or in the cells of the immune system. Examples of hematological malignancies are leukemia, lymphoma, and myeloma. More specific examples of hematological malignancies include but are not limited to acute myeloid leukemia (AML), acute lymphocytic leukemia (ALL), multiple myeloma (MM), non-Hodgkin's lymphoma (NHL), diffuse large B-cell lymphoma (DLBCL), Hodgkin's lymphoma (HL), T-cell lymphoma (TCL), Burkitt lymphoma (BL), chronic lymphocytic leukemia/small lymphocytic lymphoma (CLL/SLL), marginal zone lymphoma (MZL), and myelodysplastic syndromes (MDS).

Certain 4-aminoisoindoline-1,3-dione compounds, including (S)-2-(2,6-dioxopiperidin-3-yl)-4-((2-fluoro-4-((3-morpholinoazetidin-1-yl)methyl)benzyl)amino)isoindoline-1,3-dione, have been reported to be effective against various hematological cancer cell lines. See U.S. Patent Publication Nos. 2019/0322647 and 2020/0325129, each of which is incorporated herein by reference in its entirety.

Methods for synthesizing (S)-2-(2,6-dioxopiperidin-3-yl)-4-((2-fluoro-4-((3-morpholinoazetidin-1-yl)methyl)benzyl)amino)isoindoline-1,3-dione and its racemic compound have been previously described in U.S. Patent Publication No. 2019/0322647. A need still exists for efficient and scalable processes for the preparation of (S)-2-(2,6-dioxopiperidin-3-yl)-4-((2-fluoro-4-((3-morpholinoazetidin-1-yl)methyl)benzyl)amino)isoindoline-1,3-dione, or a salt, solvate, hydrate, enantiomer, mixture of enantiomers, or isotopologue thereof.

### 4. SUMMARY

In one embodiment, provided herein is a process for preparing a compound of Formula (I):

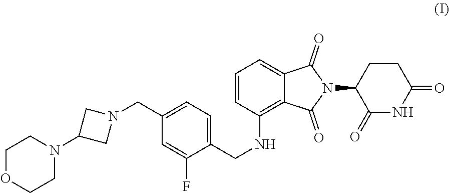 [SMILES: O=C1CC[C@H](N2C(=O)c3cccc(NCc4ccc(CN5CC(N6CCOCC6)C5)cc4F)c3C2=O)C(=O)N1]

or a salt, solvate, hydrate, enantiomer, mixture of enantiomers, or isotopologue thereof, comprising:
(step 1.0) cyclizing a compound of Formula (II):

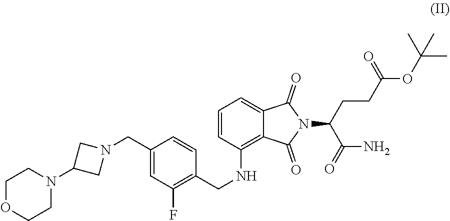 [SMILES: CC(C)(C)OC(=O)CC[C@@H](C(N)=O)N1C(=O)c2cccc(NCc3ccc(CN4CC(N5CCOCC5)C4)cc3F)c2C1=O]

or a salt, solvate, hydrate, enantiomer, mixture of enantiomers, or isotopologue thereof, to provide a compound of Formula (I), or a salt, solvate, hydrate, enantiomer, mixture of enantiomers, or isotopologue thereof; and
(step 1.1) optionally converting the compound of Formula (I), or a salt, solvate, hydrate, enantiomer, mixture of enantiomers, or isotopologue thereof, to a salt of the compound.

In one embodiment, provided herein are solid forms (e.g., Form B) comprising a besylate salt of Compound 1:

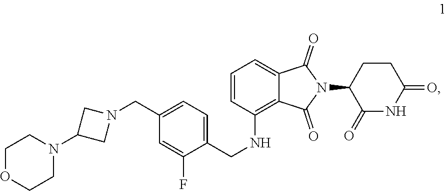 [SMILES: O=C1CC[C@H](N2C(=O)c3cccc(NCc4ccc(CN5CC(N6CCOCC6)C5)cc4F)c3C2=O)C(=O)N1]

In one embodiment, provided herein are solid forms (e.g., Form A or Form B) comprising a hydrochloride salt of Compound 3:

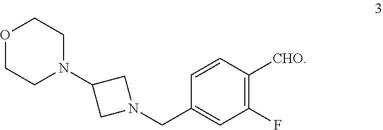 [SMILES: O=Cc1ccc(CN2CC(N3CCOCC3)C2)cc1F]

In one embodiment, provided herein are solid forms (e.g., Form A) comprising a methanesulfonic acid salt of Compound 4:

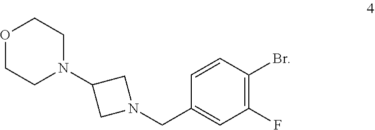 [SMILES: Fc1cc(CN2CC(N3CCOCC3)C2)ccc1Br]

### 5. BRIEF DESCRIPTION OF THE FIGURES

FIG. 1 provides a representative XRPD pattern of Form B of a besylate salt of Compound 1.

FIG. 2 provides a representative TGA thermogram of Form B of a besylate salt of Compound 1.

FIG. 3 provides a representative DSC thermogram of Form B of a besylate salt of Compound 1.

FIG. 4 provides a representative XRPD pattern of Form A of a hydrochloride salt of Compound 1 (a) produced according the methods described herein in comparison to an reference sample (b).

FIG. 5 provides a representative XRPD pattern of Form A of a hydrochloride salt of Compound 3.

FIG. 6 provides a representative DSC thermogram of Form A of a hydrochloride salt of Compound 3.

FIG. 7 provides a representative XRPD pattern of Form B of a hydrochloride salt of Compound 3.

FIG. 8 provides a representative TGA thermogram of Form B of a hydrochloride salt of Compound 3.

FIG. 9 provides a representative DSC thermogram of Form B of a hydrochloride salt of Compound 3.

FIG. 10 provides a representative XRPD pattern of Form A of a methanesulfonic acid salt of Compound 4.

FIG. 11 provides a representative DSC thermogram of Form A of a methanesulfonic acid salt of Compound 4.

### 6. DETAILED DESCRIPTION

### 6.1 Definition

As used herein and unless otherwise indicated, the term “process(es)” provided herein refers to the methods provided herein which are useful for preparing a compound provided herein. Modifications to the methods provided herein (e.g., starting materials, reagents, protecting groups, solvents, temperatures, reaction times, purification) are also encompassed by the present disclosure. In general, the technical teaching of one embodiment provided herein can be combined with that disclosed in any other embodiments provided herein.

The use of the word “a” or “an” when used in conjunction with the term “comprising” in the claims and/or the specification can mean “one”, but it is also consistent with the meaning of “one or more”, “at least one” and “one or more than one.”

As used herein, the terms “comprising” and “including” can be used interchangeably. The terms “comprising” and “including” are to be interpreted as specifying the presence of the stated features or components as referred to, but does not preclude the presence or addition of one or more features, or components, or groups thereof. Additionally, the terms “comprising” and “including” are intended to include examples encompassed by the term “consisting of”. Consequently, the term “consisting of” can be used in place of the terms “comprising” and “including” to provide for more specific embodiments of the invention.

The term “consisting of” means that a subject-matter has at least 90%, 95%, 97%, 98% or 99% of the stated features or components of which it consists. In another embodiment the term “consisting of” excludes from the scope of any succeeding recitation any other features or components, excepting those that are not essential to the technical effect to be achieved.

As used herein, the terms “or” is to be interpreted as an inclusive “or” meaning any one or any combination. Therefore, “A, B or C” means any of the following: “A; B; C; A and B; A and C; B and C; A, B and C”. An exception to this definition will occur only when a combination of elements, functions, steps or acts are in some way inherently mutually exclusive.

As used herein, and unless otherwise indicated, the term “adding,” “reacting,” “treating,” or the like means contacting one reactant, reagent, solvent, catalyst, reactive group or the like with another reactant, reagent, solvent, catalyst, reactive group or the like. Reactants, reagents, solvents, catalysts, reactive group or the like can be added individually, simultaneously or separately and can be added in any order. Reactants, reagents, solvents, catalysts, reactive group or the like can each respectively be added in one portion, which may be delivered all at once or over a period of time, or in discrete portions, which also may be delivered all at once or over a period of time. They can be added in the presence or absence of heat and can optionally be added under an inert atmosphere. “Reacting” can refer to in situ formation or intramolecular reaction where the reactive groups are in the same molecule.

As used herein, and unless otherwise indicated, the term “transforming” refers to subjecting the compound at hand to reaction conditions suitable to effect the formation of the desired compound at hand.

As used herein, and unless otherwise indicated, the term “salt” includes, but is not limited to, salts of acidic or basic groups that may be present in the compounds provided herein. Compounds that are basic in nature are capable of forming a wide variety of salts with various inorganic and organic acids. The acids that may be used to prepare salts of such basic compounds are those that form salts comprising anions including, but not limited to, acetate, benzenesulfonate, benzoate, bicarbonate, bitartrate, bromide, calcium edetate, camsylate, carbonate, chloride, bromide, iodide, citrate, dihydrochloride, edetate, edisylate, estolate, esylate, fumarate, gluceptate, gluconate, glutamate, glycollylarsanilate, hexylresorcinate, hydrabamine, hydroxynaphthoate, isethionate, lactate, lactobionate, malate, maleate, mandelate, mesylate, methylsulfate, muscate, napsylate, nitrate, panthothenate, phosphate/diphosphate, polygalacturonate, salicylate, stearate, succinate, sulfate, tannate, tartrate, teoclate, triethiodide, and pamoate. Compounds that include an amino group also can form salts with various amino acids, in addition to the acids mentioned above. Compounds that are acidic in nature are capable of forming base salts with various cations. Non-limiting examples of such salts include alkali metal or alkaline earth metal salts and, in some embodiments, calcium, magnesium, sodium, lithium, zinc, potassium, and iron salts. Compounds that are acidic in nature are also capable of forming base salts with compounds that include an amino group.

As used herein, and unless otherwise specified, the term “solvate” means a compound that further includes a stoichiometric or non-stoichiometric amount of solvent bound by non-covalent intermolecular forces. Where the solvent is water, the solvate is a hydrate.

As used herein, and unless otherwise specified, the term “stereoisomer” encompasses all enantiomerically/stereomerically pure and enantiomerically/stereomerically enriched compounds provided herein.

If the stereochemistry of a structure or a portion thereof is not indicated, e.g., with bold or dashed lines, the structure or portion thereof is to be interpreted as encompassing all enantiomerically pure, enantiomerically enriched, diastereomerically pure, diastereomerically enriched, and racemic mixtures of the compounds.

Unless otherwise indicated, the terms “enantiomerically enriched” and “enantiomerically pure,” as used interchangeably herein, refer to compositions in which the percent by weight of one enantiomer is greater than the amount of that one enantiomer in a control mixture of the racemic composition (e.g., greater than 1:1 by weight). For example, an enantiomerically enriched preparation of the (S)-enantiomer, means a preparation of the compound having greater than 50% by weight of the (S)-enantiomer relative to the (R)-enantiomer, such as at least 75% by weight, and even such as at least 80% by weight. In some embodiments, the enrichment can be much greater than 80% by weight, providing a “substantially optically enriched,” “substantially enantiomerically enriched,” “substantially enantiomerically pure” or a “substantially non-racemic” preparation, which refers to preparations of compositions which have at least 85% by weight of one enantiomer relative to other enantiomer, such as at least 90% by weight, and such as at least 95% by weight. In one embodiment, the compositions have about 99% by weight of one enantiomer relative to other enantiomer. In one embodiment, the compositions have greater than at least 99% by weight of one enantiomer relative to other enantiomer. In some embodiments, the enantiomerically enriched composition has a higher potency with respect to therapeutic utility per unit mass than does the racemic mixture of that composition.

As used herein and unless otherwise specified, the terms “solid form” and related terms refer to a physical form which is not predominantly in a liquid or a gaseous state. As used herein, the terms “solid form” and “solid forms” encompass semi-solids. Solid forms may be crystalline, amorphous, partially crystalline, partially amorphous, or mixtures of forms.

The solid forms provided herein may have varying degrees of crystallinity or lattice order. The solid forms provided herein are not limited by any particular degree of crystallinity or lattice order, and may be 0-100% crystalline. Methods of determining the degree of crystallinity are known to those of ordinary skill in the, such as those described in Suryanarayanan, R., X-Ray Power Diffractometry, Physical Characterization of Pharmaceutical Salts, H. G. Brittain, Editor, Mercel Dekkter, Murray Hill, N.J., 1995, pp. 187-199, which is incorporated herein by reference in its entirety. In some embodiments, the solid forms provided herein are about 0, 5, 10, 15, 20, 25, 30, 35, 40, 45, 50, 55, 60, 65, 70, 75, 80, 85, 90, 95 or 100% crystalline.

As used herein and unless otherwise specified, the term “crystalline” and related terms used herein, when used to describe a substance, component, product, or form, mean that the substance, component, product, or form is substantially crystalline, for example, as determined by X-ray diffraction. See, e.g., Remington: The Science and Practice of Pharmacy, 21^{st} edition, Lippincott, Williams and Wilkins, Baltimore, M D (2005); The United States Pharmacopeia, 23^{rd} edition, 1843-1844 (1995).

As used herein and unless otherwise specified, the term “crystal form,” “crystal forms,” and related terms herein refer to solid forms that are crystalline. Crystal forms include single-component crystal forms and multiple-component crystal forms, and include, but are not limited to, polymorphs, solvates, hydrates, and other molecular complexes, as well as salts, solvates of salts, hydrates of salts, co-crystals of salts, other molecular complexes of salts, and polymorphs thereof. In certain embodiments, a crystal form of a substance may be substantially free of amorphous forms and/or other crystal forms. In certain embodiments, a crystal form of a substance may contain less than about 1%, 2%, 3%, 4%, 5%, 6%, 7%, 8%, 9%, 10%, 15%, 20%, 25%, 30%, 35%, 40%, 45% or 50% of one or more amorphous form(s) and/or other crystal form(s) on a weight basis. In certain embodiments, a crystal form of a substance may be physically and/or chemically pure. In certain embodiments, a crystal form of a substance may be about 99%, 98%, 97%, 96%, 95%, 94%, 93%, 92%, 91% or 90% physically and/or chemically pure.

Crystal forms of a substance may be obtained by a number of methods. Such methods include, but are not limited to, melt recrystallization, melt cooling, solvent recrystallization, recrystallization in confined spaces such as, e.g., in nanopores or capillaries, recrystallization on surfaces or templates such as, e.g., on polymers, recrystallization in the presence of additives, such as, e.g., co-crystal counter-molecules, desolvation, dehydration, rapid evaporation, rapid cooling, slow cooling, vapor diffusion, sublimation, grinding, and solvent-drop grinding.

Unless otherwise specified, the terms “polymorph,” “polymorphic form,” “polymorphs,” “polymorphic forms,” and related terms herein refer to two or more crystal forms that consist essentially of the same molecule, molecules or ions. Like different crystal forms, different polymorphs may have different physical properties, such as, for example, melting temperatures, heats of fusion, solubilities, dissolution rates, and/or vibrational spectra as a result of a different arrangement or conformation of the molecules or ions in the crystal lattice. The differences in physical properties exhibited by polymorphs may affect pharmaceutical parameters, such as storage stability, compressibility and density (important in formulation and product manufacturing), and dissolution rate (an important factor in bioavailability). Differences in stability can result from changes in chemical reactivity (e.g., differential oxidation, such that a dosage form discolors more rapidly when comprised of one polymorph than when comprised of another polymorph) or mechanical changes (e.g., tablets crumble on storage as a kinetically favored polymorph converts to thermodynamically a more stable polymorph) or both (e.g., tablets of one polymorph are more susceptible to breakdown at high humidity). As a result of solubility/dissolution differences, in the extreme case, some polymorphic transitions may result in lack of potency or, at the other extreme, toxicity. In addition, the physical properties of the crystal may be important in processing (for example, one polymorph might be more likely to form solvates or might be difficult to filter and wash free of impurities, and particle shape and size distribution might be different between polymorphs).

As used herein and unless otherwise specified, the term “amorphous,” “amorphous form,” and related terms used herein, mean that the substance, component or product in question is not substantially crystalline as determined by X-ray diffraction. In particular, the term “amorphous form” describes a disordered solid form, i.e., a solid form lacking long range crystalline order. In certain embodiments, an amorphous form of a substance may be substantially free of other amorphous forms and/or crystal forms. In other embodiments, an amorphous form of a substance may contain less than about 1%, 2%, 3%, 4%, 5%, 10%, 15%, 20%, 25%, 30%, 35%, 40%, 45% or 50% of one or more other amorphous forms and/or crystal forms on a weight basis. In certain embodiments, an amorphous form of a substance may be physically and/or chemically pure. In certain embodiments, an amorphous form of a substance may be about 99%, 98%, 97%, 96%, 95%, 94%, 93%, 92%, 91% or 90% physically and/or chemically pure. In certain embodiments, an amorphous form of a substance may comprise additional components or ingredients (for example, an additive, a polymer, or an excipient that may serve to further stabilize the amorphous form). In certain embodiments, amorphous form may be a solid solution.

Amorphous forms of a substance can be obtained by a number of methods. Such methods include, but are not limited to, heating, melt cooling, rapid melt cooling, solvent evaporation, rapid solvent evaporation, desolvation, sublimation, grinding, ball-milling, cryo-grinding, spray drying, and freeze drying.

Techniques for characterizing crystal forms and amorphous forms include, but are not limited to, thermal gravimetric analysis (TGA), differential scanning calorimetry (DSC), X-ray powder diffractometry (XRPD), single-crystal X-ray diffractometry, vibrational spectroscopy, e.g., infrared (IR) and Raman spectroscopy, solid-state and solution nuclear magnetic resonance (NMR) spectroscopy, optical microscopy, hot stage optical microscopy, scanning electron microscopy (SEM), electron crystallography and quantitative analysis, particle size analysis (PSA), surface area analysis, solubility measurements, dissolution measurements, elemental analysis and Karl Fischer analysis. Characteristic unit cell parameters may be determined using one or more techniques such as, but not limited to, X-ray diffraction and neutron diffraction, including single-crystal diffraction and powder diffraction. Techniques useful for analyzing powder diffraction data include profile refinement, such as Rietveld refinement, which may be used, e.g., to analyze diffraction peaks associated with a single phase in a sample comprising more than one solid phase. Other methods useful for analyzing powder diffraction data include unit cell indexing, which allows one of skill in the art to determine unit cell parameters from a sample comprising crystalline powder.

Solid forms may exhibit distinct physical characterization data that are unique to a particular solid form, such as the crystal forms provided herein. These characterization data may be obtained by various techniques known to those skilled in the art, including for example X-ray powder diffraction, differential scanning calorimetry, thermal gravimetric analysis, and nuclear magnetic resonance spectroscopy. The data provided by these techniques may be used to identify a particular solid form. One skilled in the art can determine whether a solid form is one of the forms provided herein by performing one of these characterization techniques and determining whether the resulting data “matches” the reference data provided herein, which is identified as being characteristic of a particular solid form. Characterization data that “matches” those of a reference solid form is understood by those skilled in the art to correspond to the same solid form as the reference solid form. In analyzing whether data “match,” a person of ordinary skill in the art understands that particular characterization data points may vary to a reasonable extent while still describing a given solid form, due to, for example, experimental error and routine sample-to-sample analysis variation.

As used herein, and unless otherwise indicated, the term “halo”, “halogen”, or the like means —F, —Cl, —Br, or —I.

As used herein, and unless otherwise indicated, the term “alkyl” means a saturated, monovalent, unbranched or branched hydrocarbon chain. Examples of alkyl groups include, but are not limited to, (C₁-C₆)alkyl groups, such as methyl, ethyl, propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, butyl, isobutyl, t-butyl, pentyl, isopentyl, neopentyl, and hexyl. Longer alkyl groups include heptyl, octyl, nonyl and decyl groups. An alkyl group can be unsubstituted or substituted with one or more suitable substituents. The alkyl groups may also be isotopologues of the natural abundance alkyl groups by being enriched in isotopes of carbon and/or hydrogen (i.e., deuterium or tritium). As used herein, and unless otherwise indicated, the term “alkenyl” means an unbranched or branched monovalent hydrocarbon chain, which contains one or more carbon-carbon double bonds. As used herein, and unless otherwise indicated, the term “alkynyl” means an unbranched or branched monovalent hydrocarbon chain, which contains one or more carbon-carbon triple bonds.

As used herein, and unless otherwise indicated, the term “alkoxy” means an alkyl group that is linked to another group via an oxygen atom (i.e., —O-alkyl). An alkoxy group can be unsubstituted or substituted with one or more suitable substituents. Examples of alkoxy groups include, but are not limited to, (C₁-C₆)alkoxy groups, such as —O-methyl, —O-ethyl, —O-propyl, —O-isopropyl, —O-2-methyl-1-propyl, —O-2-methyl-2-propyl, —O-2-methyl-1-butyl, —O-3-methyl-1-butyl, —O-2-methyl-3-butyl, —O-2,2-dimethyl-1-propyl, —O-2-methyl-1-pentyl, 3-O-methyl-1-pentyl, —O-4-methyl-1-pentyl, —O-2-methyl-2-pentyl, —O-3-methyl-2-pentyl, —O-4-methyl-2-pentyl, —O-2,2-dimethyl-1-butyl, —O-3,3-dimethyl-1-butyl, —O-2-ethyl-1-butyl, —O-butyl, —O-isobutyl, —O-1 butyl, —O-pentyl, —O-isopentyl, —O-neopentyl and —O-hexyl. Longer alkoxy groups include —O-heptyl, —O-octyl, —O-nonyl and —O-decyl groups. The alkoxy groups may also be isotopologues of the natural abundance alkoxy groups by being enriched in isotopes of carbon, oxygen and/or hydrogen (i.e., deuterium or tritium).

As used herein, and unless otherwise specified, the term “cycloalkyl” or “carbocyclyl” means a species of alkyl, which is cyclic and contains from 3 to 15, 3 to 9, 3 to 6, or 3 to 5 carbon atoms, without alternating or resonating double bonds between carbon atoms. It may contain from 1 to 4 rings. Examples of unsubstituted cycloalkyls include, but are not limited to, cyclopropyl, cyclobutyl, cyclopentyl, cyclohexyl, and adamantyl. A cycloalkyl may be substituted with one or more substituents. In some embodiments, a cycloalkyl may be a cycloalkyl fused with aryl or heteroaryl groups.

As used herein, and unless otherwise specified, the term “heterocycloalkyl” or “heterocyclyl” means a cycloalkyl in which one or more, in some embodiments, 1 to 3, carbon atoms are replaced by heteroatoms such as, but not limited to, N, S, and O. In some embodiments, a heterocycloalkyl group contains from 3 to 15, 3 to 9, 3 to 6, or 3 to 5 carbon and hetero atoms. In some embodiments, a heterocycloalkyl may be a heterocycloalkyl fused with aryl or heteroaryl groups. When a prefix such as C₃₋₆ is used to refer to a heterocycloalkyl group, the number of carbons (3-6, in this example) is meant to include the heteroatoms as well. For example, a C₃₋₆ heterocycloalkyl group is meant to include, for example, tetrahydropyranyl (five carbon atoms and one heteroatom replacing a carbon atom).

As used herein, and unless otherwise specified, the term “aryl” means a carbocyclic aromatic ring containing from 5 to 14 ring atoms. The ring atoms of a carbocyclic aryl group are all carbon atoms. Aryl ring structures include compounds having one or more ring structures such as mono-, bi-, or tricyclic compounds as well as benzo-fused carbocyclic moieties such as 5,6,7,8-tetrahydronaphthyl and the like. Specifically, the aryl group may be a mono-, bi-, or tricyclic ring. Representative aryl groups include phenyl, anthracenyl, fluorenyl, indenyl, azulenyl, phenanthrenyl and naphthyl.

As used herein, and unless otherwise specified, the term “heteroaryl” refers to a monocyclic or multicyclic aromatic ring system, in certain embodiments, of about 5 to about 15 members where one or more, in some embodiments, 1 to 3, of the atoms in the ring system is a heteroatom, that is, an element other than carbon, including but not limited to, N, O or S. The heteroaryl group may be optionally fused to a benzene ring. Heteroaryl groups include, but are not limited to, furyl, imidazolyl, indolinyl, pyrrolidinyl, pyrimidinyl, tetrazolyl, thienyl, pyridyl, pyrrolyl, N-methylpyrrolyl, quinolinyl and isoquinolinyl.

As used herein, and unless otherwise indicated, the term “alcohol” means any compound substituted with an —OH group. The alcohol group may also be isotopologues of the natural abundance alcohol groups by being enriched in isotopes of oxygen and/or hydrogen (i.e., deuterium or tritium).

As used herein, and unless otherwise indicated, the term “amino” or “amino group” means a monovalent group of the formula —NH₂, —NH(alkyl), —NH(aryl), —N(alkyl)₂, —N(aryl)₂ or —N(alkyl)(aryl). The amino groups may also be isotopologues of the natural abundance amino groups by being enriched in isotopes of carbon, nitrogen and/or hydrogen (i.e., deuterium or tritium).

Unless otherwise indicated, the compounds provided herein, including intermediates useful for the preparation of the compounds provided herein, which contain reactive functional groups (such as, without limitation, carboxy, hydroxy, and amino moieties) also include protected derivatives thereof. “Protected derivatives” are those compounds in which a reactive site or sites are blocked with one or more protecting groups (also known as blocking groups). Suitable protecting groups for carboxy moieties include benzyl, 1-butyl, and the like as well as isotopologues of the like. Suitable protecting groups for amino and amido groups include acetyl, trifluoroacetyl, 1-butyloxycarbonyl, benzyloxycarbonyl, and the like. Suitable protecting groups for hydroxy include benzyl and the like. Other suitable protecting groups are well known to those of ordinary skill in the art. The choice and use of protecting groups and the reaction conditions to install and remove protecting groups are described in Greene's Protective Groups in Organic Synthesis, 4th edition, John Wiley & Sons, New York, 2007, which is incorporated herein by reference in its entirety.

Amino protecting groups known in the art include those described in detail in T. W. Green, Protective Groups in Organic Synthesis. Amino protecting groups include, but are not limited to, —OH, —OR^{aa}, —N(R^{cc})₂, —C(═O)R^{aa}, —C(═O)N(R^{cc})₂, —CO₂R^{aa}, —SO₂R^{aa}, —C(═NR^{cc})R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, C₁₋₁₀ alkyl (e.g., aralkyl groups), C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl groups, wherein each alkyl, alkenyl, alkynyl, carbocyclyl, heterocyclyl, aralkyl, aryl, and heteroaryl is independently substituted with 0, 1, 2, 3, 4, or 5 R^{dd} groups; wherein
each instance of R^{aa} is, independently, selected from C₁₋₁₀ alkyl, C₁₋₁₀ perhaloalkyl, C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl, wherein each alkyl, alkenyl, alkynyl, carbocyclyl, heterocyclyl, aryl, and heteroaryl is independently substituted with 0, 1, 2, 3, 4, or 5 R^{dd} groups;
each instance of R^{bb} is, independently, selected from hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)₂R^{aa}, —P(═O)(R^{aa})₂, —P(═O)₂N(R^{cc})₂, —P(═O)(NR^{cc})₂, C₁₋₁₀ alkyl, C₁₋₁₀ perhaloalkyl, C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl, or two R^{cc} groups attached to an N atom are joined to form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups.
each instance of R^{cc} is, independently, selected from hydrogen, C₁₋₁₀ alkyl, C₁₋₁₀ perhaloalkyl, C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl, or two R^{cc} groups attached to an N atom are joined to form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups.
each instance of R^{dd} is, independently, selected from halogen, —CN, —NO₂, —N₃, —SO₂H, —SO₃H, —OH, —OR^{ee}, —ON(R^{ff})₂, —N(R^{ff})₂, —N(R^{ff})₃⁺X⁻, —N(OR^{ee})R^{ff}, —SH, —SR^{ee}, —SSR^{ee}, —C(═O)R^{ee}, —CO₂H, —CO₂R^{ee}, —OC(═O)R^{cc}, —OCO₂R^{ee}, —C(═O)N(R^{ff})₂, —OC(═O)N(R^{ff})₂, —NR^{ff}C(═O)R^{ee}, —NR^{ff}CO₂R^{ee}, —NR^{ff}C(═O)N(R^{ff})₂, —C(═NR^{ff})OR^{ee}, —OC(—NR^{ff})R^{ee}, —OC(═NR^{ff})OR^{ee}, —C(═NR^{ff})N(R^{ff})₂, —OC(═NR^{ff})N(R^{ff})₂, —NR^{ff}C(═NR^{ff})N(R^{ff})₂, —NR^{ff}SO₂R^{ee}, —SO₂N(R^{ff})₂, —SO₂R^{ee}, —SO₂OR^{ee}, —OSO₂R^{ee}, —S(═O)R^{ee}, —Si(R^{ee})₃, —OSi(R^{ee})₃, —C(═S)N(R^{ff})₂, —C(═O)SR^{cc}, —C(═S)SR^{ee}, —SC(═S)SR^{ee}, —P(═O)₂R^{ee}, —P(═O)(R^{ee})₂, —OP(═O)(R^{ee})₂, —OP(═O)(OR^{ee})₂, C₁₋₆ alkyl, C₁₋₆ perhaloalkyl, C₂₋₆ alkenyl, C₂₋₆ alkynyl, C₃₋₁₀ carbocyclyl, 3-10 membered heterocyclyl, C₆₋₁₀ aryl, 5-10 membered heteroaryl, wherein each alkyl, alkenyl, alkynyl, carbocyclyl, heterocyclyl, aryl, and heteroaryl is independently substituted with 0, 1, 2, 3, 4, or 5 R^{gg} groups, or two geminal R^{dd} substituents can be joined to form ═O or ═S.
each instance of R^{ee} is, independently, selected from C₁₋₆ alkyl, C₁₋₆ perhaloalkyl, C₂₋₆ alkenyl, C₂₋₆ alkynyl, C₃₋₁₀ carbocyclyl, C₆₋₁₀ aryl, 3-10 membered heterocyclyl, and 3-10 membered heteroaryl, wherein each alkyl, alkenyl, alkynyl, carbocyclyl, heterocyclyl, aryl, and heteroaryl is independently substituted with 0, 1, 2, 3, 4, or 5 R^{gg} groups;
each instance of R^{ff} is, independently, selected from hydrogen, C₁₋₆ alkyl, C₁₋₆ perhaloalkyl, C₂₋₆ alkenyl, C₂₋₆ alkynyl, C₃₋₁₀ carbocyclyl, 3-10 membered heterocyclyl, C₆₋₁₀ aryl and 5-10 membered heteroaryl, or two R^{ff} groups attached to an N atom are joined to form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{gg} groups; and
each instance of R^{gg} is, independently, halogen, —CN, —NO₂, —N₃, —SO₂H, —SO₃H, —OH, OC₁₋₆ alkyl, —ON(C₁₋₆ alkyl)₂, —N(C₁₋₆ alkyl)₂, —N(C₁₋₆ alkyl)₃X, —NH(C₁₋₆ alkyl)₂X, —NH₂(C₁₋₆ alkyl)X, —NH₃X, —N(OC₁₋₆ alkyl)(C₁₋₆ alkyl), —N(OH)(C₁₋₆ alkyl), —NH(OH), —SH, —SC₁₋₆ alkyl, —SS(C₁₋₆ alkyl), —C(═O)(C₁₋₆ alkyl), —CO₂H, —CO₂(C₁₋₆ alkyl), —OC(═O)(C₁₋₆ alkyl), —OCO₂(C₁₋₆ alkyl), —C(═O)NH₂, —C(═O)N(C₁₋₆ alkyl)₂, —OC(═O)NH(C₁₋₆ alkyl), —NHC(═O)(C₁₋₆ alkyl), —N(C₁₋₆ alkyl)C(═O)(C₁₋₆ alkyl), —NHCO₂(C₁₋₆ alkyl), —NHC(═O)N(C₁₋₆ alkyl)₂, —NHC(═O)NH(C₁₋₆ alkyl), —NHC(═O)NH₂, —C(═NH)O(C₁₋₆ alkyl), —OC(═NH)(C₁₋₆ alkyl), —OC(═NH)OC₁₋₆ alkyl, —C(═NH)N(C₁₋₆ alkyl)₂, —C(═NH)NH(C₁₋₆ alkyl), —C(═NH)NH₂, —OC(═NH)N(C₁₋₆ alkyl)₂, —OC(NH)NH(C₁₋₆ alkyl), —OC(NH)NH₂, —NHC(NH)N(C₁₋₆ alkyl)₂, —NHC(═NH)NH₂, —NHSO₂(C₁₋₆ alkyl), —SO₂N(C₁₋₆ alkyl)₂, —SO₂NH(C₁₋₆ alkyl), —SO₂NH₂, —SO₂C₁₋₆ alkyl, —SO₂OC₁₋₆ alkyl, —OSO₂C₁₋₆ alkyl, —SOC₁₋₆ alkyl, —Si(C₁₋₆ alkyl)₃, —OSi(C₁₋₆ alkyl)₃ —C(═S)N(C₁₋₆ alkyl)₂, C(═S)NH(C₁₋₆ alkyl), C(═S)NH₂, —C(═O)S(C₁₋₆ alkyl), —C(═S)SC₁₋₆ alkyl, —SC(═S)SC₁₋₆ alkyl, —P(═O)₂(C₁₋₆ alkyl), —P(═O)(C₁₋₆ alkyl)₂, —OP(═O)(C₁₋₆ alkyl)₂, —OP(═O)(OC₁₋₆ alkyl)₂, C₁₋₆ alkyl, C₁₋₆ perhaloalkyl, C₂₋₆ alkenyl, C₂₋₆ alkynyl, C₃₋₁₀ carbocyclyl, C₆₋₁₀ aryl, 3-10 membered heterocyclyl, 5-10 membered heteroaryl; or two geminal R^{gg} substituents can be joined to form ═O or ═S;
wherein X⁻ is a counterion.

As used herein, a “counterion” is a negatively charged group associated with a positively charged quarternary amine in order to maintain electronic neutrality. Exemplary counterions include halide ions (e.g., F⁻, Cl⁻, Br⁻, I⁻), NO₃⁻, ClO₄⁻, OH⁻, H₂PO₄⁻, HSO₄⁻, sulfonate ions (e.g., methansulfonate, trifluoromethanesulfonate, p-toluenesulfonate, benzenesulfonate, 10-camphor sulfonate, naphthalene-2-sulfonate, naphthalene-1-sulfonic acid-5-sulfonate, ethan-1-sulfonic acid-2-sulfonate, and the like) and carboxylate ions (e.g., acetate, ethanoate, propanoate, benzoate, glycerate, lactate, tartrate, glycolate, and the like). Counterions also include chrial counterions, some of which may be useful for chiral resolution of racemic mixtures. Exemplary chiral counterions include (S)-(+) mandelic acid, (D)-(+) tartaric acid, (+) 2,3-dibenzoyl-D-tartaric acid, N-Acetyl-L-leucine, and N-Acetyl-L-phenylalanine.

For example, amino protecting groups such as amide groups (e.g., —C(═O)R^{aa}) include, but are not limited to, formamide, acetamide, chloroacetamide, trichloroacetamide, trifluoroacetamide, phenylacetamide, 3-phenylpropanamide, picolinamide, 3-pyridylcarboxamide, N-benzoylphenylalanyl derivative, benzamide, p-phenylbenzamide, o-nitophenylacetamide, o-nitrophenoxyacetamide, acetoacetamide, (N′-dithiobenzyloxycarbonylamino)acetamide, 3-(p-hydroxyphenyl)propanamide, 3-(0-nitrophenyl)propanamide, 2-methyl-2-(o-nitrophenoxy)propanamide, 2-methyl-2-(0-phenylazophenoxy)propanamide, 4-chlorobutanamide, 3-methyl-3-nitrobutanamide, o-nitrocinnamide, N-acetylmethionine derivative, o-nitrobenzamide and o-(benzoyloxymethyl)benzamide.

Amino protecting groups such as carbamate groups (e.g., —C(═O)OR^{aa}) include, but are not limited to, methyl carbamate, ethyl carbamante, 9-fluorenylmethyl carbamate (Fmoc), 9-(2-sulfo)fluorenylmethyl carbamate, 9-(2,7-dibromo)fluoroenylmethyl carbamate, 2,7-di-1-butyl-[9-(10,10-dioxo-10, 10, 10, 10-tetrahydrothioxanthyl)]methyl carbamate (DBD Tmoc), 4-methoxyphenacyl carbamate (Phenoc), 2,2,2-trichloroethyl carbamate (Troc), 2-trimethylsilylethyl carbamate (Teoc), 2-phenylethyl carbamate (hZ), 1-(1-adamantyl)-1-methylethyl carbamate (Adpoc), 1,1-dimethyl-2-haloethyl carbamate, 1,1-dimethyl-2,2-dibromoethyl carbamate (DB-1-BOC), 1,1-dimethyl-2,2,2-trichloroethyl carbamate (TCBOC), 1-methyl-1-(4-biphenylyl)ethyl carbamate (Bpoc), 1-(3,5-di-1-butylphenyl)-1-methylethyl carbamate (t-Bumeoc), 2-(2′- and 4′-pyridyl)ethyl carbamate (Pyoc), 2-(N,N-dicyclohexylcarboxamido)ethyl carbamate, 1-butyl carbamate (Boc), 1-adamantyl carbamate (Adoc), vinyl carbamate (Voc), allyl carbamate (Alloc), 1-isopropylallyl carbamate (Ipaoc), cinnamyl carbamate (Coc), 4-nitrocinnamyl carbamate (Noc), 8-quinolyl carbamate, N-hydroxypiperidinyl carbamate, alkyldithio carbamate, benzyl carbamate (Cbz), p-methoxybenzyl carbamate (Moz), p-nitobenzyl carbamate, p-bromobenzyl carbamate, p-chlorobenzyl carbamate, 2,4-dichlorobenzyl carbamate, 4-methylsulfinylbenzyl carbamate (Msz), 9-anthrylmethyl carbamate, diphenylmethyl carbamate, 2-methylthioethyl carbamate, 2-methylsulfonylethyl carbamate, 2-(p-toluenesulfonyl)ethyl carbamate, [2-(1,3-dithianyl)]methyl carbamate (Dmoc), 4-methylthiophenyl carbamate (Mtpc), 2,4-dimethylthiophenyl carbamate (Bmpc), 2-phosphonioethyl carbamate (Peoc), 2-triphenylphosphonioisopropyl carbamate (Ppoc), 1,1-dimethyl-2-cyanoethyl carbamate, m-chloro-p-acyloxybenzyl carbamate, p-(dihydroxyboryl)benzyl carbamate, 5-benzisoxazolylmethyl carbamate, 2-(trifluoromethyl)-6-chromonylmethyl carbamate (Tcroc), m-nitrophenyl carbamate, 3,5-dimethoxybenzyl carbamate, o-nitrobenzyl carbamate, 3,4-dimethoxy-6-nitrobenzyl carbamate, phenyl(o-nitrophenyl)methyl carbamate, 1-amyl carbamate, S-benzyl thiocarbamate, p cyanobenzyl carbamate, cyclobutyl carbamate, cyclohexyl carbamate, cyclopentyl carbamate, cyclopropylmethyl carbamate, p-decyloxybenzyl carbamate, 2,2-dimethoxycarbonylvinyl carbamate, o-(N,N-dimethylcarboxamido)benzyl carbamate, 1,1-dimethyl-3-(N,N-dimethylcarboxamido)propyl carbamate, 1,1-dimethylpropynyl carbamate, di(2-pyridyl)methyl carbamate, 2-furanylmethyl carbamate, 2-iodoethyl carbamate, isoborynl carbamate, isobutyl carbamate, isonicotinyl carbamate, p-(p′-methoxyphenylazo)benzyl carbamate, 1-methylcyclobutyl carbamate, 1-methylcyclohexyl carbamate, 1-methyl-1-cyclopropylmethyl carbamate, 1-methyl-1-(3,5-dimethoxyphenyl)ethyl carbamate, 1-methyl-1-(p-phenylazophenyl)ethyl carbamate, 1-methyl-1-phenylethyl carbamate, 1-methyl-1-(4-pyridyl)ethyl carbamate, phenyl carbamate, p-(phenylazo)benzyl carbamate, 2,4,6-tri-1-butylphenyl carbamate, 4-(trimethylammonium)benzyl carbamate, and 2,4,6-trimethylbenzyl carbamate.

Amino protecting groups such as sulfonamide groups (e.g., —S(═O)₂R^{aa}) include, but are not limited to, p-toluenesulfonamide (Ts), benzenesulfonamide, 2,3,6,-trimethyl-4-methoxybenzenesulfonamide (Mtr), 2,4,6-trimethoxybenzenesulfonamide (Mtb), 2,6-dimethyl-4-methoxybenzenesulfonamide (Pme), 2,3,5,6-tetramethyl-4-methoxybenzenesulfonamide (Mte), 4-methoxybenzenesulfonamide (Mbs), 2,4,6-trimethylbenzenesulfonamide (Mts), 2,6-dimethoxy-4-methylbenzenesulfonamide (iMds), 2,2,5,7,8-pentamethylchroman-6-sulfonamide (Pmc), methanesulfonamide (Ms), β-trimethylsilylethanesulfonamide (SES), 9-anthracenesulfonamide, 4-(4′,8′-dimethoxynaphthylmethyl)benzenesulfonamide (DNMBS), benzylsulfonamide, trifluoromethylsulfonamide, and phenacylsulfonamide.

Other amino protecting groups include, but are not limited to, phenothiazinyl-(10)-carbonyl derivative, N′-p-toluenesulfonylaminocarbonyl derivative, N′-phenylaminothiocarbonyl derivative, N-benzoylphenylalanyl derivative, N-acetylmethionine derivative, 4,5-diphenyl-3-oxazolin-2-one, N-phthalimide, N-dithiasuccinimide (Dts), N-2,3-diphenylmaleimide, N-2,5-dimethylpyrrole, N-1,1,4,4-tetramethyldisilylazacyclopentane adduct (STABASE), 5-substituted 1,3-dimethyl-1,3,5-triazacyclohexan-2-one, 5-substituted 1,3-dibenzyl-1,3,5-triazacyclohexan-2-one, 1-substituted 3,5-dinitro-4-pyridone, N-methylamine, N-allylamine, N-[2-(trimethylsilyl)ethoxy]methylamine (SEM), N-3-acetoxypropylamine, N (1-isopropyl-4-nitro-2-oxo-3-pyroolin-3-yl)amine, quaternary ammonium salts, N-benzylamine, N-di(4-methoxyphenyl)methylamine, N-5-dibenzosuberylamine, N-triphenylmethylamine (Tr), N-[(4-methoxyphenyl)diphenylmethyl]amine (MMTr), N-9-phenylfluorenylamine (PhF), N-2,7-dichloro-9-fluorenylmethyleneamine, N-ferrocenylmethylamino (Fcm), N-2-picolylamino N′-oxide, N-1,1-dimethylthiomethyleneamine, N-benzylideneamine, N p-methoxybenzylideneamine, N-diphenylmethyleneamine, N-[(2-pyridyl)mesityl]methyleneamine, N—(N′,N′ dimethylaminomethylene)amine, N,N′-isopropylidenediamine, N p-nitrobenzylideneamine, N-salicylideneamine, N-5-chlorosalicylideneamine, N-(5-chloro-2-hydroxyphenyl)phenylmethyleneamine, N-cyclohexylideneamine, N-(5,5-dimethyl-3-oxo-1-cyclohexenyl)amine, N-borane derivative, N-diphenylborinic acid derivative, N-[phenyl(pentacarbonylchromium- or tungsten)carbonyl]amine, N-copper chelate, N-zinc chelate, N-nitroamine, N-nitrosoamine, amine N oxide, diphenylphosphinamide (Dpp), dimethylthiophosphinamide (Mpt), diphenylthiophosphinamide (Ppt), dialkyl phosphoramidates, dibenzyl phosphoramidate, diphenyl phosphoramidate, benzenesulfenamide, o-nitrobenzenesulfenamide (Nps), 2,4-dinitrobenzenesulfenamide, pentachlorobenzenesulfenamide, 2-nitro-4-methoxybenzenesulfenamide, triphenylmethylsulfenamide and 3-nitropyridinesulfenamide (Npys).

As used herein, and unless otherwise indicated, the term “hydroxyl protecting group” refers to a protecting group suitable for preventing undesired reactions at a hydroxyl group. Examples of hydroxyl protecting groups include, but are not limited to, allyl, methyl, 2-methoxyethoxymethyl (MEM), methoxymethyl (MOM), methoxythiomethyl, 1-butoxymethyl, tri-isopropylsilyloxymethyl (TOM), ethyl, 1-ethoxyehtyl, isopropyl, 1-butyl, benzyl, trityl (Tr), dimethoxytrityl (DMT), monomethoxytrityl (MMT), p-methoxybenzyl (PMB), acetyl, chloroacetyl, trichloroacetyl, trifluoroacetyl, pivaloyl (Piv), benzoyl, p-phenylbenzoyl, trimethylsilyl (TMS), triisopropylsilyl (TIPS), 1-butyldimethylsilyl (TBDMS), and tetrahydropyranyl. Additional examples of hydroxyl protecting groups are described in Greene's Protective Groups in Organic Synthesis, 4th edition, John Wiley & Sons, New York, 2007, which is incorporated herein by reference in its entirety.

As used herein, and unless otherwise indicated, acronyms or symbols for groups or reagents have the following definition: HPLC=high performance liquid chromatography; THF=tetrahydrofuran; CH₃CN=acetonitrile; HOAc=acetic acid; DCM=dichloromethane; IPA=isopropyl alcohol; MTBE=methyl tert-butyl ether, CPME=cyclopentyl methyl ether; DMF=dimethylformamide; NMP=N-methyl-2-pyrrolidone; EtOAc=ethyl acetate; MsCI=mesyl chloride; DIEA=diisopropylethylamine; TEA=triethylamine.

As used herein, and unless otherwise indicated, the term “substituted” or “substitution,” when used to describe a chemical structure or moiety, refers to a derivative of that structure or moiety wherein one or more of its hydrogen atoms is replaced with a substituent such as, but not limited to: alkyl, alkenyl, alkynyl, and cycloalkyl; alkoxyalkyl; aroyl; halo; haloalkyl (e.g., trifluoromethyl); heterocycloalkyl; haloalkoxy (e.g., trifluoromethoxy); hydroxy; alkoxy; cycloalkyloxy; heterocylooxy; oxo; alkanoyl; aryl; heteroaryl (e.g., indolyl, imidazolyl, furyl, thienyl, thiazolyl, pyrrolidyl, pyridyl, and pyrimidyl); arylalkyl; alkylaryl; heteroaryl; heteroarylalkyl; alkylheteroaryl; heterocyclo; heterocycloalkyl-alkyl; aryloxy, alkanoyloxy; amino; alkylamino; arylamino; arylalkylamino; cycloalkylamino; heterocycloamino; mono- and di-substituted amino; alkanoylamino; aroylamino; aralkanoylamino; aminoalkyl; carbamyl (e.g., CONH₂); substituted carbamyl (e.g., CONH-alkyl, CONH-aryl, CONH-arylalkyl or instances where there are two substituents on the nitrogen); carbonyl; alkoxycarbonyl; carboxy; cyano; ester; ether; guanidino; nitro; sulfonyl; alkylsulfonyl; arylsulfonyl; arylalkylsulfonyl; sulfonamido (e.g., SO₂NH₂); substituted sulfonamido; thiol; alkylthio; arylthio; arylalkylthio; cycloalkylthio; heterocyclothio; alkylthiono; arylthiono; and arylalkylthiono. In some embodiments, a substituent itself may be substituted with one or more chemical moieties such as, but not limited to, those described herein.

As used herein, and unless otherwise indicated, the terms “about” and “approximately” are used to specify that the values given are approximate. For example, the term “about,” where it is used in connection with reaction temperatures, denotes that the temperature deviations within 30%, 25%, 20%, 15%, 10%, or 5% are encompassed by the temperature indicated. Similarly, the term “about,” where it is used in connection with reaction time, denotes that the time period deviations within 30%, 25%, 20%, 15%, 10%, or 5% are encompassed by the time period indicated.

As used herein, and unless otherwise specified, the terms “about” and “approximately,” when used in connection with a numeric value or a range of values which is provided to characterize a particular solid form, e.g., a specific temperature or temperature range, such as, for example, that describing a melting, dehydration, desolvation or glass transition temperature; a mass change, such as, for example, a mass change as a function of temperature or humidity; a solvent or water content, in terms of, for example, mass or a percentage; or a peak position, such as, for example, in analysis by IR or Raman spectroscopy or XRPD; indicate that the value or range of values may deviate to an extent deemed reasonable to one of ordinary skill in the art while still describing the particular solid form. For example, in particular embodiments, the terms “about” and “approximately,” when used in this context, indicate that the numeric value or range of values may vary within 25%, 20%, 15%, 10%, 9%, 8%, 7%, 6%, 5%, 4%, 3%, 2%, 1.5%, 1%, 0.5%, or 0.25% of the recited value or range of values. For example, in some embodiment, the value of XRPD peak position may vary by up to +0.2 degrees 2θ while still describing the particular XRPD peak. In one embodiment, the value of XRPD peak position may vary by up to +0.1 degrees 2θ. As used herein, a tilde (i.e., “˜”) preceding a numerical value or range of values indicates “about” or “approximately.”

As used herein, and unless otherwise indicated, the term “hydrogenation” refers to a chemical process that adds hydrogen atom to an unsaturated bond.

As used herein, and unless otherwise indicated, an “isotopologue” is an isotopically enriched compound. The term “isotopically enriched” refers to an atom having an isotopic composition other than the natural isotopic composition of that atom. “Isotopically enriched” may also refer to a compound containing at least one atom having an isotopic composition other than the natural isotopic composition of that atom. The term “isotopic composition” refers to the amount of each isotope present for a given atom, and “natural isotopic composition” refers to the naturally occurring isotopic composition or abundance for a given atom.

The disclosure can be understood more fully by reference to the following detailed description and illustrative examples, which are intended to exemplify non-limiting embodiments.

Although most embodiments and examples provided herein are directed to the (S)-enantiomer of a compound, it is to be understood that the corresponding (R)-enantiomer of a compound can be prepared by the provided processes when the stereochemistry of chiral reactant, reagent, solvent, catalyst, ligand or the like is reversed.

### 6.2 Processes

In some embodiments, provided herein are processes for preparing a compound of Formula (I):

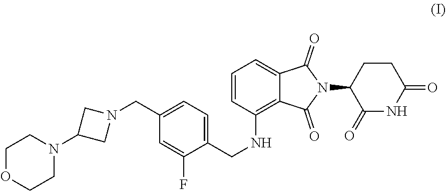 [SMILES: O=C1CC[C@H](N2C(=O)c3cccc(NCc4ccc(CN5CC(N6CCOCC6)C5)cc4F)c3C2=O)C(=O)N1]

or a salt, solvate, hydrate, enantiomer, mixture of enantiomers, or isotopologue thereof, comprising:
(step 1.0) cyclizing a compound of Formula (II):

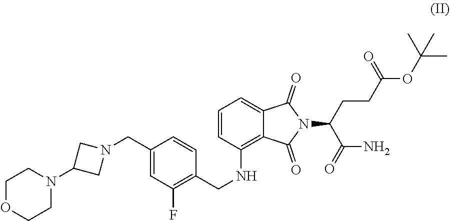 [SMILES: CC(C)(C)OC(=O)CC[C@@H](C(N)=O)N1C(=O)c2cccc(NCc3ccc(CN4CC(N5CCOCC5)C4)cc3F)c2C1=O]

or a salt, solvate, hydrate, enantiomer, mixture of enantiomers, or isotopologue thereof, to provide a compound of Formula (I), or a salt, solvate, hydrate, enantiomer, mixture of enantiomers, or isotopologue thereof; and
(step 1.1) optionally converting the compound of Formula (I), or a salt, solvate, hydrate, enantiomer, mixture of enantiomers, or isotopologue thereof, to a salt of the compound.

In some embodiments, step 1.0 occurs in the presence of an acid. In some embodiments, step 1.0 occurs in the presence of an inorganic acid. In some embodiments, step 1.0 occurs in the presence of hydrochloric acid, sulfuric acid, nitric acid, or phosphoric acid. In one embodiment, step 1.0 occurs in the presence of hydrochloric acid.

In some embodiments, step 1.0 occurs in the presence of an organic acid. In some embodiments, step 1.0 occurs in the presence of R^{b}COOH wherein R^{b} is hydrogen, substituted or unsubstituted C₁₋₁₀ alkyl, substituted or unsubstituted C₁₋₁₀ haloalkyl, or substituted or unsubstituted C₅₋₁₄ aryl. In some embodiments, step 1.0 occurs in the presence of formic acid, acetic acid, trifluoroacetic acid, or benzoic acid.

In some embodiments, step 1.0 occurs in the presence of R^{b}SO₃H wherein R^{b} is hydrogen, substituted or unsubstituted C₁₋₁₀ alkyl, substituted or unsubstituted C₁₋₁₀ haloalkyl, or substituted or unsubstituted C₅₋₁₄ aryl. In some embodiments, step 1.0 occurs in the presence of sulfonic acid, benzenesulfonic acid, p-toluenesulfonic acid, camphorsulfonic acid, methanesulfonic acid, or trifluoromethanesulfonic acid. In one embodiment, step 1.0 occurs in the presence of benzenesulfonic acid.

In some embodiments, the compound of Formula (I), or a salt, solvate, hydrate, enantiomer, mixture of enantiomers, or isotopologue thereof, prepared in step 1.0 is a salt of the compound of Formula (I). In some embodiments, the salt of the compound of Formula (I) may result from protonation of one or more of its nitrogen atoms. In some embodiments, the salt of the compound of Formula (I) may be a chloride, bromide, iodide, sulfate, nitrate, phosphate, acetate, formate, trifluoroacetate, benzoate, sulfonate, besylate, tosylate, camphorsulfonate, mesylate or triflate salt of the compound of Formula (I). In one embodiment, a besylate salt of the compound of Formula (I) is prepared in step 1.0. In one embodiment, the besylate salt is a bis-besylate salt.

In some embodiments, the molar ratio of the compound of Formula (II) to the acid is from about 1:4 to about 1:7. In one embodiment, the molar ratio of the compound of Formula (II) to the acid is about 1:5.5.

Step 1.0 may occur in a solvent suitable for the cyclization reaction. In some embodiments, the solvent is diethyl ether, methyl tert-butyl ether, cyclopentyl methyl ether, 1,4-dioxane, tetrahydrofuran, methyltetrahydrofuran, ethyl acetate, isopropyl acetate, acetonitrile, methanol, ethanol, isopropyl alcohol, water, dichloromethane, dimethylformamide, dimethyl sulfoxide, glyme, diglyme, dimethylacetamide, or N-methyl-2-pyrrolidone, or a mixture thereof. In one embodiment, the solvent is acetonitrile. In another embodiments, the solvent is a mixture of acetonitrile and methyl tert-butyl ether. In yet another embodiment, the solvent is a mixture of acetonitrile and isopropyl acetate. In still another embodiment, the solvent is a mixture of acetonitrile, methyltetrahydrofuran and, optionally, water.

In some embodiments, only a stoichiometric amount of water is added. In some embodiments, the molar ratio of the compound of Formula (II) to water is from about 1:1 to about 1:3. In one embodiment, the molar ratio of the compound of Formula (II) to water is 1:2.

Step 1.0 may occur at a reaction temperature suitable for the cyclization reaction. In some embodiments, the reaction temperature is from about 20° C. to about 100° C. In some embodiments, step 1.0 occurs at the refluxing temperature of the solvent. In one embodiment, the reaction temperature is about 55° C.

In some embodiments, the reaction time for step 1.0 is from about 10 hour to about 20 hours. In one embodiment, the reaction time is about 16 hours.

In one embodiment, step 1.0 occurs in the presence of benzenesulfonic acid, the solvent is a mixture of acetonitrile and methyltetrahydrofuran, and a bis-besylate salt of the compound of Formula (I) is prepared.

In some embodiments, in step 1.1 the compound of Formula (I), or a salt, solvate, hydrate, enantiomer, mixture of enantiomers, or isotopologue thereof is converted to a different salt of the compound. In one embodiment, the compound of Formula (I), or a salt, solvate, hydrate, enantiomer, mixture of enantiomers, or isotopologue thereof is converted to a hydrochloride salt of the compound.

In some embodiments, in step 1.1 a salt of the compound of Formula (I) is contacted with a basic aqueous solution which is subsequently acidified. In some embodiments, basic aqueous solution consists of a bicarbonate solution. In some embodiments, acidification comprises addition of hydrochloric acid, or a solution thereof.

In some embodiments, step 1.1 occurs in a biphasic mixture comprising an aqueous solution and an organic solvent. In some embodiments, the organic solvent is diethyl ether, methyl tert-butyl ether, cyclopentyl methyl ether, 1,4-dioxane, tetrahydrofuran, methyltetrahydrofuran, ethyl acetate, isopropyl acetate, acetonitrile, methanol, ethanol, isopropyl alcohol, dichloromethane, dimethylformamide, dimethyl sulfoxide, glyme, diglyme, dimethylacetamide, or N-methyl-2-pyrrolidone, or a mixture thereof. In one embodiment, the organic solvent is methyltetrahydrofuran. In another embodiment, the organic solvent is a mixture of ethyl acetate or isopropyl alcohol.

In some embodiments, step 1.1 occurs at a reaction temperature of from about 0° C. to about 25° C. In one embodiment, the reaction temperature is about 15° C.

In one embodiment of step 1.1, a bis-besylate salt of the compound of Formula (I) is converted to a hydrochloride salt of the compound of Formula (I). In one embodiment, the bis-besylate salt (e.g., in a solvent of a mixture of ethyl acetate or isopropyl alcohol) is neutralized or basified by addition of aqueous potassium bicarbonate solution, and then acidified by addition of hydrochloric acid to provide the hydrochloride salt. In one embodiment, the hydrochloride salt is subject to further wet-milling and/or co-milling.

In some embodiments, provided herein are processes for preparing a compound of Formula (II), or a salt, solvate, hydrate, enantiomer, mixture of enantiomers, or isotopologue thereof, comprising:
(step 2.a) reacting a compound of Formula (II-A):

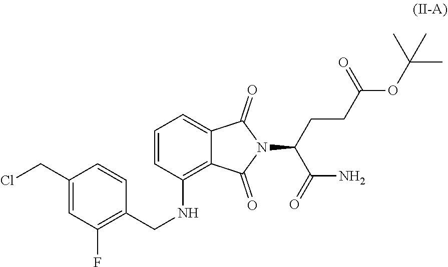 [SMILES: CC(C)(C)OC(=O)CC[C@@H](C(N)=O)N1C(=O)c2cccc(NCc3ccc(CCl)cc3F)c2C1=O]

or a salt, solvate, hydrate, enantiomer, mixture of enantiomers, or isotopologue thereof, with 4-(azetidin-3-yl)morpholine, or a salt thereof.

In some embodiments, a salt of 4-(azetidin-3-yl)morpholine is used as one of the starting materials in step 2.a. In one embodiment, a hydrochloride salt of 4-(azetidin-3-yl)morpholine is used.

In some embodiments, the molar ratio of the compound of Formula (II-A) to 4-(azetidin-3-yl)morpholine, or a salt thereof, is from about 2:1 to about 1:2. In one embodiment, the molar ratio of the compound of Formula (II-A) to 4-(azetidin-3-yl)morpholine, or a salt thereof, is about 1:1.

In some embodiments, step 2.a occurs in the presence of base. In some embodiments, step 2.a occurs in the presence of a nitrogen containing base. In some embodiments, step 2.a occurs in the presence of NH₄OH, triethylamine, diisopropylethylamine (DIEA), pyridine, lutidine, 4-dimethylaminopyridine, imidazole, or 1,8-diazabicyclo[5.4.0]undec-7-ene (DBU). In one embodiment, the base is diisopropylethylamine (DIEA).

In some embodiments, the molar ratio of the compound of Formula (II-A) to the base is from about 1:2 to about 1:4. In one embodiment, the molar ratio of the compound of Formula (II-A) to the base is about 1:3.

Step 2.a may occur in a solvent suitable for the reaction. In one embodiment, the solvent is dimethyl sulfoxide.

In some embodiments, step 2.a occurs at a reaction temperature of from about 0° C. to about 40° C. In one embodiment, the reaction temperature is about 30° C.

In some embodiments, step 2.a occurs at a reaction time from from about 8 hours to about 24 hours. In one embodiment, the reaction time is about 16 hours.

In one embodiment, the compound of Formula (II-A) is reacted with a hydrochloride salt of 4-(azetidin-3-yl)morpholine in the presence of diisopropylethylamine as a base, the molar ratio of the compound of Formula (II-A) to 4-(azetidin-3-yl)morpholine is about 1:1, the molar ratio of the compound of Formula (II-A) to base is about 1:3, the solvent is dimethyl sulfoxide. In one embodiment, the reaction temperature is about 30° C., and the reaction time is about 16 hours. In one embodiment, the compound of Formula (II) is purified by selective extraction in ethyl acetate followed by chromatographic separation using silica gel.

In some embodiments, provided herein are processes of for the preparation of a compound of Formula (II-A), or a salt, solvate, hydrate, enantiomer, mixture of enantiomers, or isotopologue thereof, comprising:
(step 2.b) chlorinating a compound of Formula (II-B):

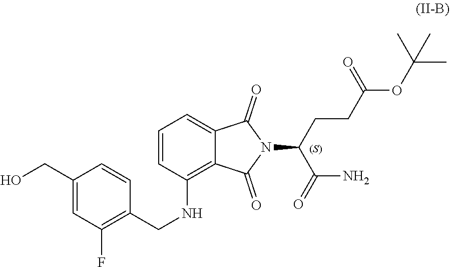

or a salt, solvate, hydrate, enantiomer, mixture of enantiomers, or isotopologue thereof.

Step 2.b may occur in the presence of any chlorinating reagent suitable for the chlorination. In some embodiments, the chlorinating reagent is thionyl chloride, oxalyl chloride, phosphorus trichloride, or mesyl chloride (MsCI). In one embodiment, the chlorinating reagent is mesyl chloride (MsCI).

In some embodiments, the molar ratio of the compound of Formula (II-B) to the chlorinating reagent is from about 1:1 to about 1:3. In one embodiment, the molar ratio of the compound of Formula (II-B) to the chlorinating reagent is about 1:2.

In some embodiments, step 2.b. occurs in the presence of a base. In some embodiments, step 2.b occurs in the presence of a nitrogen containing base. In some embodiments, the base is NH₄OH, triethylamine, diisopropylethylamine (DIEA), pyridine, 4-dimethylaminopyridine, imidazole, or 1,8-diazabicyclo[5.4.0]undec-7-ene (DBU). In one embodiment, the base is diisopropylethylamine (DIEA).

In some embodiments, the molar ratio of the compound of Formula (II-B) to base is from about 1:2 to about 1:4. In one embodiment, the molar ratio of the compound of Formula (II-B) to base is about 1:3.

Step 2.b may occur in a solvent suitable for the reaction. In one embodiment, the solvent is N-methyl-2-pyrrolidone.

In some embodiments, step 2.b occurs at a reaction temperature of from about −5° C. to about 40° C. In one embodiment, the reaction temperature is about 30° C.

In some embodiments, step 2.b occurs at a reaction time of from about 6 hours to about 24 hours. In one embodiment, the reaction time is about 12 hours.

In one embodiment, the compound of Formula (II-B) is reacted with mesyl chloride in the presence of diisopropylethylamine as a base, the molar ratio of the compound of Formula (II-B) to mesyl chloride is about 1:2, the molar ratio of the compound of Formula (II-B) to base is about 1:3, and the solvent is N-methyl-2-pyrrolidone. In one embodiment, the reaction temperature is about 30° C., and the reaction time is about 12 hours. In one embodiment, the compound of Formula (II-A) is purified by selective extraction in methyl tert-butyl ether followed by chromatographic separation using silica gel.

In some embodiments, provided herein are processes for the preparation of a compound of Formula (II-B), or a salt, solvate, hydrate, enantiomer, mixture of enantiomers, or isotopologue thereof, comprising:
(step 2.c) reacting a compound of Formula (V):

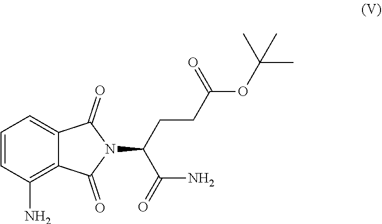 [SMILES: CC(C)(C)OC(=O)CC[C@@H](C(N)=O)N1C(=O)c2cccc(N)c2C1=O]

or a salt, solvate, hydrate, enantiomer, mixture of enantiomers, or isotopologue thereof, with 2-fluoro-4-(hydroxymethyl)benzaldehyde.

In some embodiments, the molar ratio of the compound of Formula (V) to 2-fluoro-4-(hydroxymethyl)benzaldehyde is from about 1:1 to about 1:2. In one embodiment, the molar ratio of the compound of Formula (V) to 2-fluoro-4-(hydroxymethyl)benzaldehyde is about 1:1.3.

In some embodiments, step 2.c occurs in the presence of a reducing agent. In some embodiments, the reducing agent is a borohydride reagent. In some embodiments, the borohydride reagent is sodium borohydride, sodium tri(acetoxy)borohydride or sodium cyanoborohydride. In one embodiment, the borohydride reagent is sodium cyanoborohydride.

In some embodiments, the molar ratio of the compound of Formula (V) to reducing agent is from about 1:1 to about 1:3. In one embodiment, the molar ratio of the compound of Formula (V) to reducing agent is about 1:1.5.

In some embodiments, step 2.c occurs in the presence of a catalyst. In some embodiments, step 2.c occurs in the presence of an acid catalyst. In some embodiments, step 2.c occurs in the presence of a Lewis acid catalyst. In some embodiments, the Lewis acid catalyst is titanium tetra(isopropoxide) or zinc dichloride. In other embodiments, step 2.c occurs in the presence of a Bronsted acid catalyst. In some embodiments, the Bronsted acid catalyst is an organic acid. In some embodiments, the organic acid is a carboxylic acid of the form R^{b}COOH wherein R^{b} is hydrogen, substituted or unsubstituted C₁₋₁₀ alkyl, substituted or unsubstituted C₁₋₁₀ haloalkyl, or substituted or unsubstituted C₅₋₁₄ aryl. In some embodiments, the Bronsted acid catalyst is formic acid, acetic acid, trifluoroacetic acid, or benzoic acid. In one embodiment, step 2.c occurs in the presence of trifluoroacetic acid.

In some embodiments, the molar ratio of the compound of Formula (V) to the catalyst is from about 1:4 to about 1:6. In one embodiment, the molar ratio of the compound of Formula (V) to the catalyst is about 1:5.

Step 2.c may occur in a solvent suitable for the reaction. In one embodiment, the solvent is dichloromethane.

In some embodiments, step 2.c occurs at a reaction temperature of from about −5° C. to about 40° C. In one embodiment, the reaction temperature is about 30° C.

In some embodiments, step 2.c occurs at a reaction time of from about 0.5 hour to about 5 hours. In one embodiment, the reaction time is about 2.5 hours.

In one embodiment, the compound of Formula (V) is reacted with 2-fluoro-4-(hydroxymethyl)benzaldehyde and sodium cyanoborohydride in the presence of trifluoroacetic acid as a catalyst, the molar ratio of the compound of Formula (V) to 2-fluoro-4-(hydroxymethyl)benzaldehyde is about 1:1.3, the molar ratio of the compound of Formula (V) to sodium cyanoborohydride is about 1:1.5, the molar ratio of the compound of Formula (V) to trifluoroacetic acid is about 1:5, and the solvent is dichloromethane. In one embodiment, the reaction temperature is about 30° C., and the reaction time is about 2.5 hours. In one embodiment, the compound of Formula (II-B) is purified by quenching with methanol followed by chromatographic separation using silica gel.

In some embodiments, provided herein are processes for the preparation of a compound of Formula (II), or a salt, solvate, hydrate, enantiomer, mixture of enantiomers, or isotopologue thereof, comprising:
(step 2.0) reacting a compound of Formula (III):

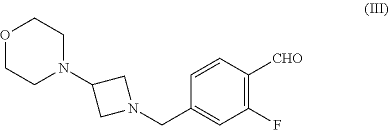 [SMILES: O=Cc1ccc(CN2CC(N3CCOCC3)C2)cc1F]

or a salt, solvate, hydrate, or isotopologue thereof, with a compound of Formula (V):

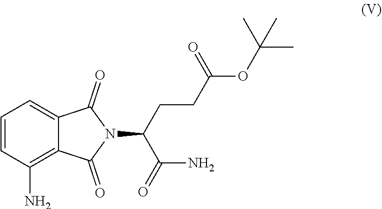 [SMILES: CC(C)(C)OC(=O)CC[C@@H](C(N)=O)N1C(=O)c2cccc(N)c2C1=O]

or a salt, solvate, hydrate, enantiomer, mixture of enantiomers, or isotopologue thereof.

In some embodiments, a salt of the compound of Formula (III) is used in step 2.0. In one embodiment, the salt is a hydrochloride salt. In one embodiment, the salt is an oxalic acid salt. In one embodiment, the salt is a bis-oxalic acid salt. In one embodiment, the salt is a bis-hydrochloride salt.

In some embodiments, the molar ratio of the compound of Formula (V) to the compound of Formula (III) is from about 1:1 to about 1:2. In one embodiment, the molar ratio of the compound of Formula (V) to the compound of Formula (III) is about 1:1.2.

In some embodiments, step 2.0 occurs in the presence of a reducing agent. In some embodiments, the reducing agent is a borohydride reagent. In some embodiments, the borohydride reagent is sodium borohydride, sodium tri(acetoxy)borohydride or sodium cyanoborohydride. In one embodiment, the borohydride reagent is sodium tri(acetoxy)borohydride.

In some embodiments, the molar ratio of the compound of Formula (V) to the reducing agent is from about 1:1 to about 1:2. In one embodiment, the molar ratio of the compound of Formula (V) to reducing agent is about 1:1.5.

In some embodiments, step 2.0 occurs in the presence of a catalyst. In some embodiments, step 2.0 occurs in the presence of an acid catalyst. In some embodiments, step 2.0 occurs in the presence of a Lewis acid catalyst. In some embodiments, the Lewis acid catalyst is titanium tetra(isopropoxide) or zinc dichloride. In other embodiments, step 2.0 occurs in the presence of a Bronsted acid catalyst. In some embodiments, the Bronsted acid catalyst is an organic acid. In some embodiments, the organic acid is a carboxylic acid of the form R^{b}COOH wherein R^{b} is hydrogen, substituted or unsubstituted C₁₋₁₀ alkyl, substituted or unsubstituted C₁₋₁₀ haloalkyl, or substituted or unsubstituted C₅₋₁₄ aryl. In some embodiments, the Bronsted acid catalyst is formic acid, acetic acid, trifluoroacetic acid, or benzoic acid. In one embodiment, step 2.0 occurs in the presence of trifluoroacetic acid.

In some embodiments, the molar ratio of the compound of Formula (V) to the catalyst is from about 1:1 to about 1:5. In one embodiment, the molar ratio of the compound of Formula (V) to catalyst is about 1:3.

Step 2.0 may occur in a solvent suitable for the reaction. In one embodiment, the solvent is acetonitrile.

In one embodiment, the compound of Formula (V) is reacted with a bis-hydrochloride salt of the compound of Formula (III) and sodium tri(acetoxy)borohydride in the presence of trifluoroacetic acid as a catalyst, and the molar ratio of the compound of Formula (V) to the compound of Formula (III) is about 1:1.2.

In one exemplary embodiment, the compound of Formula (V) is reacted with a bis-oxalic acid salt of the compound of Formula (III) and sodium tri(acetoxy)borohydride in the presence of trifluoroacetic acid as a catalyst, and the molar ratio of the compound of Formula (V) to the compound of Formula (III) is about 1:1.2.

In some embodiments, provided herein are processes for the preparation of a compound of Formula (III), or a salt, solvate, hydrate, or isotopologue thereof, comprising:

### (Step 3.0) Reacting a Compound of Formula (IV):

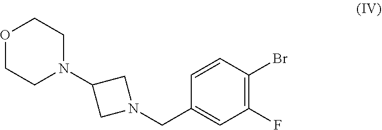 [SMILES: Fc1cc(CN2CC(N3CCOCC3)C2)ccc1Br]

or a salt, solvate, hydrate, or isotopologue thereof, with a formaldehyde source.

In some embodiments, a salt of a compound of Formula (IV) is first converted to the free base form of the compound of Formula (IV) before reacting with a formaldehyde source. In some embodiments, the free base form of the compound of Formula (IV) is formed by contacting the salt of the compound of Formula (IV) with a basic aqueous solution and, optionally, an organic solvent. In some embodiments, the free base form of the compound of Formula (IV) is formed in situ by contacting the salt of the compound of Formula (IV) with a basic aqueous solution, and then reacts with a formaldehyde source without isolation. In some embodiments, the free base form of the compound of Formula (IV) is purified and/or isolated before reacting with a formaldehyde source. In some embodiments, the basic aqueous solution is an aqueous sodium hydroxide solution. In some embodiments, the molar ratio of the compound of Formula (IV) to sodium hydroxide is about 1:2.8. In some embodiments, the organic solvent is methyl tert-butyl ether.

In one embodiment, the salt of a compound of Formula (IV) is a methanesulfonic acid salt. In one embodiment, the salt is a bis-methanesulfonic acid salt.

Step 3.0 may occur in the presence of any formaldehyde source suitable for the reaction. In some embodiments, the formaldehyde source is paraformaldehyde, 1,3,5-trioxane or dimethylformamide (DMF). In one embodiment, the formaldehyde source is dimethylformamide (DMF).

In some embodiments, the molar ratio of the compound of Formula (IV) to the formaldehyde source is from about 1:1 to about 1:3. In one embodiment, the molar ratio of the compound of Formula (IV) to formaldehyde source is about 1:1.9.

In some embodiments, step 3.0 occurs in the presence of an organometallic reagent. In some embodiments, step 3.0 occurs in the presence of an organolithium, organomagnesium or organozinc reagent. In some embodiments, step 3.0 occurs in the presence of an organomagnesium reagent. In one embodiment, the organomagnesium reagent is iPrMgCl·LiCl.

In some embodiments, the molar ratio of the compound of Formula (IV) to the organometallic reagent is from about 1:1 to about 1:2. In one embodiment, the molar ratio of the compound of Formula (IV) to organometallic reagent is about 1:1.6.

In some embodiments, the compound of Formula (IV) is converted to an organometallic reagent in step 3.0. In some embodiments, the organometallic reagent is formed in situ, or is isolated therefrom. In some embodiments, the compound of Formula (IV) is converted to an organolithium, organomagnesium or organozinc reagent. In some embodiments, the compound of Formula (IV) is converted to an organomagnesium reagent. In some embodiments, the organomagnesium reagent is formed by contacting a compound of Formula (IV) with a form of magnesium metal and, optionally, a catalyst. In another embodiment, the organomagnesium reagent is formed by contacting a compound of Formula (IV) with iPrMgCl·LiCl.

Step 3.0 may occur in a solvent suitable for the reaction. In one embodiment, the solvent is tetrahydrofuran (THF), methyl tert-butyl ether (MTBE), or dimethylformamide (DMF), or a mixture thereof. In another embodiment, the solvent is tetrahydrofuran.

In some embodiments, step 3.0 occurs at a reaction temperature of from about −30 to about 10° C. In one embodiment, the reaction temperature is about −20° C.

In some embodiments, the compound of Formula (III) formed in step 3.0 is converted to a salt of the compound. In one embodiment, the salt is a hydrochloride salt. In one embodiment, the salt is a bis-hydrochloride salt. In some embodiments, the salt is formed by reacting a compound of Formula (III) with hydrochloric acid. In one embodiment, a compound of Formula (III) is reacted with hydrochloric acid in a solvent of a mixture of methyltetrahydrofuran, isopropyl alcohol (IPA) and water.

In some embodiments, the process further comprises:
(step 3.a) reacting the compound of Formula (III), or a salt, solvate, hydrate, or isotopologue thereof, prepared in step 3.0 with Na₂S₂O₅ to provide a sodium sulfonate compound of the Formula:

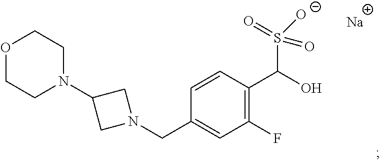 [SMILES: O=S(=O)([O-])C(O)c1ccc(CN2CC(N3CCOCC3)C2)cc1F ; [Na+]]

or a salt, solvate, hydrate, or isotopologue thereof, and
(step 3.b) converting the sodium sulfonate compound to the compound of Formula (III), or a salt, solvate, hydrate, or isotopologue thereof.

In some embodiments, the free base form of a compound of Formula (III) is isolated from step 3.0 and is then subsequently reacted with Na₂S₂O₅ in step 3.a. In some embodiments, Na₂S₂O₅ is added as a solution in a protic solvent. In one embodiment, Na₂S₂O₅ is added as a solution in ethanol or water, or a combination thereof. In other embodiments, Na₂S₂O₅ is added as a solid.

In some embodiments, step 3.b occurs in the presence of base. In some embodiments, step 3.b occurs in the presence of an alkali metal base. In some embodiments, the base is an alkali metal hydroxide, carbonate, hydrogencarbonate, phosphate, hydrogenphosphate, or dihydrogenphosphate. In some embodiments, the base is LiOH, NaOH, KOH, Na₂CO₃, K₂CO₃, Cs₂CO₃, NaHCO₃, KHCO₃, Na₃PO₄, K₃PO₄, Na₂HPO₄, K₂HPO₄, NaH₂PO₄, or KH₂PO₄. In one embodiment, the base is sodium carbonate (Na₂CO₃).

Step 3.b may occur in a solvent suitable for the reaction. In one embodiment, the solvent is a mixture of ethyl acetate (EtOAc) or water, or a mixture thereof.

In some embodiments, the compound of Formula (III) formed in step 3.b is converted to a salt of the compound. In one embodiment, the salt is an oxalic acid salt. In one embodiment, the salt is a bis-oxalic acid salt. In some embodiments, the salt is formed by reacting a compound of Formula (III) with oxalic acid. In one embodiment, a compound of Formula (III) is reacted with oxalic acid in a solvent of isopropyl alcohol (IPA) or water, or a mixture thereof.

In one embodiment, a compound of Formula (IV) is reacted with dimethylformamide in the presence of iPrMgCl·LiCl, in a solvent of tetrahydrofuran; the free base form of a compound of Formula (III) is isolated; a solution of Na₂S₂O₅ in ethanol and water is then added; the sodium sulfonate compound is then reacted with sodium carbonate in a solvent of a mixture of ethyl acetate and water. In one embodiment, the compound of Formula (III) is converted to a bis-oxalic acid salt by treating the compound of Formula (III) with oxalic acid in a solvent of a mixture of isopropyl alcohol (IPA) and water.

In some embodiments, provided herein are processes for the preparation of a compound of Formula (IV), or a salt, solvate, hydrate, or isotopologue thereof, comprising:
(step 4.0) reacting 4-(azetidin-3-yl)morpholine, or a salt thereof, with 4-bromo-3-fluorobenzaldehyde.

In some embodiments, a salt of 4-(azetidin-3-yl)morpholine is used in step 4.0. In one embodiment, a hydrochloride salt of 4-(azetidin-3-yl)morpholine is used. In one embodiment, the molar ratio of 4-bromo-3-fluorobenzaldehyde to 4-(azetidin-3-yl)morpholine hydrochloride is about 1:1.

In some embodiments, step 4.0 occurs in the presence of a reducing agent. In some embodiments, the reducing agent is a borohydride reagent. In some embodiments, the borohydride reagent is sodium borohydride, sodium tri(acetoxy)borohydride or sodium cyanoborohydride. In one embodiment, the borohydride reagent is sodium tri(acetoxy)borohydride. In one embodiment, the molar ratio of 4-bromo-3-fluorobenzaldehyde to sodium tri(acetoxy)borohydride is about 1:1.7.

In some embodiments, step 4.0 occurs in the presence of a catalyst. In some embodiments, step 4.0 occurs in the presence of an acid catalyst. In some embodiments, step 4.0 occurs in the presence of a Lewis acid catalyst. In some embodiments, the Lewis acid catalyst is titanium tetra(isopropoxide) or zinc dichloride. In other embodiments, step 4.0 occurs in the presence of a Bronsted acid catalyst. In some embodiments, the Bronsted acid catalyst is an organic acid. In some embodiments, the Bronsted acid catalyst is formic acid, acetic acid, trifluoroacetic acid, or benzoic acid. In one embodiment, the hydrochloride salt of 4-(azetidin-3-yl)morpholine is the acid source.

Step 4.0 may occur in a solvent suitable for the reaction. In one embodiment, the solvent is acetonitrile.

In some embodiments, the compound of Formula (IV) formed in step 4.0 is converted to a salt of the compound. In one embodiment, the salt is a citric acid salt. In one embodiment, the salt is a citric acid salt is a bis-citric acid salt. In one embodiment, the salt is a methanesulfonic acid salt. In one embodiment, the methanesulfonic acid salt is a bis-methanesulfonic acid salt. In one embodiment, a citric acid salt of the compound of Formula (IV) is converted to a methanesulfonic acid salt of the compound of Formula (IV) in step 4.0.

In some embodiments, in step 4.0 a citric acid salt of the compound of Formula (IV) is formed by reacting a compound of Formula (IV) with citric acid. In one embodiment, a compound of Formula (IV) is reacted with citric acid in a solvent of cyclopentyl methyl ether.

In some embodiments, in step 4.0 a methanesulfonic acid salt of the compound of Formula (IV) is formed by treating a citric acid salt of the compound of Formula (IV) with a basic aqueous solution followed acidification with methanesulfonic acid. In some embodiments, the citric acid salt of the compound of Formula (IV) is treated with an aqueous solution of sodium hydroxide, optionally in the presence of a solvent of cyclopentyl methyl ether. In some embodiments, acidification with methanesulfonic acid occurs in the presence a solvent of methanol or cyclopentyl methyl ether, or a mixture thereof.

In one embodiment, 4-bromo-3-fluorobenzaldehyde is reacted with a hydrochloride salt of 4-(azetidin-3-yl)morpholine and sodium tri(acetoxy)borohydride; and the compound of Formula (IV) is optionally converted first to a citric acid salt of the compound, followed by conversion of the citric acid salt to a methanesulfonic acid salt of the compound.

In some embodiments, provided herein are processes for the preparation of a compound of Formula (V), or a salt, solvate, hydrate, enantiomer, mixture of enantiomers, or isotopologue thereof, comprising:
(step 5.0) reducing a compound of Formula (VI):

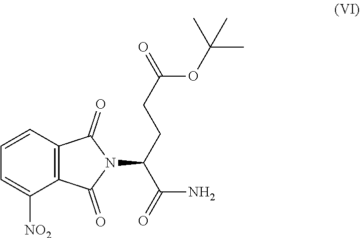 [SMILES: CC(C)(C)OC(=O)CC[C@@H](C(N)=O)N1C(=O)c2cccc([N+](=O)[O-])c2C1=O]

or a salt, solvate, hydrate, enantiomer, mixture of enantiomers, or isotopologue thereof.

In some embodiments, step 5.0 occurs under a hydrogenation condition. In one embodiment, the hydrogenation occurs in the presence of hydrogen gas. In other embodiments, the hydrogenation occurs under a transfer hydrogenation condition. In some embodiments, the transfer hydrogenation condition includes cyclohexene, cyclohexadiene, formic acid, or ammonium formate.

In some embodiments, step 5.0 occurs in the presence of a palladium, platinum, rhodium, or ruthenium catalyst on different supports that include carbons, alumina, alkaline earth carbonates, clays, ceramics, or celite. In some embodiments, the hydrogenation occurs in the presence of a palladium catalyst. In one embodiment, the catalyst is palladium on carbon (Pd/C).

Step 5.0 may occur in a solvent suitable for the reaction. In one embodiment, the solvent is isopropyl alcohol (IPA).

In one exemplary embodiment, a compound of Formula (VI) is reacted with hydrogen gas in the presence of palladium on carbon as a catalyst.

In some embodiments, provided herein are processes for the preparation of a compound of Formula (VI), or a salt, solvate, hydrate, enantiomer, mixture of enantiomers, or isotopologue thereof, comprising:
(step 6.0) reacting (S)-tert-butyl 4,5-diamino-5-oxopentanoate of the Formula:

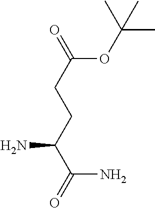

or a salt, solvate, hydrate, enantiomer, mixture of enantiomers, or isotopologue thereof, with 3-nitrophthalic anhydride.

In some embodiments, a salt of (S)-tert-butyl 4,5-diamino-5-oxopentanoate is used in step 6.0. In one embodiment a hydrochloride salt of (S)-tert-butyl 4,5-diamino-5-oxopentanoate is used.

In some embodiments, step 6.0 occurs in the presence of base. In some embodiments, the base is a nitrogen containing base. In some embodiments, the base is NH₄OH, triethylamine, diisopropylethylamine (DIEA), pyridine, lutidine, 4-dimethylaminopyridine, imidazole, or 1,8-diazabicyclo[5.4.0]undec-7-ene (DBU). In one embodiment, the base is lutidine. In one embodiment, the lutidine is 2,3-lutidine, 2,4-lutidine, 2,5-lutidine, 2,6-lutidine, 3,4-lutidine, or 3,5-lutidine, or a mixture thereof.

In some embodiments, step 6.0 occurs in the presence of an activating reagent. In one embodiment, the activating reagent is 1,1′-carbonyldiimidazole (CDI).

Step 6.0 may occur in a solvent suitable for the reaction. In one embodiment, the solvent is a mixture of dimethylformamide (DMF), ethyl acetate (EtOAc), and methyltetrahydrofuran.

In one embodiment, a hydrochloride salt of (S)-tert-butyl 4,5-diamino-5-oxopentanoate is reacted with 3-nitrophthalic anhydride in the presence of lutidine as a base and 1,1′-carbonyldiimidazole as an activating reagent.

In some embodiments, provided herein are processes for the preparation of a compound of Formula (VI), or a salt, solvate, hydrate, enantiomer, mixture of enantiomers, or isotopologue thereof, comprising:
(step 6.a) reacting (S)-tert-butyl 4,5-diamino-5-oxopentanoate of the Formula:

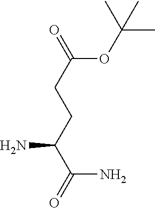

or a salt, solvate, hydrate, enantiomer, mixture of enantiomers, or isotopologue thereof, with ethyl 4-nitro-1,3-dioxoisoindoline-2-carboxylate of the Formula:

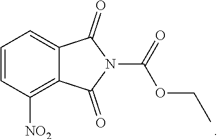 [SMILES: CCOC(=O)N1C(=O)c2cccc([N+](=O)[O-])c2C1=O]

In some embodiments, a salt of (S)-tert-butyl 4,5-diamino-5-oxopentanoate is used in step 6.a. In one embodiment, a hydrochloride salt of (S)-tert-butyl 4,5-diamino-5-oxopentanoate is used.

In some embodiments, the molar ratio of (S)-tert-butyl 4,5-diamino-5-oxopentanoate to ethyl 4-nitro-1,3-dioxoisoindoline-2-carboxylate is from about 1:2 to 2:1. In one embodiment, the molar ratio of (S)-tert-butyl 4,5-diamino-5-oxopentanoate to ethyl 4-nitro-1,3-dioxoisoindoline-2-carboxylate is about 1:1.

In some embodiments, step 6.a occurs in the presence of base. In some embodiments, the base is a nitrogen containing base. In some embodiments, the base is NH₄OH, triethylamine, diisopropylethylamine (DIEA), pyridine, lutidine, 4-dimethylaminopyridine, imidazole, or 1,8-diazabicyclo[5.4.0]undec-7-ene (DBU). In one embodiment, the base is diisopropylethylamine (DIEA).

In some embodiments, the molar ratio of (S)-tert-butyl 4,5-diamino-5-oxopentanoate to base is from about 1:1 to 1:2. In one embodiment, the molar ratio of (S)-tert-butyl 4,5-diamino-5-oxopentanoate to base is about 1:1.4.

Step 6.a may occur in a solvent suitable for the reaction. In one embodiment, the solvent is tetrahydrofuran.

In some embodiments, step 6.a occurs at a reaction temperature of from about 60° C. to about 80° C. In one embodiment, the reaction temperature is about 68° C.

In some embodiments, step 6.a occurs at a reaction time of from about 6 hours to about 18 hours. In one embodiment, the reaction time is about 10 hours.

In one exemplary embodiment, (S)-tert-butyl 4,5-diamino-5-oxopentanoate is reacted with ethyl 4-nitro-1,3-dioxoisoindoline-2-carboxylate in the presence of diisopropylethylamine as a base, the molar ratio of (S)-tert-butyl 4,5-diamino-5-oxopentanoate to ethyl 4-nitro-1,3-dioxoisoindoline-2-carboxylate is about 1:1, the molar ratio of (S)-tert-butyl 4,5-diamino-5-oxopentanoate to diisopropylethylamine is about 1:1.4, the solvent is tetrahydrofuran. In one embodiment, the reaction temperature is about 68° C., and the reaction time is about 10 hours. In one embodiment, the compound of Formula (VI) is purified by precipitation with methyl tert-butyl ether, extraction into dichloromethane, and trituration with a mixture of hexane and ethyl acetate.

In some embodiments, provided herein are processes for the preparation of ethyl 4-nitro-1,3-dioxoisoindoline-2-carboxylate, comprising:
(step 6.b) reacting 4-nitroisoindoline-1,3-dione with ethyl chloroformate.

In some embodiments, the molar ratio of 4-nitroisoindoline-1,3-dione to ethyl chloroformate is from about 2:1 to about 1:2. In one embodiment, the molar ratio of 4-nitroisoindoline-1,3-dione to ethyl chloroformate is about 1:1.25.

In some embodiments, step 6.b occurs in the presence of base. In some embodiments, the base is a nitrogen containing base. In some embodiments, the base is NH₄OH, triethylamine, diisopropylethylamine (DIEA), pyridine, lutidine, 4-dimethylaminopyridine, imidazole, or 1,8-diazabicyclo[5.4.0]undec-7-ene (DBU). In one embodiment, the base is trimethylamine (TEA).

In some embodiments, the molar ratio of 4-nitroisoindoline-1,3-dione to base is from about 1:1 to about 1:2. In one embodiment, the molar ratio of 4-nitroisoindoline-1,3-dione to base is about 1:1.13.

Step 6.b may occur in a solvent suitable for the reaction. In one embodiment, the solvent dimethylformamide. In one embodiment, the dimethylformamide is anhydrous.

In some embodiments, step 6.b occurs at a reaction temperature of from about 0° C. to about 30° C. In one embodiment, the reaction temperature is about 22° C.

In some embodiments, step 6.b occurs at a reaction time of from about 6 hours to about 18 hours. In one embodiment, the reaction time is about 10 hours.

In one embodiment, 4-nitroisoindoline-1,3-dione is reacted with ethyl chloroformate in the presence of diisopropylethylamine as a base, the molar ratio of 4-nitroisoindoline-1,3-dione to ethyl chloroformate is about 1:1.25, the molar ratio of 4-nitroisoindoline-1,3-dione to diisopropylethylamine is about 1:1.13, and the solvent is dimethylformamide. In one embodiment, the reaction temperature is about 22° C., and the reaction time is about 10 hours. In one embodiment, 4-nitro-1,3-dioxoisoindoline-2-carboxylate is optionally purified by filtration followed by selective extraction into ethyl acetate.

In certain embodiments, the processes provided herein result in improved chiral purity for one or more intermediates and/or products throughout the route.

In certain embodiments, the processes provided herein result in improved impurity profiles for one or more intermediates and/or products throughout the route.

In certain embodiments, the processes provided herein result in a more convergent synthesis for one or more intermediates and/or products throughout the route.

All of the combinations of the above embodiments are encompassed by this invention.

In one embodiment, provided herein is a process for the preparation of a compound of Formula (I), or a salt, solvate, hydrate, enantiomer, mixture of enantiomers, or isotopologue thereof, comprising:
(step 1.0) cyclizing a compound of Formula (II), or a salt, solvate, hydrate, enantiomer, mixture of enantiomers, or isotopologue thereof, to provide a compound of Formula (I), or a salt, solvate, hydrate, enantiomer, mixture of enantiomers, or isotopologue thereof; and
(step 1.1) optionally converting the compound of Formula (I), or a salt, solvate, hydrate, enantiomer, mixture of enantiomers, or isotopologue thereof, to a salt of the compound;
   wherein the compound of Formula (II), or a salt, solvate, hydrate, enantiomer, mixture of enantiomers, or isotopologue thereof, is prepared by a process comprising:
(step 2.0) reacting a compound of Formula (III), or a salt, solvate, hydrate, or isotopologue thereof, with a compound of Formula (V) or a salt, solvate, hydrate, enantiomer, mixture of enantiomers, or isotopologue thereof;
   wherein the compound of Formula (III), or a salt, solvate, hydrate, or isotopologue thereof, is prepared by a process comprising:
(step 3.0) reacting a compound of Formula (IV), or a salt, solvate, hydrate, or isotopologue thereof, with a formaldehyde source;
   wherein the compound of Formula (IV), or a salt, solvate, hydrate, or isotopologue thereof, is prepared by a process comprising:
(step 4.0) reacting 4-(azetidin-3-yl)morpholine, or a salt thereof, with 4-bromo-3-fluorobenzaldehyde;
   wherein the compound of Formula (V), or a salt, solvate, hydrate, enantiomer, mixture of enantiomers, or isotopologue thereof, is prepared by a process comprising:
(step 5.0) reducing a compound of Formula (VI), or a salt, solvate, hydrate, enantiomer, mixture of enantiomers, or isotopologue thereof; and
   wherein the compound of Formula (VI), or a salt, solvate, hydrate, enantiomer, mixture of enantiomers, or isotopologue thereof, is prepared by a process comprising:
(step 6.0) reacting (S)-tert-butyl 4,5-diamino-5-oxopentanoate or a salt, solvate, hydrate, enantiomer, mixture of enantiomers, or isotopologue thereof, with 3-nitrophthalic anhydride.

In another embodiment, provided herein is a process for the preparation of a compound of Formula (I), or a salt, solvate, hydrate, enantiomer, mixture of enantiomers, or isotopologue thereof, by a process comprising:
(step 1.0) cyclizing a compound of Formula (II), or a salt, solvate, hydrate, enantiomer, mixture of enantiomers, or isotopologue thereof, to provide a compound of Formula (I), or a salt, solvate, hydrate, enantiomer, mixture of enantiomers, or isotopologue thereof; and
(step 1.1) optionally converting the compound of Formula (I), or a salt, solvate, hydrate, enantiomer, mixture of enantiomers, or isotopologue thereof, to a salt of the compound;
   wherein the compound of Formula (II), or a salt, solvate, hydrate, enantiomer, mixture of enantiomers, or isotopologue thereof, is prepared by a process comprising:
(step 2.0) reacting a compound of Formula (III), or a salt, solvate, hydrate, or isotopologue thereof, with a compound of Formula (V), or a salt, solvate, hydrate, enantiomer, mixture of enantiomers, or isotopologue thereof;
   wherein the compound of Formula (III), or a salt, solvate, hydrate, or isotopologue thereof, is prepared by a process comprising:
(step 3.0) reacting a compound of Formula (IV), or a salt, solvate, hydrate, or isotopologue thereof, with a formaldehyde source;
(step 3.a) reacting the compound of Formula (III), or a salt, solvate, hydrate, or isotopologue thereof, prepared in step 3.0 with Na₂S₂O₅ to provide a sodium sulfonate compound, or a salt, solvate, hydrate, or isotopologue thereof, and
(step 3.b) converting the sodium sulfonate compound to the compound of Formula (IV), or a salt, solvate, hydrate, or isotopologue thereof;
   wherein the compound of Formula (IV), or a salt, solvate, hydrate, enantiomer, mixture of enantiomers, or isotopologue thereof, is prepared by a process comprising:
(step 4.0) reacting 4-(azetidin-3-yl)morpholine, or a salt thereof, with 4-bromo-3-fluorobenzaldehyde;
   wherein the compound of Formula (V) or a salt, solvate, hydrate, enantiomer, mixture of enantiomers, or isotopologue thereof, is prepared by a process comprising:
(step 5.0) reducing a compound of Formula (VI) or a salt, solvate, hydrate, enantiomer, mixture of enantiomers, or isotopologue thereof; and
   wherein the compound of Formula (VI) or a salt, solvate, hydrate, enantiomer, mixture of enantiomers, or isotopologue thereof, is prepared by a process comprising:
(step 6.0) reacting (S)-tert-butyl 4,5-diamino-5-oxopentanoate or a salt, solvate, hydrate, enantiomer, mixture of enantiomers, or isotopologue thereof, with 3-nitrophthalic anhydride.

In another embodiment, provided herein is a process for the preparation of a compound of Formula (I), or a salt, solvate, hydrate, enantiomer, mixture of enantiomers, or isotopologue thereof, by a process comprising:
(step 1.0) cyclizing a compound of Formula (II), or a salt, solvate, hydrate, enantiomer, mixture of enantiomers, or isotopologue thereof, to provide a compound of Formula (I), or a salt, solvate, hydrate, enantiomer, mixture of enantiomers, or isotopologue thereof; and
(step 1.1) optionally converting the compound of Formula (I), or a salt, solvate, hydrate, enantiomer, mixture of enantiomers, or isotopologue thereof, to a salt of the compound;
   wherein the compound of Formula (II), or a salt, solvate, hydrate, enantiomer, mixture of enantiomers, or isotopologue thereof, is prepared by a process comprising:
(step 2.a) reacting a compound of Formula (II-A), or a salt, solvate, hydrate, enantiomer, mixture of enantiomers, or isotopologue thereof, with 4-(azetidin-3-yl)morpholine, or a salt thereof;
   wherein the compound of Formula (II-A), or a salt, solvate, hydrate, enantiomer, mixture of enantiomers, or isotopologue thereof, is prepared by a process comprising:
(step 2.b) chlorinating a compound of Formula (II-B), or a salt, solvate, hydrate, enantiomer, mixture of enantiomers, or isotopologue thereof;
   wherein the compound of Formula (II-B), or a salt, solvate, hydrate, enantiomer, mixture of enantiomers, or isotopologue thereof, is prepared by a process comprising:
(step 2.c) reacting a compound of Formula (V), or a salt, solvate, hydrate, enantiomer, mixture of enantiomers, or isotopologue thereof, with 2-fluoro-4-(hydroxymethyl)benzaldehyde;
   wherein the compound of Formula (V), or a salt, solvate, hydrate, enantiomer, mixture of enantiomers, or isotopologue thereof, is prepared by a process comprising:
(step 5.0) reducing a compound of Formula (VI), or a salt, solvate, hydrate, enantiomer, mixture of enantiomers, or isotopologue thereof; and
   wherein the compound of Formula (VI), or a salt, solvate, hydrate, enantiomer, mixture of enantiomers, or isotopologue thereof, is prepared by a process comprising:
(step 6.a) reacting (S)-tert-butyl 4,5-diamino-5-oxopentanoate, or a salt, solvate, hydrate, enantiomer, mixture of enantiomers, or isotopologue thereof, with ethyl 4-nitro-1,3-dioxoisoindoline-2-carboxylate; and
   wherein ethyl 4-nitro-1,3-dioxoisoindoline-2-carboxylateis prepared by a process comprising:
(step 6.b) reacting 4-nitroisoindoline-1,3-dione with ethyl chloroformate.

### 6.3 Compounds and Solid Forms

In one embodiment, provided herein are intermediate compounds used in or product compounds prepared by the processes provided herein, including solid forms (e.g., crystalline forms) thereof.

In one embodiment, provided herein is a bis-besylate salt of Compound 1:

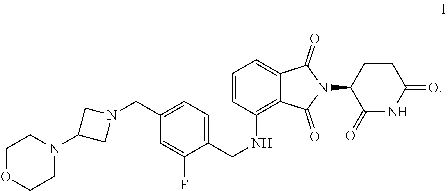 [SMILES: O=C1CC[C@H](N2C(=O)c3cccc(NCc4ccc(CN5CC(N6CCOCC6)C5)cc4F)c3C2=O)C(=O)N1]

In one embodiment, provided herein are solid forms (e.g., Form B) comprising a besylate salt of Compound 1. Certain salts and solid forms of Compound 1 (including Form A of hydrochloride salt of Compound 1 and Form A of besylate salt of Compound 1) are described in U.S. Patent Application Publication No. 2021-0115019, the entirety of which is incorporated herein by reference.

In one embodiment, provided herein is Compound 2:

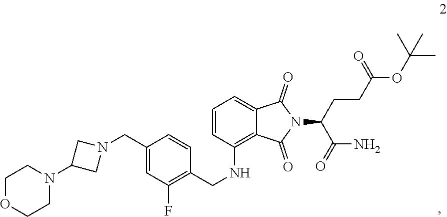 [SMILES: CC(C)(C)OC(=O)CC[C@@H](C(N)=O)N1C(=O)c2cccc(NCc3ccc(CN4CC(N5CCOCC5)C4)cc3F)c2C1=O]

or a salt, solvate, hydrate, enantiomer, mixture of enantiomers, or isotopologue thereof.

In one embodiment, provided herein is Compound 2-a:

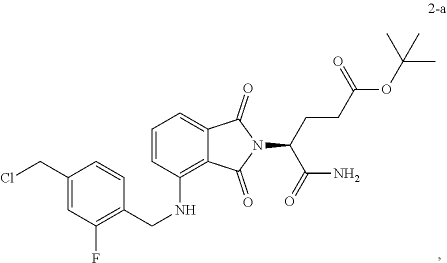 [SMILES: CC(C)(C)OC(=O)CC[C@@H](C(N)=O)N1C(=O)c2cccc(NCc3ccc(CCl)cc3F)c2C1=O]

or a salt, solvate, hydrate, enantiomer, mixture of enantiomers, or isotopologue thereof.

In one embodiment, provided herein is Compound 2-b:

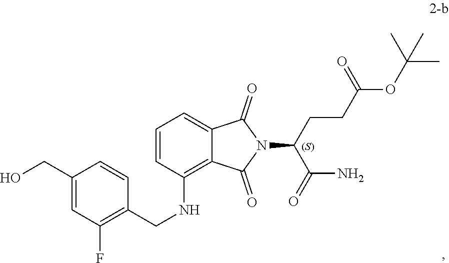

or a salt, solvate, hydrate, enantiomer, mixture of enantiomers, or isotopologue thereof.

In one embodiment, provided herein is Compound 3:

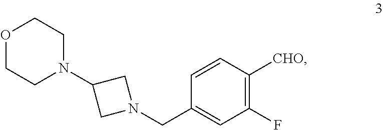 [SMILES: O=Cc1ccc(CN2CC(N3CCOCC3)C2)cc1F]

or a salt, solvate, hydrate, or isotopologue thereof.

In one embodiment, provided herein is a salt of Compound 3. In one embodiment, the salt is a hydrochloride salt. In one embodiment, the hydrochloride salt is a dihydrochloride salt. In one embodiment, provided herein are solid forms (e.g., Form A or Form B) comprising a hydrochloride salt of Compound 3. In one embodiment, the salt is an oxalic acid salt. In one embodiment, the oxalic acid salt is a bis-oxalic acid salt.

In one embodiment, provided herein is Compound 4:

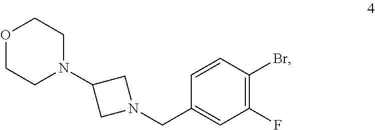 [SMILES: Fc1cc(CN2CC(N3CCOCC3)C2)ccc1Br]

or a salt, solvate, hydrate, or isotopologue thereof.

In one embodiment, provided herein is a salt of Compound 4. In one embodiment, the salt is a methanesulfonic acid salt. In one embodiment, the methanesulfonic acid salt is a bis-methanesulfonic acid salt. In one embodiment, provided herein are solid forms (e.g., Form A) comprising a methanesulfonic acid salt of Compound 4.

In one embodiment, provided herein is Compound 5:

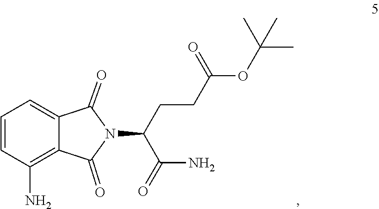 [SMILES: CC(C)(C)OC(=O)CC[C@@H](C(N)=O)N1C(=O)c2cccc(N)c2C1=O]

or a salt, solvate, hydrate, enantiomer, mixture of enantiomers, or isotopologue thereof.

In one embodiment, provided herein is Compound 6:

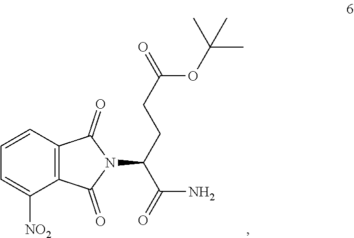 [SMILES: CC(C)(C)OC(=O)CC[C@@H](C(N)=O)N1C(=O)c2cccc([N+](=O)[O-])c2C1=O]

or a salt, solvate, hydrate, enantiomer, mixture of enantiomers, or isotopologue thereof.

### 5.3.1 Form B of Besylate Salt of Compound 1

In one embodiment, provided herein is a solid form comprising a besylate salt of Compound 1:

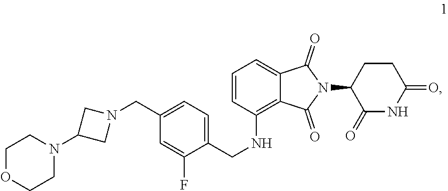 [SMILES: O=C1CC[C@H](N2C(=O)c3cccc(NCc4ccc(CN5CC(N6CCOCC6)C5)cc4F)c3C2=O)C(=O)N1]

wherein the solid form is Form B (of a besylate salt of Compound 1).

In some embodiments, the molar ratio of Compound 1 to benzenesulfonic acid in the solid form ranges from about 1:1 to about 1:2. In one embodiment, the molar ratio is about 1:2 (i.e., bis-besylate salt).

In one embodiment, Form B is crystalline. In one embodiment, Form B is substantially crystalline. In one embodiment, Form B is moderately crystalline. In one embodiment, Form B is partially crystalline.

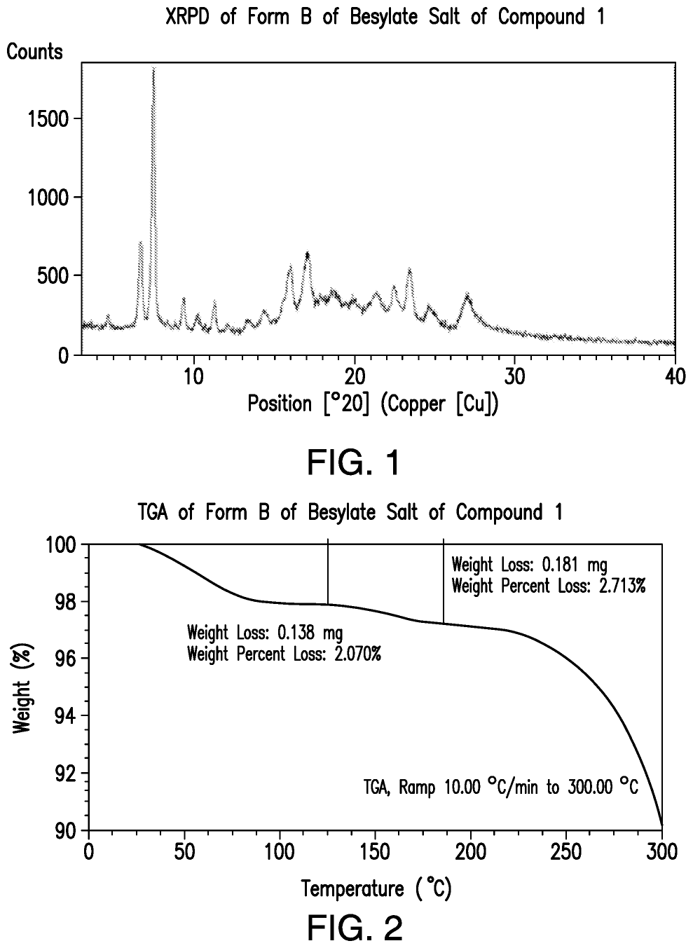

A representative XRPD pattern of the Form B of a besylate salt of Compound 1 is provided in FIG. 1.

In one embodiment, provided herein is a solid form comprising a besylate salt of Compound 1, characterized by 1, 2, 3, 4, 5, 6, 7, 8, 9, 10, 11, 12, 13, 14, 15, 16, 17 or all of the XRPD peaks located at approximately the following positions: 4.7, 6.7, 7.5, 9.4, 10.2, 11.3, 12.1, 13.4, 14.3, 16.0, 17.2, 18.6, 19.9, 21.4, 22.4, 23.5, 24.6, and 26.9° 2θ. In one embodiment, the solid form is characterized by 3 of the peaks. In one embodiment, the solid form is characterized by 5 of the peaks. In one embodiment, the solid form is characterized by 7 of the peaks. In one embodiment, the solid form is characterized by 9 of the peaks. In one embodiment, the solid form is characterized by 11 of the peaks. In one embodiment, the solid form is characterized by all of the peaks.

In one embodiment, provided herein is a solid form comprising a besylate salt of Compound 1, characterized by an XRPD pattern comprising peaks at approximately 6.7, 7.5, and 17.2° 2θ. In one embodiment, the XRPD pattern further comprises peaks at approximately 16.0 and 23.5° 2θ. In one embodiment, the XRPD pattern further comprises peaks at approximately 9.4 and 11.3° 2θ. In one embodiment, the XRPD pattern comprises peaks at approximately 6.7, 7.5, 9.4, 11.3, 16.0, 17.2, 22.4, 23.5, and 26.9° 2θ.

In one embodiment, provided herein is a solid form comprising a besylate salt of Compound 1, characterized by an XRPD pattern that matches the XRPD pattern presented in FIG. 1.

In one embodiment, the XRPD patterns are obtained using Cu Kα radiation.

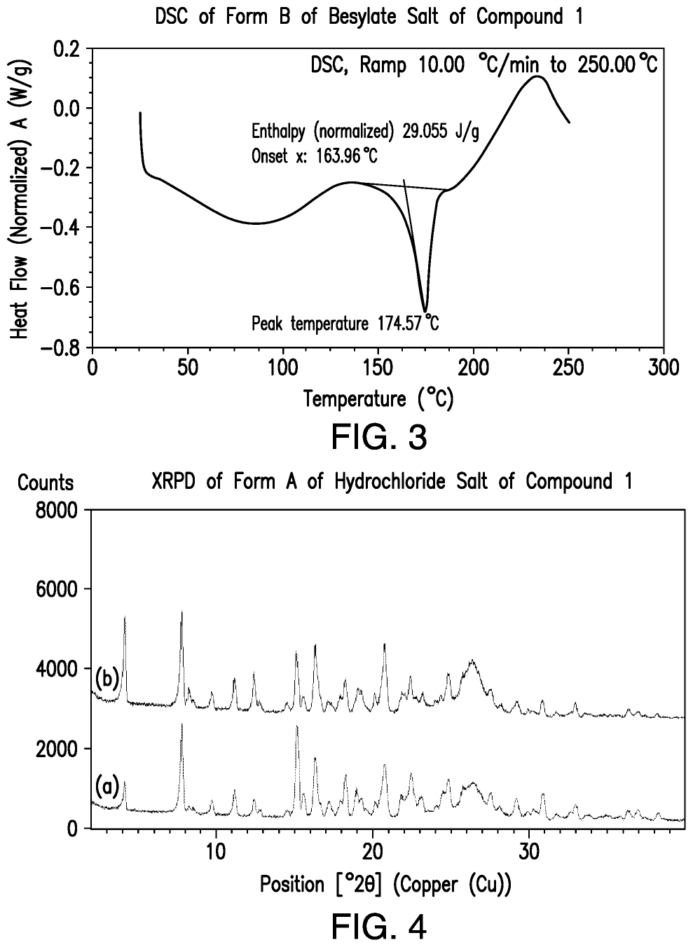

Representative thermal gravimetric analysis (TGA) and differential scanning calorimetry (DSC) thermograms of Form B are provided in FIG. 2 and FIG. 3, respectively. In one embodiment, provided herein is a solid form comprising a besylate salt of Compound 1, which exhibits a weight loss of about 2.1% upon heating from about 25° C. to about 125° C. In one embodiment, provided herein is a solid form comprising a besylate salt of Compound 1, which exhibits a weight loss of about 2.7% upon heating from about 25° C. to about 200° C. In one embodiment, provided herein is a solid form comprising a besylate salt of Compound 1, characterized by a TGA thermogram that matches the TGA thermogram presented in FIG. 2.

In one embodiment, provided herein is a solid form comprising a besylate salt of Compound 1, which exhibits, as characterized by DSC, a thermal event (endo) with an onset temperature of about 164° C. In one embodiment, the thermal even also has a peak temperature of about 175° C. In one embodiment, provided herein is a solid form comprising a besylate salt of Compound 1, characterized by a DSC thermogram that matches the DSC thermogram presented in FIG. 3.

In one embodiment, Form B of a besylate salt of Compound 1 is prepared by (i) adding an anti-solvent to a mixture of a besylate salt of Compound 1 in acetonitrile, resulting in a slurry, and (ii) slurrying the slurry to provide Form B of a besylate salt of Compound. In one embodiment, the anti-solvent is MeTHF. In one embodiment, the anti-solvent is MTBE. In one embodiment, the mixture of a besylate salt of Compound 1 in acetonitrile is formed by adding benzenesulfonic acid to a solution of free base of Compound 1 in acetonitrile (e.g., at about 55° C.). In one embodiment, the solution of free base of Compound 1 in acetonitrile also contains water. In one embodiment of (ii), the slurry is slurried at about 20° C. for a period of time (e.g., from about 1 hour to about 24 hours, e.g., about 6 hours or overnight).

In one embodiment, provided herein is a solid form comprising Form B of a besylate salt of Compound 1 and one or more forms of a free base of Compound 1 (e.g., amorphous form and crystalline forms). In one embodiment, provided herein is a solid form comprising Form B of a besylate salt of Compound 1 and amorphous besylate salt of Compound 1. In one embodiment, provided herein is a solid form comprising Form B of a besylate salt Compound 1 and one or more other crystalline forms of a besylate salt of Compound 1. In one embodiment, provided herein is a solid form comprising Form B of a besylate salt of Compound 1 and one or more forms (e.g., amorphous or crystalline) of a salt of Compound 1 provided herein.

5.3.2 Form A of hydrochloride Salt of Compound 3

In one embodiment, provided herein is a solid form comprising a hydrochloride salt of Compound 3:

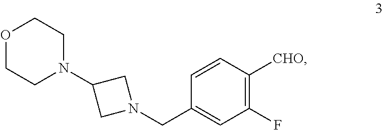 [SMILES: O=Cc1ccc(CN2CC(N3CCOCC3)C2)cc1F]

wherein the solid form is Form A (of the compound of Compound 3).

In some embodiments, the molar ratio of Compound 3 to hydrochloric acid in the solid form ranges from about 1:1 to about 1:2. In one embodiment, the molar ratio is about 1:2 (i.e., dihydrochloride salt).

In one embodiment, Form A is crystalline. In one embodiment, Form A is substantially crystalline. In one embodiment, Form A is moderately crystalline. In one embodiment, Form A is partially crystalline.

In one embodiment, Form A is an anhydrous form (anhydrate) of a hydrochloride salt of Compound 3.

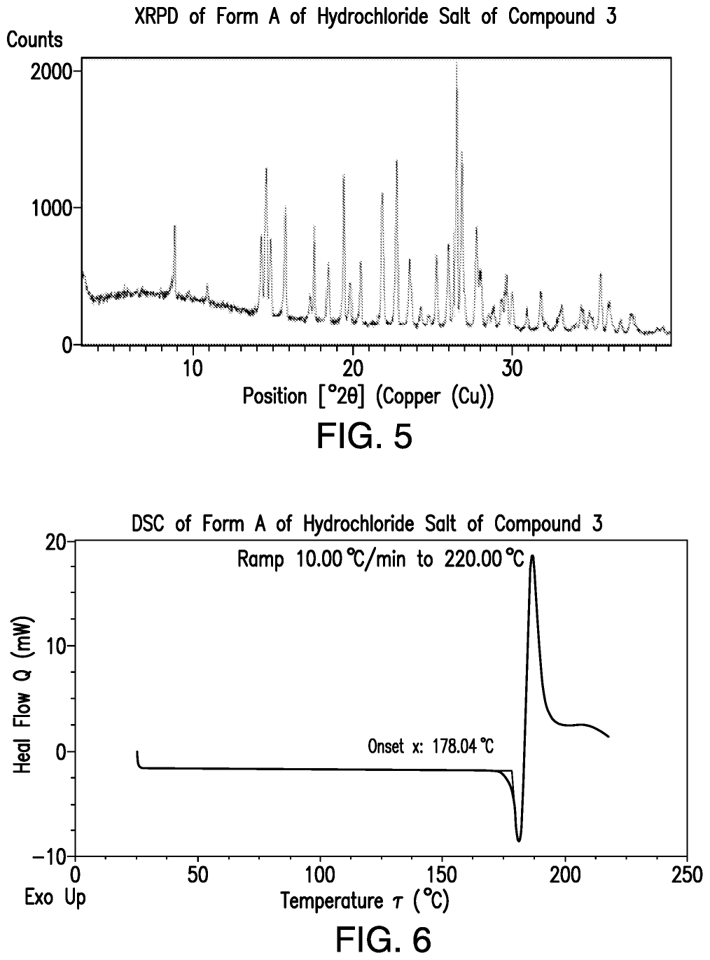

A representative XRPD pattern of the Form A of a hydrochloride salt of Compound 3 is provided in FIG. 5. In one embodiment, provided herein is a solid form comprising a hydrochloride salt of Compound 3, characterized by 1, 2, 3, 4, 5, 6, 7, 8, 9, 10, 11, 12, 13, 14, 15, 16, 17, 18, 19, 20, 21, 22, 23, 24 or all of the XRPD peaks located at approximately the following positions: 8.8, 10.9, 14.3, 14.6, 14.9, 15.8, 17.3, 17.6, 18.4, 19.4, 19.8, 20.5, 21.8, 22.8, 23.5, 24.2, 24.7, 25.2, 26.0, 26.4, 26.8, 27.7, 28.0, 28.4, and 28.8° 2θ. In one embodiment, the solid form is characterized by 3 of the peaks. In one embodiment, the solid form is characterized by 5 of the peaks. In one embodiment, the solid form is characterized by 7 of the peaks. In one embodiment, the solid form is characterized by 9 of the peaks. In one embodiment, the solid form is characterized by 11 of the peaks. In one embodiment, the solid form is characterized by all of the peaks.

In one embodiment, provided herein is a solid form comprising a hydrochloride salt of Compound 3, characterized by an XRPD pattern comprising peaks at approximately 14.6, 19.4, and 21.8° 2θ. In one embodiment, the XRPD pattern further comprises peaks at approximately 15.8 and 22.8° 2θ. In one embodiment, the XRPD pattern further comprises peaks at approximately 8.8, 14.3, and 14.9° 2θ. In one embodiment, the XRPD pattern comprises peaks at approximately 8.8, 14.3, 14.6, 14.9, 15.8, 17.6, 18.4, 19.4, 21.8 and 22.8° 2θ.

In one embodiment, provided herein is a solid form comprising a hydrochloride salt of Compound 3, characterized by an XRPD pattern that matches the XRPD pattern presented in FIG. 5.

In one embodiment, the XRPD patterns are obtained using Cu Kα radiation.

A representative differential scanning calorimetry (DSC) thermogram of Form A is provided in FIG. 6. In one embodiment, provided herein is a solid form comprising a hydrochloride salt of Compound 3, which exhibits, as characterized by DSC, a thermal event (endo) with an onset temperature of about 178° C. In one embodiment, provided herein is a solid form comprising a hydrochloride salt of Compound 3, characterized by a DSC thermogram that matches the DSC thermogram presented in FIG. 6.

In one embodiment, provided herein is a solid form comprising Form A of a hydrochloride salt of Compound 3 and one or more forms of a free base of Compound 3 (e.g., amorphous form and crystalline forms). In one embodiment, provided herein is a solid form comprising Form A of a hydrochloride salt of Compound 3 and amorphous hydrochloride salt of Compound 3. In one embodiment, provided herein is a solid form comprising Form A of a hydrochloride salt Compound 3 and one or more other crystalline forms of a hydrochloride salt of Compound 3. In one embodiment, provided herein is a solid form comprising Form A of a hydrochloride salt of Compound 3 and one or more forms (e.g., amorphous or crystalline) of a salt of Compound 3 provided herein.

5.3.3 Form B of hydrochloride Salt of Compound 3

In one embodiment, provided herein is a solid form comprising a hydrochloride salt of Compound 3:

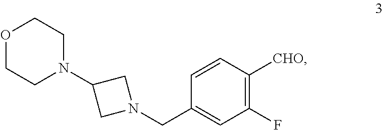 [SMILES: O=Cc1ccc(CN2CC(N3CCOCC3)C2)cc1F]

wherein the solid form is Form B (of the compound of Compound 3).

In some embodiments, the molar ratio of Compound 3 to hydrochloric acid in the solid form ranges from about 1:1 to about 1:2. In one embodiment, the molar ratio is about 1:2 (i.e., dihydrochloride salt).

In one embodiment, Form B is crystalline. In one embodiment, Form B is substantially crystalline. In one embodiment, Form B is moderately crystalline. In one embodiment, Form B is partially crystalline.

In one embodiment, Form B is a solvate of a hydrochloride salt of Compound 3. In one embodiment, Form B is a hydrate of a hydrochloride salt of Compound 3.

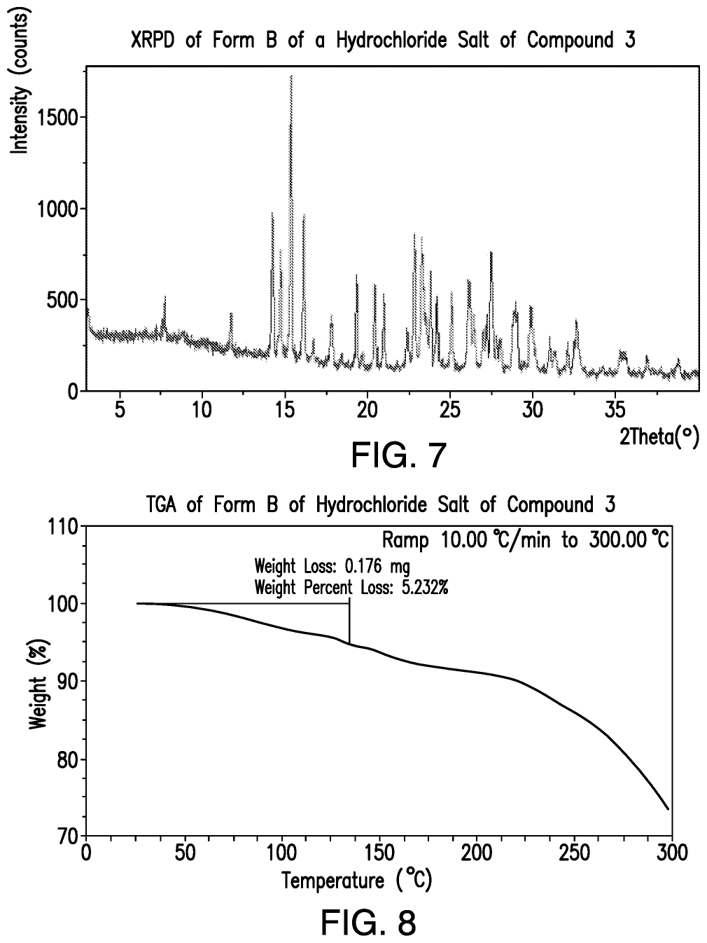

A representative XRPD pattern of the Form B of a hydrochloride salt of Compound 3 is provided in FIG. 7. In one embodiment, provided herein is a solid form comprising a hydrochloride salt of Compound 3, characterized by 1, 2, 3, 4, 5, 6, 7, 8, 9, 10, 11, 12, 13, 14, 15, 16, 17, 18, 19, 20, 21, 22, 23, 24, 25, 26, or all of the XRPD peaks located at approximately the following positions: 7.8, 11.8, 14.3, 14.8, 15.4, 16.2, 16.8, 17.8, 18.5, 19.4, 19.7, 20.5, 21.0, 22.4, 22.8, 23.3, 23.8, 24.2, 25.1, 26.1, 26.4, 27.0, 27.2, 27.5, 27.8, 28.0, and 28.7° 2θ. In one embodiment, the solid form is characterized by 3 of the peaks. In one embodiment, the solid form is characterized by 5 of the peaks. In one embodiment, the solid form is characterized by 7 of the peaks. In one embodiment, the solid form is characterized by 9 of the peaks. In one embodiment, the solid form is characterized by 11 of the peaks. In one embodiment, the solid form is characterized by all of the peaks.

In one embodiment, provided herein is a solid form comprising a hydrochloride salt of Compound 3, characterized by an XRPD pattern comprising peaks at approximately 14.3, 15.4, and 16.2° 2θ. In one embodiment, the XRPD pattern further comprises peaks at approximately 14.8, 17.8, and 19.4° 2θ. In one embodiment, the XRPD pattern further comprises peaks at approximately 7.8 and 21.0° 2θ. In one embodiment, the XRPD pattern comprises peaks at approximately 7.8, 11.8, 14.3, 14.8, 15.4, 16.2, 17.8, 19.4, 20.5, and 21.0° 2θ.

In one embodiment, provided herein is a solid form comprising a hydrochloride salt of Compound 3, characterized by an XRPD pattern that matches the XRPD pattern presented in FIG. 7.

In one embodiment, the XRPD patterns are obtained using Cu Kα radiation.

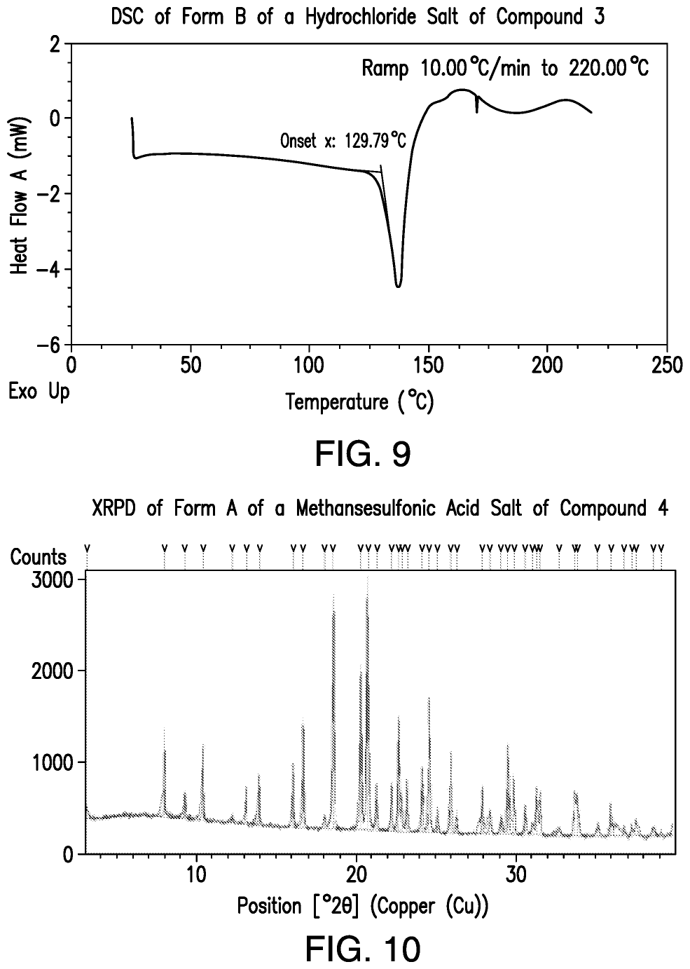

Representative thermal gravimetric analysis (TGA) and differential scanning calorimetry (DSC) thermograms of Form B are provided in FIG. 8 and FIG. 9, respectively. In one embodiment, provided herein is a solid form comprising a hydrochloride salt of Compound 3, which exhibits a weight loss of about 5.2% upon heating from about 25° C. to about 125° C. In one embodiment, provided herein is a solid form comprising a hydrochloride salt of Compound 3, characterized by a TGA thermogram that matches the TGA thermogram presented in FIG. 8.

In one embodiment, provided herein is a solid form comprising a hydrochloride salt of Compound 3, which exhibits, as characterized by DSC, a thermal event (endo) with an onset temperature of about 130° C. In one embodiment, provided herein is a solid form comprising a hydrochloride salt of Compound 3, characterized by a DSC thermogram that matches the DSC thermogram presented in FIG. 9.

In one embodiment, provided herein is a solid form comprising Form B of a hydrochloride salt of Compound 3 and one or more forms of a free base of Compound 3 (e.g., amorphous form and crystalline forms). In one embodiment, provided herein is a solid form comprising Form B of a hydrochloride salt of Compound 3 and amorphous hydrochloride salt of Compound 3. In one embodiment, provided herein is a solid form comprising Form B of a hydrochloride salt Compound 3 and one or more other crystalline forms of a hydrochloride salt of Compound 3. In one embodiment, provided herein is a solid form comprising Form B of a hydrochloride salt of Compound 3 and one or more forms (e.g., amorphous or crystalline) of a salt of Compound 3 provided herein.

### 5.3.4 Form A of Methanesulfonic Acid Salt of Compound 4

In one embodiment, provided herein is a solid form comprising a methanesulfonic acid salt of Compound 4:

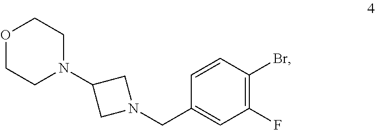 [SMILES: Fc1cc(CN2CC(N3CCOCC3)C2)ccc1Br]

wherein the solid form is Form A (of a methanesulfonic acid salt of Compound 4).

In some embodiments, the molar ratio of Compound 4 to methanesulfonic acid in the solid form ranges from about 1:1 to about 1:2. In one embodiment, the molar ratio is about 1:2 (i.e., bis-methanesulfonic acid salt).

In one embodiment, Form A is crystalline. In one embodiment, Form A is substantially crystalline. In one embodiment, Form A is moderately crystalline. In one embodiment, Form A is partially crystalline.

A representative XRPD pattern of the Form A of a methanesulfonic acid salt of Compound 4 is provided in FIG. 10. In one embodiment, provided herein is a solid form comprising a methanesulfonic acid salt of Compound 4, characterized by 1, 2, 3, 4, 5, 6, 7, 8, 9, 10, 11, 12, 13, 14, 15, 16, 17, 18, 19, 20, 21, 22, 23, 24, 25 or all of the XRPD peaks located at approximately the following positions: 8.0, 9.3, 10.4, 12.2, 13.1, 13.9, 16.0, 16.7, 18.0, 18.6, 20.3, 20.8, 21.3, 22.2, 22.7, 22.9, 23.2, 24.1, 24.6, 25.1, 25.9, 26.3, 27.9, 28.4, 29.1, and 29.5° 2θ. In one embodiment, the solid form is characterized by 3 of the peaks. In one embodiment, the solid form is characterized by 5 of the peaks. In one embodiment, the solid form is characterized by 7 of the peaks. In one embodiment, the solid form is characterized by 9 of the peaks. In one embodiment, the solid form is characterized by 11 of the peaks. In one embodiment, the solid form is characterized by all of the peaks.

In one embodiment, provided herein is a solid form comprising a methanesulfonic acid salt of Compound 4, characterized by an XRPD pattern comprising peaks at approximately 18.6, 20.3, and 20.8° 2θ. In one embodiment, the XRPD pattern further comprises peaks at approximately 16.7 and 22.7° 2θ. In one embodiment, the XRPD pattern further comprises peaks at approximately 8.0 and 24.6° 2θ. In one embodiment, the XRPD pattern comprises peaks at approximately 8.0, 10.4, 13.1, 13.9, 16.0, 16.7, 18.6, 20.3, 20.8, 22.7, and 24.6° 2θ.

In one embodiment, provided herein is a solid form comprising a methanesulfonic acid salt of Compound 4, characterized by an XRPD pattern that matches the XRPD pattern presented in FIG. 10.

In one embodiment, the XRPD patterns are obtained using Cu Kα radiation.

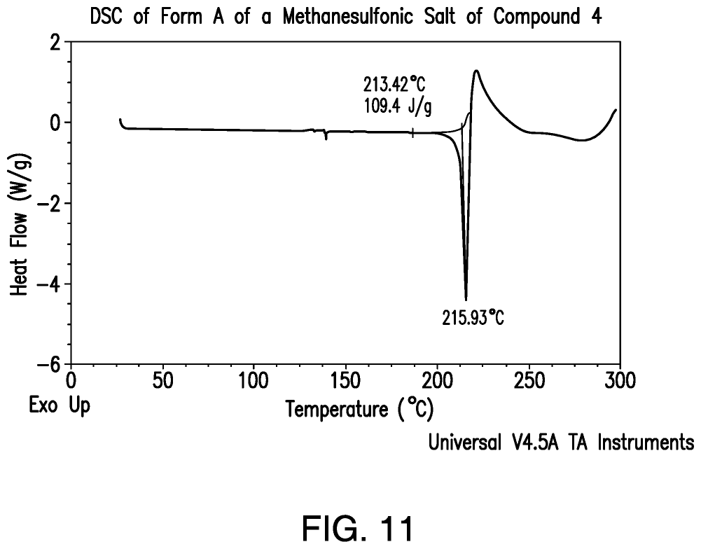

A representative differential scanning calorimetry (DSC) thermogram of Form A of a methanesulfonic acid salt of Compound 4 is provided in FIG. 11. In one embodiment, provided herein is a solid form comprising a methanesulfonic acid salt of Compound 4, which exhibits, as characterized by DSC, a thermal event (endo) with an onset temperature of about 213° C. In one embodiment, the thermal even also has a peak temperature of about 216° C. In one embodiment, provided herein is a solid form comprising a methanesulfonic acid salt of Compound 4, characterized by a DSC thermogram that matches the DSC thermogram presented in FIG. 11.

In one embodiment, Form A of a methanesulfonic acid salt of Compound 4 is prepared by adding methanesulfonic acid to a mixture of Compound 4 in CPME (e.g., at about 50 to about 60° C.), resulting in a slurry, and (ii) slurrying the slurry to provide Form A of a methanesulfonic acid salt of Compound 4. In one embodiment of (ii), the slurry is slurried at about 20° C. for a period of time (e.g., from about 1 hour to about 24 hours, e.g., about 3 to about 4 hours).

In one embodiment, provided herein is a solid form comprising Form A of a methanesulfonic acid salt of Compound 4 and one or more forms of a free base of Compound 4 (e.g., amorphous form and crystalline forms). In one embodiment, provided herein is a solid form comprising Form A of a methanesulfonic acid salt of Compound 4 and amorphous methanesulfonic acid salt of Compound 4. In one embodiment, provided herein is a solid form comprising Form A of a methanesulfonic acid salt Compound 4 and one or more other crystalline forms of a methanesulfonic acid salt of Compound 4. In one embodiment, provided herein is a solid form comprising Form A of a methanesulfonic acid salt of Compound 4 and one or more forms (e.g., amorphous or crystalline) of a salt of Compound 4 provided herein.

All of the combinations of the above embodiments are encompassed by this invention.

### 7. EXAMPLES

As used herein, the symbols and conventions used in these processes, schemes and examples, regardless of whether a particular abbreviation is specifically defined, are consistent with those used in the contemporary scientific literature, for example, the Journal of the American Chemical Society or the Journal of Biological Chemistry. Specifically, but without limitation, the following abbreviations may be used in the examples and throughout the specification: g (grams); mg (milligrams); mL (milliliters); μL (microliters); M (molar); mM (millimolar); μM (micromolar); eq. (equivalent); mmol (millimoles); Hz (Hertz); MHz (megahertz); hr or hrs (hour or hours); min (minutes); and MS (mass spectrometry). Unless otherwise specified, the water content in a compound provided herein is determined by Karl Fisher (KF) method.

For all of the following examples, unless otherwise specified, standard work-up and purification methods known to those skilled in the art can be utilized. Unless otherwise specified, all temperatures are expressed in ° C. (degrees Centigrade). All reactions were conducted at room temperature unless otherwise noted. Synthetic methodologies illustrated herein are intended to exemplify the applicable chemistry through the use of specific examples and are not indicative of the scope of the disclosure.

### Example 1: Synthesis of (S)-2-(2,6-dioxopiperidin-3-yl)-4-((2-fluoro-4-((3-morpholinoazetidin-1-yl)methyl)benzyl)amino)isoindoline-1,3-dione

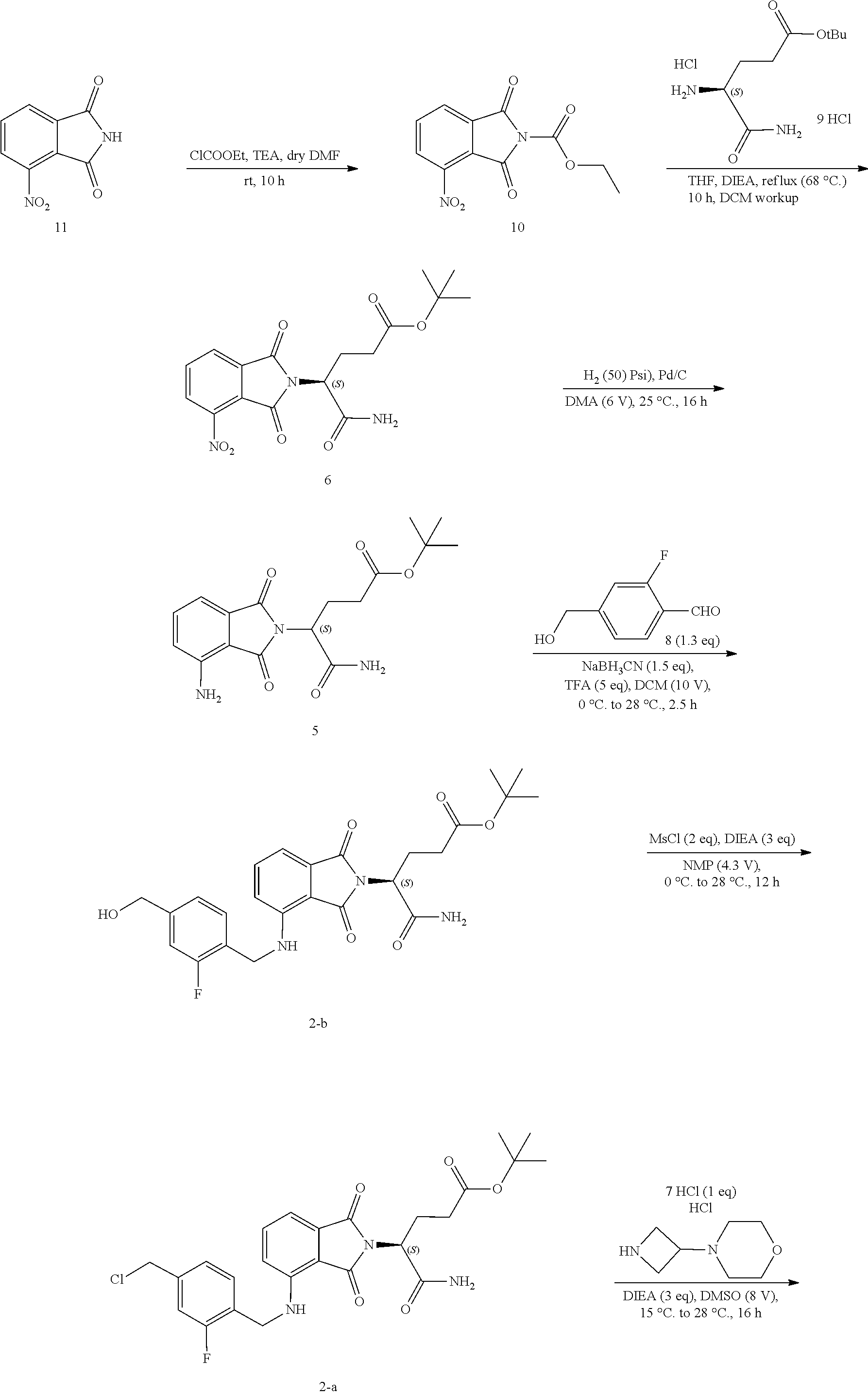

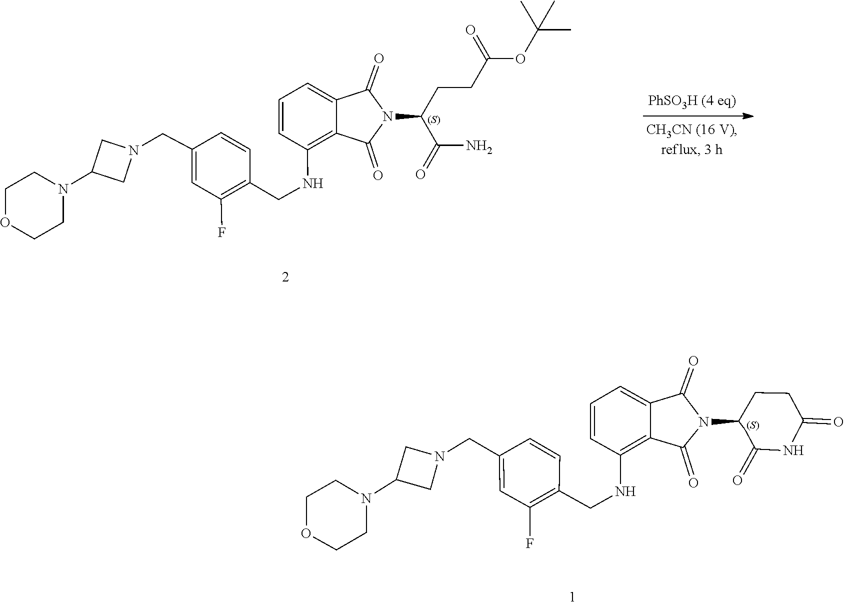

Synthesis of Ethyl 4-nitro-1,3-dioxo-isoindoline-2-carboxylate (Compound 10): To a solution of 4-nitroisoindoline-1,3-dione (Compound 11, 440 g, 2.29 mol) and TEA (262 g, 2.59 mol, 359 mL) in dry DMF (2.2 L) was cooled to 0° C. and ethyl chloroformate (313 g, 2.89 mol, 275 mL) was added dropwise over 5 minutes. The reaction mixture was stirred at 22° C. for 10 hours. The mixture was slowly added to chilled water (10 L) and the resulting suspension stirred for 5 minutes. The suspension was filtered and the filter cake was washed with water (1 L). The solid was dissolved with ethyl acetate (5 L) and the organic phase was washed with aqueous HCl (1 M, 1 L), water (2 L) and brine (2 L). The organic phase was dried over sodium sulfate, filtered and concentrated to give Compound 10 (360 g, 59%) as a white solid. ¹H NMR (400 MHz CDCl₃) δ ppm 8.24 (d, J=7.6 Hz, 1H), 8.19 (d, J=8.4 Hz, 1H), 8.06-8.02 (m, 1H), 4.49 (q, J=7.2 Hz, 2H), 1.44 (t, J=6.8 Hz, 3H).

Synthesis of tert-Butyl (4S)-5-amino-4-(4-nitro-1,3-dioxo-isoindolin-2-yl)-5-oxo-pentanoate (Compound 6): To a solution of Compound 10 (165 g, 625 mmol) and DIEA (113 g, 874 mmol, 153 mL) in dry THF (1700 mL) was added tert-butyl (4S)-4,5-diamino-5-oxo-pentanoate hydrochloride (149 g, 625 mmol) and heated at reflux for 10 hours. The reaction mixture was concentrated under reduced pressure. The resulting residue was diluted with methyl tert-butyl ether (5 L) and stirred at 20° C. for 1 hour. The suspension was filtered and the filter cake was dissolved with DCM (4 L). The organic phase was washed with water (1.5 L×3), brine (1.5 L) and dried over sodium sulfate. The organic phase was filtered and concentrated under reduced pressure to give a light yellow oil. The oil was diluted with hexane/ethyl acetate (10/1, 2 L) and stirred until a light yellow suspension formed. The suspension was filtered and the filter cake was triturated and concentrated in vacuum to give Compound 6 (175 g, 74%) as a light yellow solid. ¹H NMR (400 MHZ CDCl₃) δ ppm 8.12 (d, J=8.0 Hz, 2H), 7.94 (t, J=8.0 Hz, 1H), 6.48 (s, 1H), 5.99 (s, 1H), 4.84-4.80 (m, 1H), 2.49-2.44 (m, 2H), 2.32-2.27 (m, 2H), 1.38 (s, 9H).

Synthesis of tert-Butyl (S)-5-amino-4-(4-amino-1,3-dioxoisoindolin-2-yl)-5-oxopentanoate (Compound 5): To a suspension of Compound 6 (170.0 g, 450.5 mmol, 1.00 eq) in DMA (1.00 L) was added palladium on carbon (50.0 g, 10% purity) under nitrogen. The suspension was degassed under vacuum and purged with hydrogen gas several times. The mixture was stirred under hydrogen gas (50 psi) at 25° C. for 16 hours. The mixture was filtered and the filtrate was poured into cooled water (3.0 L). The mixture was stirred at 10° C. for 1 hour and filtered. The filter cake was washed with water (700 mL) and dissolved in DCM (1.00 L). The organic phase was dried over sodium sulfate, filtered and concentrated under reduced pressure to give Compound 5 (107 g, 68%) as a green solid. ¹H NMR (400 MHZ DMSO-d₆) δ ppm 7.52 (s, 1H), 7.43 (dd, J=8.4, 7.2 Hz, 1H), 7.13 (s, 1H), 6.95-6.99 (m, 2H), 6.42 (s, 2H), 5.75 (s, 1H), 4.47-4.51 (m, 1H), 2.32-2.33 (m, 1H), 2.14-2.20 (m, 3H), 1.32 (s, 9H); HPLC purity, 100.0%; SFC purity, 100.0% ee.

Synthesis of 2-fluoro-4-(hydroxymethyl)benzaldehyde (Compound 8): To a solution of 4-(((tert-butyldimethylsilyl)oxy)methyl)-2-fluorobenzaldehyde (370.0 g, 1.38 mol, 1.00 eq) in THF (1.85 L) was added a solution of p-toluenesulfonic acid monohydrate (78.7 g, 413.6 mmol, 0.30 eq) in water (1.85 L) drop-wise at 10° C. The mixture was stirred at 27° C. for 16 hours. TEA (80 mL) was added drop-wise and stirred for 10 minutes. The organic phase was separated and the aqueous phase was extracted with ethyl acetate (600 mL×4). The combined organic phase was washed with brine (1.50 L), dried over anhydrous sodium sulfate, filtered and concentrated under reduced pressure. The residue was purified by silica gel column chromatography to give Compound 8 (137.5 g, 76%) as a yellow oil. ¹H NMR (400 MHZ CDCl₃) δ ppm 10.34 (s, 1H), 7.86 (dd, J=8.0, 7.2 Hz, 1H), 7.25 (s, 1H), 7.22 (d, J=4.4 Hz, 1H), 4.79 (d, J=6.0 Hz, 2H), 1.91 (t, J=6.0 Hz, 1H).

Synthesis of tert-Butyl (S)-5-amino-4-(4-((2-fluoro-4-(hydroxymethyl)benzyl)amino)-1,3-dioxoisoindolin-2-yl)-5-oxopentanoate (Compound 2-b): To a solution of Compound 5 (100.0 g, 287.9 mmol, 1.00 eq) and Compound 8 (57.7 g, 374.3 mmol, 1.30 eq) in dry DCM (1.00 L) was added TFA (164.1 g, 1.44 mol, 5.00 eq) at 0° C. The reaction mixture was stirred at 28° C. for 2 hours. To the solution was added sodium cyanoborohydride (27.1 g, 431.8 mmol, 1.50 eq) at 0° C. The mixture was stirred at 28° C. for 30 minutes. The reaction mixture was quenched by addition of MeOH (600 mL) and concentrated under reduced pressure. The residue was purified by silica gel column chromatography to give Compound 2-b (110.0 g, 74.0%) as a yellow solid. ¹H NMR (400 MHZ, DMSO-d₆) δ ppm 7.56 (s, 1H), 7.50 (dd, J=8.4, 7.2 Hz, 1H), 7.34 (t, J=8.0 Hz, 1H), 7.02-7.18 (m, 4H), 6.94-7.01 (m, 2H), 4.57 (d, J=6.0 Hz, 2H), 4.47-4.53 (m, 3H), 2.31-2.35 (m, 1H), 2.15-2.22 (m, 3H), 1.31 (s, 9H); HPLC purity, 94.0%; SFC purity, 100.0% ee.

Synthesis of tert-butyl (S)-5-amino-4-(4-((4-(chloromethyl)-2-fluorobenzyl)amino)-1,3-dioxoisoindolin-2-yl)-5-oxopentanoate (Compound 2-a): To a solution of Compound 2-b (100.0 g, 206.0 mmol, 1.00 eq) in NMP (430.0 mL) was added DIEA (79.9 g, 617.9 mmol, 3.00 eq) and MsCl (47.2 g, 411.9 mmol, 2.00 eq) at 0° C. The ice bath was removed, and the reaction was stirred at 28° C. for 10 hours. The reaction was poured into cooled water (<10° C., 2.0 L) and stirred for 10 minutes. The mixture was extracted with methyl tert-butyl ether (750 mL×3). The combined organic layer was washed with brine (1.25 L), dried over sodium sulfate, filtered and concentrated under reduced pressure. The residue was purified by silica gel column chromatography to give Compound 2-a (86.0 g, 81.2%) as a yellow solid. ¹H NMR (400 MHz DMSO-d₆) δ ppm 7.55 (s, 1H), 7.50 (dd, J=8.4, 7.2 Hz, 1H), 7.38 (t, J=8.0 Hz, 1H), 7.31 (dd, J=10.8, 1.6 Hz, 1H), 7.23 (dd, J=8.0, 1.6 Hz, 1H), 7.16 (s, 1H), 7.11 (t, J=6.4 Hz, 1H), 7.00 (d, J=7.2 Hz, 1H), 6.95 (d, J=8.4 Hz, 1H), 4.74 (s, 2H), 4.61 (d, J=6.4 Hz, 2H), 4.49-4.53 (m, 1H), 2.29-2.38 (m, 1H), 2.16-2.25 (m, 3H), 1.30 (s, 9H); HPLC purity, 98.0%; SFC purity, 100.0% ee.

Synthesis of tert-butyl (S)-5-amino-4-(4-((2-fluoro-4-((3-morpholinoazetidin-1-yl)methyl)benzyl)amino)-1,3-dioxoisoindolin-2-yl)-5-oxopentanoate (Compound 2): To a solution of 4-(azetidin-3-yl)morpholine hydrochloride (Compound 7 HCl, 30.5 g, 170.7 mmol, 1.00 eq) and DIEA (66.2 g, 512.0 mmol, 3.00 eq) in DMSO (350.0 mL) was added a solution of Compound 2-a (86 g, 170.65 mmol, 1.00 eq) in DMSO (350.0 mL) drop-wise at 15° C. The reaction mixture was stirred at 28° C. for 16 hours. The reaction mixture was poured into cold half saturated brine (<10° C., 2.5 L) and extracted with ethyl acetate (1.50 L, 1.00 L, 800.0 mL). The combined organic phase was washed with saturated brine (1.50 L), dried over sodium sulfate, filtered, and concentrated under reduced pressure. The residue was purified by silica gel column chromatography to give Compound 2 (68.3 g, 65.7%) as a yellow solid. ¹H NMR (400 MHz DMSO-d₆) δ ppm 7.55 (s, 1H), 7.50 (dd, J=8.4, 7.2 Hz, 1H), 7.31 (t, J=8.0 Hz, 1H), 7.16 (s, 1H), 6.94-7.10 (m, 5H), 4.56 (d, J=6.4 Hz, 2H), 4.49-4.52 (m, 1H), 3.54-3.55 (m, 6H) 3.31-3.32 (m, 3H), 2.81-2.88 (m, 3H), 2.29-2.38 (m, 1H), 2.15-2.25 (m, 7H), 1.30 (s, 9H); HPLC purity, 100.0%; SFC purity, 100.0% ee.

Synthesis of (S)-2-(2,6-dioxopiperidin-3-yl)-4-((2-fluoro-4-((3-morpholinoazetidin-1-yl)methyl)benzyl)amino)isoindoline-1,3-dione (Compound 1): A solution of Compound 2 (30.0 g, 49.2 mmol, 1.00 eq) and benzenesulfonic acid (31.1 g, 196.8 mmol, 4.00 eq) in acetonitrile (480.0 mL) was stirred at reflux for 3 hours. The reaction was cooled to 20° C., poured into cold brine:saturated sodium bicarbonate solution (1:1, <10° C., 2.0 L) and extracted with ethyl acetate (1.0 L). The organic phase was washed with cold brine:saturated sodium bicarbonate solution (1:1, <10° C., 1.00 L) once more. The combined aqueous phase was extracted with ethyl acetate (500.0 mL×2). The combined organic phase was washed with cold brine (<10° C., 1.0 L), dried over sodium sulfate, filtered and concentrated under reduced pressure. The crude product was purified by silica gel column chromatography to give Compound 1 (17.5 g, 66.0%) as a yellow solid. ¹H NMR (400 MHZ, DMSO-d₆) δ ppm 11.10 (s, 1H), 7.54 (t, J=8.0 Hz, 1H), 7.30 (t, J=8.0 Hz, 1H), 7.04-7.10 (m, 4H), 7.00 (d, J=8.4 Hz, 1H), 5.07 (dd, J=12.8, 5.2 Hz, 1H), 4.58 (d, J=6.4 Hz, 2H), 3.53-3.55 (m, 6H), 3.30-3.32 (m, 2H), 2.81-2.89 (m, 4H), 2.54-2.61 (m, 2H), 2.20 (m, 4H) 2.03-2.06 (m, 1H); HPLC purity, 100.0%; SFC purity, 97.2% ee; LCMS (ESI) m/z 536.1 [M+H]⁺.

### Example 2: Synthesis of (S)-2-(2,6-dioxopiperidin-3-yl)-4-((2-fluoro-4-((3-morpholinoazetidin-1-yl)methyl)benzyl)amino)isoindoline-1,3-dione

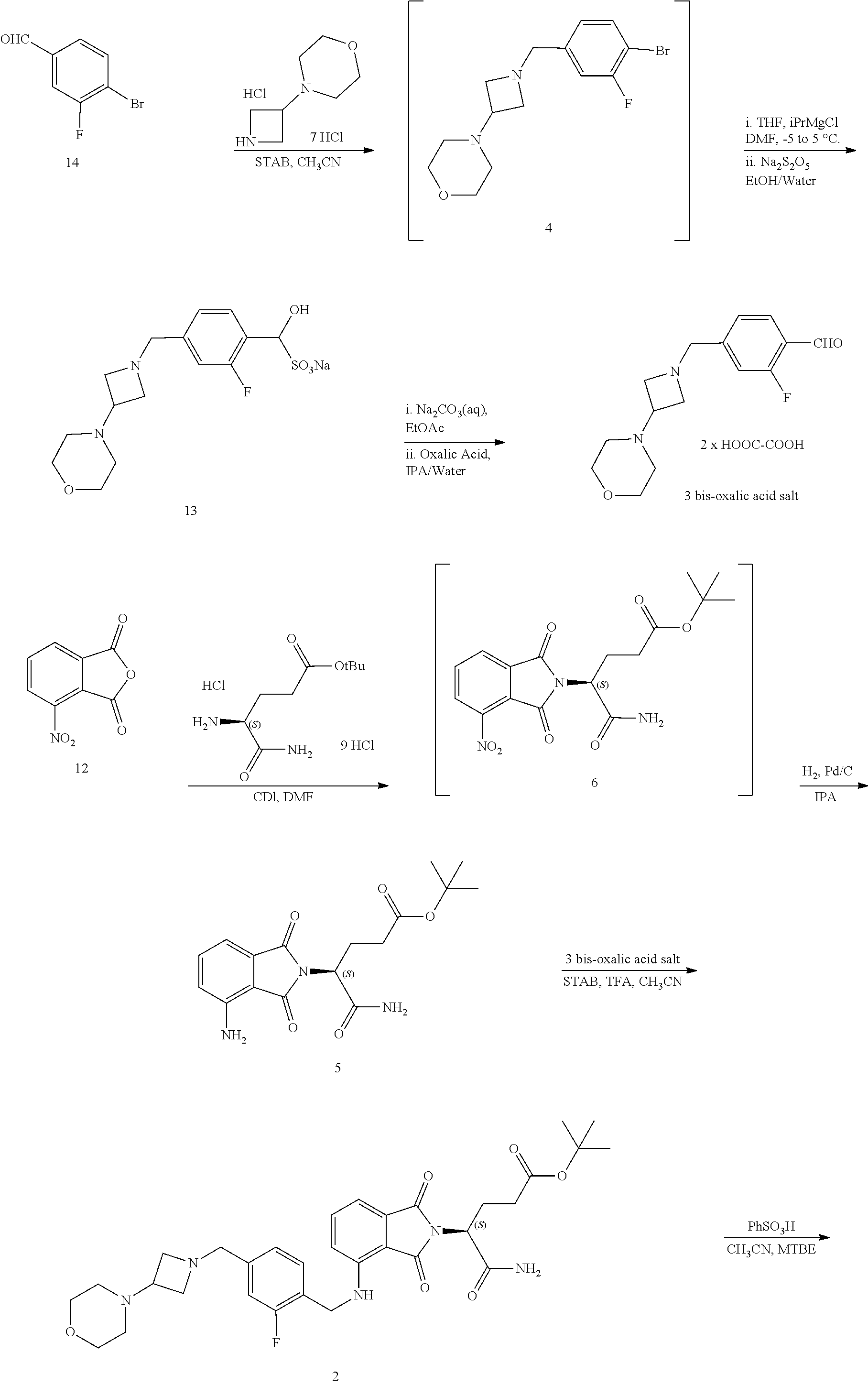

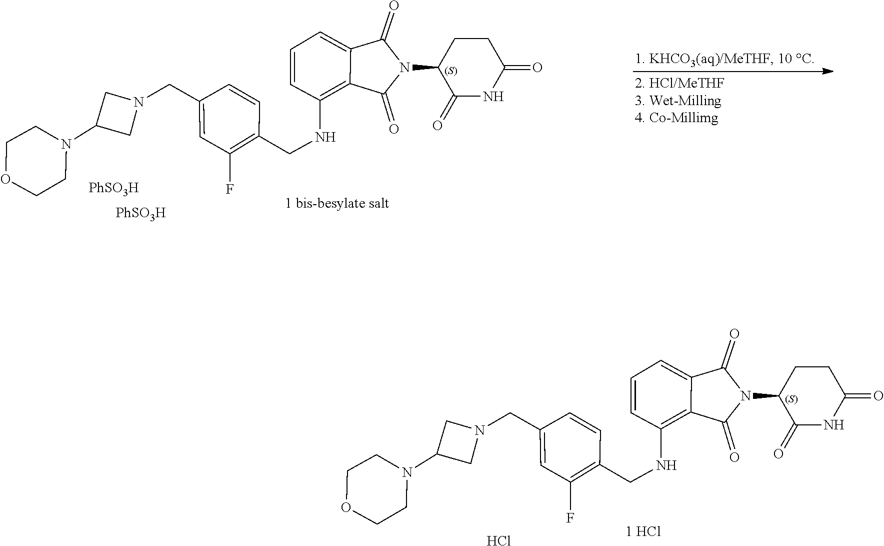

Synthesis of tert-butyl (S)-5-amino-4-(4-nitro-1,3-dioxoisoindolin-2-yl)-5-oxopentanoate (Compound 6): Ethyl acetate (245 mL, 5 V), 3-nitrophthalic anhydride (49.1 g, 0.25 mol, 1 eq), and tert-butyl (S)-4,5-diamino-5-oxopentanoate hydrochloride (59.2 g, 0.25 mol, 1 eq) were charged into a reactor and cooled to 15-20° C. A premade solution of CDI (66.7 g, 0.41 mol, 1.5 eq) in DMF (245 mL, 5 V) was charged and the mixture was stirred at 20-25° C. for 1 hour. The reaction was quenched with 15% (wt/wt) aqueous citric acid solution (10 V). EtOAc (5 V) was added, the mixture was agitated and the phases split and separated. The aqueous layer was extracted with EtOAc (5 V) and the combined organic layers were washed twice with a 5% (wt/wt) aqueous citric acid solution (5 V each wash). The organic layer was distilled at reduced pressure to 5 V and further continuously distilled at reduced pressure with the addition of iPrOH (10 V), maintaining a constant volume at 5 V. The final distillate was diluted to 13 V with iPrOH and used in the next step without further manipulation. 91% solution yield.

Synthesis of tert-butyl (S)-5-amino-4-(4-amino-1,3-dioxoisoindolin-2-yl)-5-oxopentanoate (Compound 5): The solution of Compound 6 in iPrOH was charged to a hydrogenation reactor. 10% palladium on carbon (50% wet, 4.65 g 5 wt %) was charged. The reaction mixture was stirred under 50-60 psi H₂ at 40-50° C. for 16 hrs. The reaction mixture was filtered and the filter cake was washed three times with iPrOH (1 V each wash). The solution was distilled at reduced pressure to 5 V, cooled to ambient temperature and seeded (1 wt %). Water (20 V) was charged at 20-25° C. The resultant slurry was cooled to 3-8° C. for 4-8 hrs. The solids were collected by filtration and washed three times with cold water (1.5 V each wash). The solids were dried at 35-45° C. under reduced pressure to give Compound 5 in 87% yield. ¹H NMR (500 MHz DMSO-d₆) δ (ppm): 7.52 (s, 1H), 7.43 (dd, J=8.4, 7.0 Hz, 1H), 7.13 (s, 1H), 6.97 (ddd, J=10.9, 7.7, 0.61 Hz, 2H), 6.43 (s, 2H), 4.49 (m, 1H), 2.33 (m, 1H), 2.17 (m, 3H), 1.32 (s, 9H); HPLC purity, 99.2%; Chiral purity, 99.9% ee; LCMS (ESI) m/z 348.2, [M+H]⁺, 292.2 [M-t-Bu+H]⁺. Residual IPA: 0.7 mol % by ¹H NMR.

Synthesis of 4-(1-(4-bromo-3-fluorobenzyl)azetidin-3-yl)morpholine (Compound 4): A mixture of 4-bromo-3-flurobenzaldehyde (Compound 14, 82 g, 396 mmol) and 4-(azetidin-3-yl)-morpholine hydrochloride (Compound 7 HCl, 72 g, 396 mmol) in acetonitrile (820 ml) was agitated at 25±5° C. for at least 3 hours. The mixture was cooled to 10±5° C. and sodium triacetoxyborohydride (130 g, 594 mmol) was added in four portions while maintaining the temperature of the mixture below 30° C. The temperature of the mixture was adjusted to 25±5° C. and stirred for at least 30 min until reaction completion. The mixture was transferred to a precooled (10-15° C.) solution of aqueous citric acid (152 g in 400 ml water, 792 mmol) while maintaining the temperature below 30° C. Once the quenching process was complete, the mixture was concentrated to ˜560 ml (7 volumes) while keeping the temperature at or below 45° C. The mixture was then washed with toluene (320 ml). To the aqueous phase was added THF and the pH was adjusted to above 12 with aqueous NaOH solution (320 ml, 10 N). The phases were separated, and the aqueous phase was removed. The organic phase was washed with brine and subsequently concentrated with addition of THF (˜3 L) until KF≤0.10%. The mixture was filtered to remove any inorganics and the product Compound 4 was isolated as a solution in THF with 95% yield.

Synthesis of sodium (2-fluoro-4-((3-morpholinoazetidin-1-yl)methyl)phenyl) (hydroxy)methanesulfonate (Compound 13): A solution of Compound 4 (520 g, 1.58 mol) in THF (380 ml) was cooled to −15±5° C. A solution of iPrMgCl·LiCl (1.3 M, 1823 ml, 2.37 mol) in THF was added over the course of at least 1 hour while maintaining the temperature below −10° C. After addition was complete, the temperature of the reaction mixture was adjusted to 0±5° C. and stirred for at least 1 hour. Once magnesiation was complete, the mixture was cooled to −15±5° C. (target −15° C. to −20° C.) and a solution of DMF (245 ml g, 3.16 mol) in THF (260 ml) was added slowly over the course of at least 1 hour while maintaining the temperature below −10° C. The temperature of the mixture was then adjusted to −15±5° C. and agitated for at least 4 hours.

Upon reaction completion, the reaction mixture was charged into an aqueous 3 N HCl solution (2600 ml) over the course of at least 1 hour while maintaining the temperature below −5° C. The temperature of the mixture was then adjusted to 5±5° C. and agitation was stopped, letting the mixture settle for at least 15 minutes. The layers were separated. The lower aqueous layer containing the product was washed with 2-MeTHF (2600 ml). The aqueous layer was then charged with 2-MeTHF (2600 ml) and the temperature of the batch was adjusted to −10±5° C. To the cooled mixture, an aqueous 5 N NaOH (728 ml, 3.64 mol) solution was added while maintaining the temperature below −5° C. until the pH of the mixture was between 10 and 11. The temperature of the mixture was adjusted to 5±5° C. and agitated for at least 15 minutes. The agitation of the mixture was stopped and the mixture allowed to settle for at least 15 minutes. The layers were separated, and the lower aqueous layer was back extracted two times with 2-MeTHF (2600 ml). The combined organic layer was washed with water (1040 mL) and the organic solution was evaporated to dryness, affording 372 g of crude Compound 3 freebase as an oil (yield 85%). ¹H NMR (DMSO-d₆) δ (ppm): 10.18 (s, 1H), 7.78 (t, J=7.7 Hz, 1H), 7.23-7.35 (m, 2H), 3.66 (s, 2H), 3.51-3.60 (m, 4H), 3.26-3.47 (m, 2H), 2.72-2.97 (m, 3H), 2.12-2.32 (m, 4H).

The crude Compound 3 freebase (4.3 kg) was adsorbed onto silica gel (8.6 kg) with 100% DCM, loaded onto a 60 L column containing 12.9 kg silica gel (packed with 100% DCM), and eluted with DCM (86 L), followed successively by 1% MeOH/DCM (40 L), 3% MeOH/DCM (80 L) and 10% MeOH/DCM (40 L). The fractions were collected and concentrated at or below 38° C. to give Compound 3 as a purified oil (3.345 kg, yield 66%).

A portion of Compound 3 (1.0 kg, 3.59 mol) was dissolved in ethanol (16.0 L, 16 vol) at 20±5° C. and the mixture heated to 40° C. A solution of Na₂S₂O₅ (622.0 g, 3.27 mol; 0.91 eq) in water (2 L, 2 vol) was prepared at 20±5° C. and added to the freebase solution at 40° C. to obtain an off-white suspension. The batch was agitated and maintained at 40° C. for 2 hrs, then cooled to 20±5° C. and agitated for 1 to 2 hrs. The batch was filtered and washed with ethanol (2×2.0 L, 2×2 vol) to obtain an off-white solid. The wet cake was dried under vacuum at 40° C. for 18 hrs to afford about 1.88 kg of Compound 13.

Synthesis of 2-fluoro-4-((3-morpholinoazetidin-1-yl)methyl)benzaldehyde (Compound 3): Compound 13 (1.88 kg) was dissolved in ethyl acetate (15.0 L) at 20±5° C. A 2 M Na₂CO₃ solution (total 15.0 L used) was added to adjust the pH to 10.0. The batch was agitated for 1 to 1.5 hrs at 20±5° C. After the reaction was complete, the phases were separated and the organic layer was washed with brine (2.0 L). The organic layer was concentrated to dryness at 35-38° C. to afford 852.0 g of Compound 3 as a colorless oil (yield 81%).

Synthesis of 2-fluoro-4-((3-morpholinoazetidin-1-yl)methyl)benzaldehyde bis-oxalic acid salt (Compound 3 bis-oxalic acid salt): A portion of the Compound 3 oil (187 g, 0.67 mol) was dissolved in isopropanol (1125 ml) and water (375 ml). A first portion (˜30%) of this freebase mixture (480 ml) was slowly added over the course of at least 30 minutes to a solution of oxalic acid (125 g, 1.38 mol) in IPA (1125 ml)/water (375 ml) at 60±5° C. A second portion (˜20%) of the freebase mixture (320 ml) was slowly added over the course of at least 30 minutes to the reaction mixture at 60±5° C. The reaction mixture was agitated at 60±5° C. for at least 90 minutes. A third portion (˜25%) of the freebase mixture (˜400 ml) was slowly added over the course of at least 30 minutes to the reaction mixture at 60±5° C. and the reaction mixture was agitated at 60±5° C. for at least 90 minutes. The remaining freebase solution (400 ml) was slowly added over the course of at least 30 minutes to the reaction mixture at 60±5° C. and the reaction mixture was agitated at 60±5° C. for at least 90 minutes. The temperature of the mixture was adjusted to 20±5° C. (target 20)° C. over the course of at least 1 hour and the mixture was agitated for at least 16 hours at 20±5° C. and then filtered. The cake was washed three times with IPA (2×375 ml) and dried in the drying oven at ≤40° C. with a slow bleed of nitrogen to afford 261 g of Compound 3 bis-oxalic acid salt (yield 85%). ¹H NMR (DMSO-d₆) δ (ppm): 10.21 (s, 1H), 7.87 (t, J=7.6 Hz, 1H), 7.42-7.56 (m, 2H), 4.31 (s, 2H), 3.89-4.03 (m, 2H), 3.75-3.89 (m, 2H), 3.60 (br t, J=4.3 Hz, 4H), 3.26 (br t, J=6.9 Hz, 1H), 2.37 (br s, 4H).

Synthesis of tert-butyl (S)-5-amino-4-(4-((2-fluoro-4-((3-morpholinoazetidin-1-yl)methyl)benzyl)amino)-1,3-dioxoisoindolin-2-yl)-5-oxopentanoate (Compound 2): Acetonitrile (6.8 L, 8.0× Vol) was added to a 30 L jacketed cylindrical reactor. Compound 5 (0.845 kg, 1.00× Wt) and Compound 3 bis-oxalic acid salt (1.35 kg, 1.60× Wt) were charged into the reactor, followed by additional acetonitrile (5.9 L, 7.0× Vol). The contents of the reactor were equilibrated with agitation to 20±5° C. Trifluoroacetic acid (0.19 L, 0.22× Vol) was added dropwise, maintaining the batch temperature at 20±5° C. The reaction mixture was stirred at 20±5° C. for no less than 5 minutes and then sodium triacetoxyborohydride (0.13 kg, 015× Wt) was added as a solid, maintaining the batch temperature at 20±5° C. The process of adding trifluoroacetic acid and then sodium triacetoxyborohydride was repeated an additional 5 times. After the last addition, the reaction mixture was sampled to determine the reaction progress. The reaction was held at 20±5° C. overnight. The reaction mixture was then quenched with water (3.4 L, 4.0× Vol), maintaining the batch temperature at 20±5° C. The mixture was then stirred at 20±5° C. for no less than 30 minutes and the resultant slurry filtered through a 3 L sintered glass filter, directing the filtrates to clean containers. The reactor was rinsed with acetonitrile (0.4 L, 0.5× Vol) and the rinse passed through the contents of the 3 L sintered glass filter, directing the filtrate to the containers containing the main batch. The contents of the containers were concentrated to ˜5× Vol under reduced pressure at a bath temperature of no more than 30° C. The residue was transferred to a clean reactor, was rinsing with 2-MeTHF (2.5 L, 3.0× Vol) to complete the transfer. Additional 2-MeTHF (10.1 L, 12.0× Vol) was added to the reactor, followed by water (3.4 L, 4.0× Vol). The mixture was agitated for no less than 15 minutes at 20±5° C., then allowed to settle for no less than 10 minutes at 20 #5° C. before transferring the bottom aqueous layer to new containers. An aqueous sodium bicarbonate solution (5.3 L, 6.3× Vol, 9% wt/wt) was added to the reactor with agitation over 30 minutes, maintaining batch temperature no more than 25° C. The mixture was agitated for no more than 15 minutes at 20±5° C., then allowed to settle for no less than 10 minutes at 20±5° C. before the bottom aqueous layer was transferred to new containers. The aqueous sodium bicarbonate wash was repeated an additional 2 times to reach a pH of about 6.6 for the spent aqueous layer. A saturated aqueous solution of NaCl (0.85 L, 1.0× Vol) was then added to reactor with agitation. The mixture was agitated for no less than 15 minutes at 20±5° C., then allowed to settle for no less than 10 minutes before the bottom aqueous layer was transferred to new containers. The remaining organics were concentrated under reduced pressure to a batch volume of ˜5× Vol at a bath temperature of about 40° C. Acetonitrile (5.1 L, 6.0× Vol) was added to the residual volume and the resulting solution concentrated to a batch volume of ˜5×Vol under reduced pressure at bath temperature of about 40° C. The process of adding acetonitrile and concentrating under vacuum was repeated two more times to reach the distillation endpoint with a water content of about 1%. The acetonitrile solution was transferred to a clean container along with two 1.7 L (2.0× Vol) rinses and held at 5° C. overnight. The acetonitrile solution was then filtered through a 3 L sintered glass filter, followed by a 1.7 L (2.0× Vol) acetonitrile rinse, directing the filtrates to a clean container. The filtrate was transferred to a clean reactor and the container rinsed twice with 1.7 L (2.0× Vol) of acetonitrile to complete the transfer. Enough acetonitrile (roughly 0.6 L) was added to adjust the total volume in the reactor to about 14 L. A solution assay of the contents of the reactor was obtained to calculate the amount of Compound 2 present for use in the next step (result=1.3 kg=1.00× Wt for remainder of process).

Synthesis of (S)-2-(2,6-dioxopiperidin-3-yl)-4-((2-fluoro-4-((3-morpholinoazetidin-1-yl)methyl)benzyl)amino)isoindoline-1,3-dione bis-besylate salt (Compound 1 bis-besylate salt): The solution of Compound 2 in acetonitrile from the previous step was diluted with acetonitrile (roughly 2 L) such that the total volume in the reactor was about 16 L. The solution was cooled with agitation to 10±5° C. and held within that range for 96 hours. Benzenesulfonic acid (1.86 kg, 1.43× Wt) was added while sparging the reaction mixture with nitrogen gas and maintaining the batch temperature at 10±10° C. The temperature of the reactor was then adjusted to 20±5° C. and the mixture stirred at that temperature for 60 minutes. The total volume of reaction mixture was adjusted back to 16 L to account for solvent lost during sparging by the addition of acetonitrile (roughly 0.4 L). The reaction mixture was then heated to 55±5° C. over the course of about 30 minutes and held in that range for 15 to 16 hours for reaction completion. The mixture was then cooled to 50±5° C. and MTBE (3.9 L, 3.0× Vol) was added, maintaining the batch temperature at 50±5° C. The mixture was allowed to stir at 50±5° C. for about 1.5 hours to establish a self-seeded slurry. Additional MTBE (3.9 L, 3.0× Vol) was added to the reactor over the course of about 1.75 hours at 50±5° C. The slurry was cooled to 20±5° C. over the course of about 1.75 hours and held in that temperature range overnight. The slurry was filtered using a Buchner funnel. The reactor was rinsed twice with MTBE (3.9 L each, 3.0× Vol) and the rinse was used to wash the solids in the Buchner funnel. The solids were dried on drying trays for about 23 hours at 40° C. under reduced pressure (15-150 mbar), yielding 1.62 kg (77.9%) of Compound 1 bis-besylate salt.

Synthesis of (S)-2-(2,6-dioxopiperidin-3-yl)-4-((2-fluoro-4-((3-morpholinoazetidin-1-yl)methyl)benzyl)amino)isoindoline-1,3-dione hydrochloride salt (Compound 1 HCl): A suspension of Compound 1 bis-besylate salt (120 g, 1 equiv.) in 2-MeTHF (25 L/kg) was added to a reactor and agitated at 10° C. A solution of KHCO₃ (32.5 g, 2.4 equiv) in water (1.8 L, 6 L/kg) was added to the slurry over the course of 40 minutes. The mixture was stirred for an additional 30 minutes. The batch was then allowed to settle, at which point the aqueous (bottom) layer was separated and discarded. An aqueous solution of NaCl (5%, 5 L/kg, 575 ml) was added to the organic layer and the mixture was agitated for 10 minutes, after which point the temperature was raised to 20° C. The batch was allowed to settle, at which point the aqueous (bottom) layer was discarded. The brine was repeated a second time. Additional 2-MeTHF (500 ml) was added to dilute the organic layer, resulting in a concentration of about 20 mg product per ml. A solution of HCl (total 0.98 eq.) in 2-MeTHF was prepared and a portion (20% of total, corresponding to ˜0.2 eq.) then added to the reaction mixture over the course of about 10 min. Seeds of Compound 1 hydrochloride (˜5% wt) were added, but did not dissolve. The batch was held under vigorous agitation for one hour. To the slurry, the remaining portion of the HCl solution (˜0.78 eq.) was added over the course of 3 hours at a constant rate. Vigorous agitation was maintained. After addition was complete, the batch was held for one hour, after which the batch was filtered, washed three times with 3 L/kg of 2-MeTHF. The filter cake was placed in a vacuum oven at 22° C. for 12 hours, at which point the temperature was raised to 40° C. Dry cake of Compound 1 hydrochloride (58 g, 75% yield) was obtained and packaged. Achiral HPLC purity: 98.91%; chiral HPLC purity: 99.68%.

### Example 3: Additional Information for Preparing Compound 1 Hydrochloride Salt from Compound 1 Bis-Besylate Salt

The free base of Compound 1 was sensitive to aqueous base and racemization was observed. The rate is time and temperature sensitive (Table 1). The isolation of the crystalline bis-besylate salt of Compound 1 avoids the need for a pH swing. Also, the crystalline freebase has poor morphology which makes filtration slow, increasing the risk for racemization. Racemization was observed during filtration as well. Chiral purity data in Table 2 highlights the advantage of isolating the more stable bis-besylate salt compared to the crystalline freebase.

TABLE 1
Chiral Stability of Compound 1 Free
Base in Aqueous at Various pH

| Entry | pH | Time (h) | Solubility (mg/mL) | Chiral Purity (%) |
|---|---|---|---|---|
| 1 | 5.3 | 3 | 4.5 | 94.1 |
| 2 | 6.7 | 4 | 2.4 | 89.1 |
| 3 | 7.6 | 4 | 0.4 | 83.2 |

TABLE 2

| | | | Chemical | Chiral |
|---|---|---|---|---|
| Entry | Process | Yield | Purity | Purity |
| 1 | Isolation of crystalline freebase of Compound 1 after pH | 98.6% | 96% | 97.5% |
| | adjustment with aqueous K₂CO₃ followed by crystallization. | | | |
| 2 | Direct Isolation of the bis-besylate salt of Compound 1 | 78%* | 98.2 | 99.6% |
| | according to process in Example 2 | | | |
| 3 | Direct Isolation of the bis-besylate salt of Compound 1 | 79%* | 99.2 | 99.5% |
| | according to process in Example 2 | | | |

*Over-all yield from Compound 5 (over 2 Steps).

No upgrade was observed in terms of both achiral and chiral purity from the crystallization of Compound 1 hydrochloride salt from isolated Compound 1 freebase. Isolation of the freebase resulted in material with poor crystallinity, which resulted in slow filtration and ultimately erosion in chiral purity over time. The HPLC purity on the isolated freebase was 95.8% and the chiral purity was 97.5% (Table 2). On the other hand, the process in Example 2 involving the freebasing of bis-besylate salt followed by crystallization of the hydrochloride salt from the solution results in significant upgrade (Table 3). Without being limited by a particular theory, it is the biphasic nature of this salt break that is key to the purity upgrade.

TABLE 3

| | | Input | Chemical | Chrial |
|---|---|---|---|---|
| Entry | Process | Purity | Purity | Purity |
| 1 | process involving freebasing bis-besylate salt of Compound | 98.2 | 99.8% | 99.6% |
| | 1 followed by HCl formation from the organic solution. | | | |
| 2 | process involving freebasing bis-besylate salt of Compound | 99.2 | 99.8% | 99.5% |
| | 1 followed by HCl formation from the organic solution. | | | |

### Example 4: Synthesis of (S)-2-(2,6-dioxopiperidin-3-yl)-4-((2-fluoro-4-((3-morpholinoazetidin-1-yl)methyl)benzyl)amino)isoindoline-1,3-dione

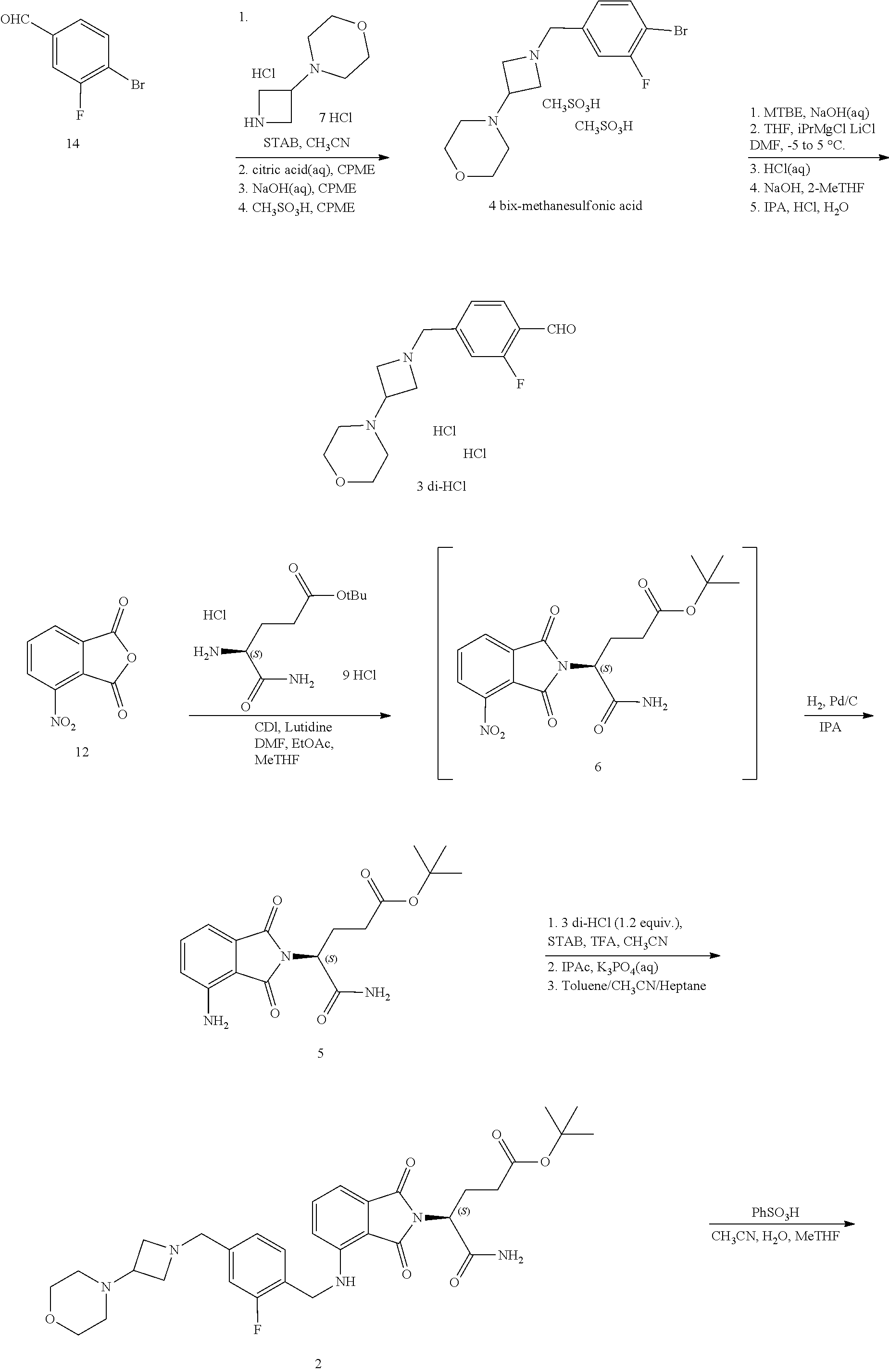

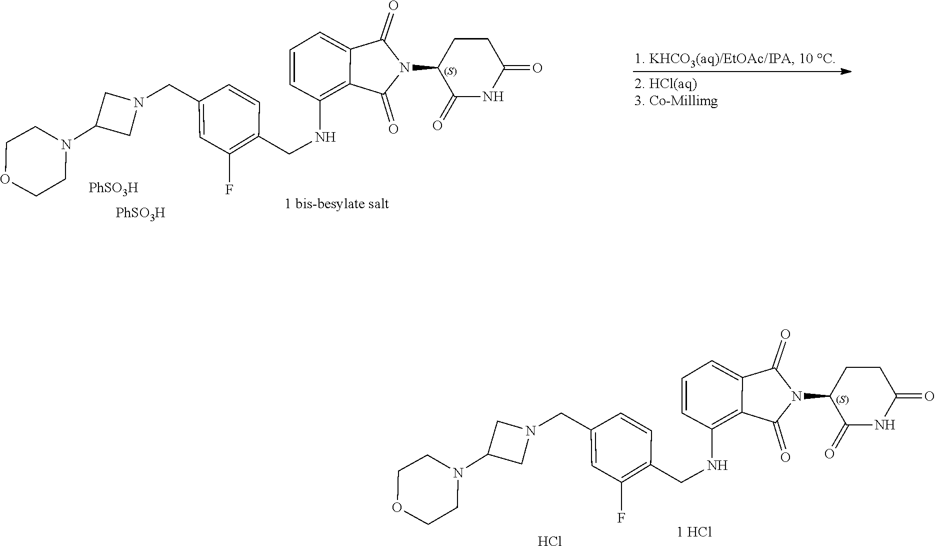

Synthesis of tert-butyl (S)-5-amino-4-(4-nitro-1,3-dioxoisoindolin-2-yl)-5-oxopentanoate (Compound 6): To a solution of 3-nitrophthalic anhydride (Compound 12, 35.15 g, 176.6 mmol, 1.00 eq) in ethyl acetate (350 mL) was added tert-butyl (4S)-4,5-diamino-5-oxo-pentanoate hydrochloride (Compound 9 HCl, 43.22 g, 181.1 mmol, 1.025 eq), DMF (70 mL) and 2-MeTHF (110 mL) at 25° C. 2,6-Lutidine (23.4 mL, 201 mmol, 1.14 eq) was added slowly to maintain the temperature at or below 25° C. The mixture was aged at 25° C. for 1 hour before being cooled to 5° C. CDI (4.17 g, 25.7 mmol, 0.146 eq) was added and stirred until the temperature returned to 5° C. Another portion of CDI (4.62 g, 28.5 mmol, 0.161 eq) was added and stirred until temperature returned to 5° C. CDI (8.87 g, 54.7 mmol, 0.310 eq) was added and stirred until the temperature returned to 5° C. CDI (8.91 g, 54.9 mmol, 0.311 eq) was added and stirred until the temperature returned to 5° C. The mixture was warmed to 20° C. and CDI (16.4 g, 101.1 mmol, 0.573 eq) was added, and the mixture was aged at 20° C. for 16 hours. The mixture was cooled to 5° C. and a solution of 30 wt % citric acid and 5 wt % NaCl (350 mL) was added slowly while maintaining the temperature. The mixture was warmed to 20° C. and aged for 30 minutes. The phases were split and separated. The organic phase was diluted with EtOAc (175 mL) and washed with a solution of 5 wt % citric acid (175 mL), and concentrated by distillation (75 torr, 50° C.) to a volume of 175 mL EtOAc. The solvent was changed to iPrOH by constant volume distillation (75 torr, 50° C.) with 350 mL iPrOH to a final volume of 175 mL. The distillate was diluted with 200 mL iPrOH to afford Compound 6 as a solution for use in the next step. ¹H NMR (500 MHZ, CDCl₃) δ (ppm): 8.18-8.13 (m, 2H), 7.96 (t, J=7.8 Hz, 1H), 6.34 (s, 1H), 5.59 (s, 1H), 4.90 (dd, J=10.1, 4.6 Hz, 1H), 2.61 (ddt, J=14.6, 10.1, 6.1 Hz, 1H), 2.49 (ddt, J=14.2, 8.7, 5.2 Hz, 1H), 2.44-2.29 (m, 2H), 1.44 (s, 9H).

Synthesis of tert-butyl (S)-5-amino-4-(4-amino-1,3-dioxoisoindolin-2-yl)-5-oxopentanoate (Compound 5): To a solution of Compound 6 in iPrOH (375 mL) was added 5% palladium on carbon (1.23 g, 3.5 wt %, wet). The mixture was purged with nitrogen five times and with hydrogen three times. The mixture was pressurized with hydrogen (50 psi) and aged at 50° C. for 16 hours. The mixture was cooled to room temperature and purged with nitrogen three times, filtered to remove catalyst, and the filter cake was washed with iPrOH (20 mL) three times. The filtrate was concentrated to 200 mL, seeded (0.454 g, 1.3 wt %) at 22° C., and aged for 45 minutes. Water (1325 mL) was added over 3 hours at 22° C. After the addition of water, the mixture was cooled to 8° C. over 2 hours and aged for 1 hour at 8° C. The slurry was filtered, and the cake was rinsed with cold water (200 mL) three times and dried under vacuum at 50° C. to yield Compound 5 as a yellow solid (47.97 g, 80.6% yield, 99.62% LC purity, 103% ¹H NMR potency). ¹H NMR (500 MHZ, CDCl₃) δ (ppm): 7.46 (dd, J=8.3, 7.0 Hz, 1H), 7.19 (d, J=7.2 Hz, 1H), 6.89 (d, J=8.3 Hz, 1H), 6.28 (s, 1H), 5.41 (s, 1H), 5.28 (s, 2H), 4.83 (dd, J=9.3, 6.0 Hz, 1H), 2.52 (p, J=7.0 Hz, 2H), 2.36-2.29 (m, 2H), 1.44 (s, 9H). ¹³C NMR (126 MHz, CDCl₃) δ (ppm): 171.80, 171.12, 169.64, 168.27, 145.70, 135.50, 132.20, 121.43, 112.98, 80.99, 53.04, 32.23, 28.02, 24.36. LCMS (ESI): m/z 291.9 [M+H-tBu]

Synthesis of 4-(1-(4-bromo-3-fluorobenzyl)azetidin-3-yl)morpholine bis-methanesulfonic acid salt (Compound 4 bis-methanesulfonic acid salt): A mixture of 4-bromo-3-fluorobenzaldehyde (Compound 14, 102 g, 493 mmol) and 4-(azetidin-3-yl)morpholine hydrochloride (Compound 7 HCl, 90 g, 493 mmol) in acetonitrile (1000 ml) was agitated at a temperature of about 20 to 25° C. for 2 to 3 hours. The slurry was cooled to temperature of about 10 to 15° C. and sodium triacetoxyborohydride (STAB, 162 g, 739 mmol) was added in 4 portions over the course of about 45 minutes while maintaining the batch temperature at no more than 30° C. The slurry was stirred at a temperature of about 20 to 25° C. for at least 30 minutes and then quenched by an aqueous citric acid solution (191 g, 986 mmol in 500 ml of water) at a temperature of about 40 to 45° C. over the course of 2 hours. Upon completion of the quenching process, the batch volume was reduced by vacuum distillation to about 700 ml at a temperature of no more than 45° C. Cyclopentylmethylether (CPME, 400 ml) was added to the aqueous solution to afford a final volume of about 1100 ml. The pH was adjusted to about 8 to 9 by addition of an aqueous solution of 10 N NaOH (added volume about 430 ml). The phases were separated, and the aqueous phase discarded. The organic phase was washed with brine (100 ml) twice such that the pH was no more than 8 and the volume was adjusted to about 1000 ml with addition of extra CPME. The batch was distilled at constant volume under reduced pressure with addition of CPME until KF was no more than 0.15%. CPME was added (if needed) to adjust the batch to a volume of 1000 ml at the end of distillation. The dry CPME solution was seeded (500 to 750 mg) at ambient temperature. The seeded, dry CPME slurry was heated to a temperature of 50 to 60° C. and then charged with methanesulfonic acid in 200 ml of CPME over the course of 4 to 5 hours. The slurry was then cooled to 20° C. over the course of 4 to 5 hours and kept at 20° C. for 3 to 4 hours, filtered, rinsed with CPME and dried in a vacuum oven at 35 to 40° C. over 16 hours to give Compound 4 bis-methanesulfonic acid salt as a white solid. ¹H NMR (500 MHz DMSO-d₆) δ (ppm): 10.62 (br s, 1-2H), 7.85 (t, J=7.8 Hz, 1H), 7.58 (dd, J=9.5 Hz, 1.9 Hz, 1H), 7.34 (dd, J=8.2 Hz, 1.8 Hz, 1H), 4.55-4.24 (m, 7H), 3.84 (br s, 4H), 3.14 (m, 4H); HPLC purity, 99.8%, LCMS (ESI) m/z 329.1/331.1[M/M+2]⁺. XRPD pattern of the product is shown in FIG. 10. DSC thermogram of the product is shown in FIG. 11.

Preparation of 4-(1-(4-bromo-3-fluorobenzyl)azetidin-3-yl)morpholine (Compound 4): A slurry of Compound 4 bis-methanesulfonic acid salt (70 g, 134 mmol) in t-butyl methyl ether was cooled to 10±5° C. An aqueous solution of NaOH (2 N, 201 ml, 403 mmol) was added over the course of at least 30 minutes while maintaining the batch temperature at about 15° C. After the addition of NaOH, the batch temperature was raised to 20±5° C. and agitated over the course of about 20 minutes. The organic layer was separated and washed with water (210 ml) three times. The organic layer was subsequently concentrated with addition of THF (˜1.05 L) until KF≤0.10%. The product Compound 4 was isolated as a solution in THF with 95% solution yield.

Preparation of 2-fluoro-4-((3-morpholinoazetidin-1-yl)methyl)benzaldehyde dihydrochloride (Compound 3 di-HCl): A solution of Compound 4 (44 g, 134 mmol) in THF (total volume ˜350 ml) was then cooled to −20±5° C. A solution of iPrMgCl·LiCl (1.3 M, 176 ml, 228 mmol) in THF was added over the course of half an hour while maintaining the temperature below −10° C. After the addition was complete, the batch was stirred at −20±5° C. for 16 to 22 hours. DMF (21 ml, 268 mmol) was then added slowly over the course of 30 minutes while maintaining the batch temperature no more than −15° C. The batch was stirred at −20±5° C. for 6 to 24 hours. 2-MeTHF (350 ml) was then added to the batch over the course of 30 minutes, followed by slow addition of 3 N HCl (235 ml, 704 mmol) while keeping the batch temperature no more than −10° C. After the addition of aqueous HCl, the batch was warmed to 0±5° C. and 2 N aqueous NaOH (154 ml, 309 mmol) was added slowly to adjust the solution pH to about 8 to 9. The batch was stirred for about 30 minutes and then warmed to 20±5° C. The organic layer was separated and washed with 15% aqueous NaCl (3×140 ml). The organic layer was subsequently concentrated with addition of 2-MeTHF until KF≤0.10%.

A portion of the free base of 2-fluoro-4-((3-morpholinoazetidin-1-yl)methyl)benzaldehyde (37.4 g, 134 mmol) so obtained was dissolved in 2-MeTHF (total ˜420 ml), to which isopropanol (420 ml) and water (21 ml) were added at 20±5° C. The batch was then heated to 50±5° C. and a solution of HCl in IPA (5 to 6 N, 28 ml, half of total HCl volume) was added over the course of 1 hour. The batch was seeded with 2-fluoro-4-((3-morpholinoazetidin-1-yl)methyl)benzaldehyde dihydrochloride (700 mg) and aged for 1 hour. The remaining HCl (28 ml) was then added over the course of 1 hour. The batch was agitated at 50±5° C. for 4 hours and then cooled to 20±5° C. for 8 hours. The slurry was filtered, washed with IPA (210 ml), and the filter cake dried under vacuum at 50±5° C. to afford Compound 3 dihydrochloride salt (36 g, yield 75%). ¹H NMR (DMSO-d₆) δ (ppm): 12.32-12.55 (m, 1H), 10.23 (s, 1H), 7.93 (t, J=7.6 Hz, 1H), 7.66 (d, J=10.5 Hz, 1H), 7.58 (d, J=7.9 Hz, 1H), 4.80 (br s, 2H), 4.48-4.70 (m, 2H), 4.30 (br s, 4H), 3.78-4.00 (m, 5H), 2.93-3.15 (m, 2H). Two polymorphic forms were obtained. XRPD pattern and DSC thermogram of Form A (anhydrous) are shown in FIG. 5 and FIG. 6, respectively. XRPD pattern, TGA thermogram and DSC thermogram of Form B (hydrate) are shown in FIG. 7, FIG. 8, and FIG. 9, respectively.

Synthesis of tert-butyl (S)-5-amino-4-(4-((2-fluoro-4-((3-morpholinoazetidin-1-yl)methyl)benzyl)amino)-1,3-dioxoisoindolin-2-yl)-5-oxopentanoate (Compound 2): A mixture of Compound 5 (12 g, 34.5 mmol, 1.0 eq) and Compound 3 dihydrochloride (14.56 g, 41.5 mmol, 1.2 eq) in MeCN (96 ml) was cooled to 0-5° C. Trifluoroacetic acid (TFA, 2.0 ml, 26 mmol, 0.75 eq) was added, followed by sodium triacetoxyborohydride (STAB, 2.75 g, 12.95 mmol, 0.375 eq) while maintaining the internal temperature below 10° C. The addition of TFA and STAB was repeated three additional times. After a total of four additions of TFA and STAB, the reaction was aged at 0-5° C. for 1 hour. A 10% brine solution (108 ml) was then added to the reaction mixture over the course of 1 hour and partitioned with IPAc (96 ml). The mixture was warmed to 20-25° C. and aged for 30 minutes. The layers were then separated and the organic layer is washed with 2.0 M K₃PO₄ (114 ml). The pH of the spent aqueous layer should have a pH of about 8.5-9.0. The layers were separated again and the organic phase was washed with 8.5% NaHCO₃ (2×60 ml), with 30 minutes between each wash, followed by 24% brine (60 ml). The organic fraction was distilled to 72 ml at an internal temperature near 50° C. Toluene (72 ml) was added to bring the volume to 144 ml and distillation continued at constant volume at 50° C. with feed and bleed until water content <0.1. The mixture was heated to 50° C. and acetonitrile (48 ml) was added, followed by slow addition of heptane (144 ml) while maintaining the internal temperature above 45° C. The reaction was held at 50° C. for 2 hours. Once complete, the reaction was slowly cooled to 20-25° C. over the course of 4 hours and held at 20-25° C. overnight (16 hour). The yellow slurry was then filtered and the yellow cake displacement washed with 1:3:3 mixture of acetonitrile/heptane/toluene (3×48 ml). The final cake was then dried under reduced pressure at 50° C. under nitrogen to provide Compound 2 (87.7% isolated molar yield) with >99.0% LCAP. HPLC purity, 99.85%; Chiral purity, >99.9% ee. ¹H NMR (DMSO-d₆, 500 MHZ) δ (ppm) 7.55 (s, 1H), 7.51 (dd, J=7.2, 8.4 Hz, 1H), 7.32 (t, J=7.9 Hz, 1H), 7.16 (s, 1H), 7.0-7.1 (m, 5H), 4.57 (d, J=6.3 Hz, 2H), 4.5-4.5 (m, 1H), 3.5-3.6 (m, 6H), 3.3-3.4 (m, 3H), 2.8-2.9 (m, 3H), 2.3-2.4 (m, 1H), 2.1-2.3 (m, 7H), 1.31 (s, 9H); LCMS m/z 610.3 [M+H]⁺.

Synthesis of (S)-2-(2,6-dioxopiperidin-3-yl)-4-((2-fluoro-4-((3-morpholinoazetidin-1-yl)methyl)benzyl)amino)isoindoline-1,3-dione bis-besylate salt (Compound 1 bis-besylate): To a suspension of Compound 2 (130 g, 1.0 equiv.) in MeCN (1.56 L, 12 L/kg) agitated at 55° C. was added a solution of benzenesulfonic acid (185 g, 5.5 equiv.) in MeCN (0.39 L, 3 L/kg) and water (0.01 L, 2.0 equiv.). The mixture was stirred at 55° C. for 16 hours. After the reaction age, crystalline seeds (1.3 g, 1 wt %) of bis-besylate salt of Compound 1 were charged into the batch, resulting in formation of a yellow slurry. The slurry was then cooled to 20° C. over the course of 90 minutes. 2-MeTHF (1.3 L, 10 L/kg) was added to the batch slowly over 2 hours at 20° C. The batch was agitated for an additional 4 hours at 20° C. The yellow slurry was then filtered and the yellow cake re-slurried with MeTHF (1.3 L, 10 L/kg) followed by a displacement MeTHF (0.65 L, 5 L/kg) wash. The final cake was then dried under reduced pressure at 50° C. under nitrogen to give Compound 1 bis-besylate salt (160 g, 88.4% yield). HPLC purity: 98.39%; chiral HPLC purity: 100%. XRPD pattern, TGA thermogram and DSC thermogram of the product are shown in FIG. 1, FIG. 2, and FIG. 3, respectively.

Synthesis of (S)-2-(2,6-dioxopiperidin-3-yl)-4-((2-fluoro-4-((3-morpholinoazetidin-1-yl)methyl)benzyl)amino)isoindoline-1,3-dione hydrochloride salt (Compound 1 HCl): A suspension of Compound 1 bis-besylate salt (300 g, 1 equiv.) in EtOAc (4.68 L, 15.6 L/kg) and 2-propanol (0.12 L, 0.4 L/kg) was agitated at 15° C. To the suspension was added a solution of KHCO₃ (82.4 g, 2.5 equiv) in water (1.8 L, 6 L/kg) over 30 minutes. The mixture was heated to 20° C. over 30-60 minutes and then agitated for 30 minutes. The batch was allowed to settle for 30 minutes, at which point the aqueous (bottom) layer was discarded. To the rich organic layer was added water (1.2 L, 4 L/kg) and the reactor contents were agitated for 30 minutes. The batch was allowed to settle for 30 minutes, at which point the aqueous (bottom) layer was discarded. To the rich organic stream was added 2-propanol (2.375 L, 7.9 L/kg) and the stream was then filtered. Water was added to the filtrate to adjust the water content to 8≤KF<8.2. To the above agitated solution at 20° C. was added 0.2 N HCl (38 mL, 0.025 equiv prepared in EtOAC/IPA 2:1, v/v with 8 wt % water) over 10 minutes. To the mixture was added crystalline seeds of Compound 1 hydrochloride salt (1.6 g, 0.5 wt %) and the contents of the reactor were agitated at 20° C. for 30 minutes. To the suspension was added 0.2 N HCl (1.44 L, 0.945 equiv. prepared in EtOAC/IPA 2:1, v/v with 8 wt % water) over 4.5 hours. The slurry was agitated for 14 hours, then filtered and washed with EtOAC/IPA (750 mL, 2.5 L/kg, 2:1 v/v with 8 wt % water) followed by IPA (750 mL, 2.5 L/kg). The solids were dried under vacuum at 40° C. to afford Compound 1 hydrochloride salt (170 g, 90% yield). Achiral HPLC purity: 99.91%; chiral HPLC purity: 99.58%. XRPD analysis (FIG. 4) confirmed the product (a) as Form A of a hydrochloride salt of Compound 1 by comparison to a reference sample (b).

The embodiments described above are intended to be merely exemplary, and those skilled in the art will recognize, or will be able to ascertain using no more than routine experimentation, numerous equivalents of specific compounds, materials, and procedures. All such equivalents are considered to be within the scope of the claimed subject matter and are encompassed by the appended claims.

All of the patents, patent applications and publications referred to herein are incorporated herein in their entireties. Citation or identification of any reference in this application is not an admission that such reference is available as prior art to the claimed subject matter.

What is claimed is:

1. A process for preparing a compound of Formula (I): 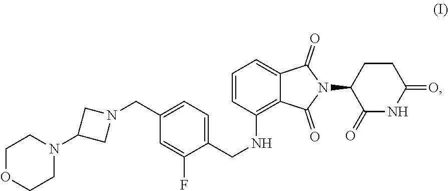 [SMILES: O=C1CC[C@H](N2C(=O)c3cccc(NCc4ccc(CN5CC(N6CCOCC6)C5)cc4F)c3C2=O)C(=O)N1]
or a salt, solvate, hydrate, enantiomer, mixture of enantiomers, or isotopologue thereof, comprising:
(step 1.0) cyclizing a compound of Formula (II): 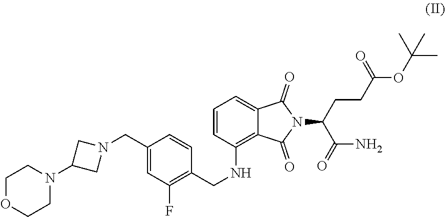 [SMILES: CC(C)(C)OC(=O)CC[C@@H](C(N)=O)N1C(=O)c2cccc(NCc3ccc(CN4CC(N5CCOCC5)C4)cc3F)c2C1=O]
or a salt, solvate, hydrate, enantiomer, mixture of enantiomers, or isotopologue thereof, to provide a compound of Formula (I), or a salt, solvate, hydrate, enantiomer, mixture of enantiomers, or isotopologue thereof; and
(step 1.1) optionally converting the compound of Formula (I), or a salt, solvate, hydrate, enantiomer, mixture of enantiomers, or isotopologue thereof, to a salt of the compound.

2. The process of claim 1, wherein step 1.0 occurs in the presence of an acid.

3. The process of claim 2, wherein the acid is benzenesulfonic acid.

4. The process of claim 3, wherein the compound of Formula (I), or a salt, solvate, hydrate, enantiomer, mixture of enantiomers, or isotopologue thereof, prepared in step 1.0 is a besylate salt.

5. The process of any one of claims 1 to 4, wherein step 1.0 occurs in a solvent of acetonitrile, methyltetrahydrofuran, water, or a combination thereof.

6. The process of any one of claims 1 to 5, wherein in step 1.1 the compound of Formula (I), or a salt, solvate, hydrate, enantiomer, mixture of enantiomers, or isotopologue thereof, is converted to a hydrochloride salt of the compound.

7. The process of claim 6, wherein in step 1.1 a salt of the compound of Formula (I) is contacted with a basic aqueous solution and is subsequently acidified.

8. The process of claim 7, wherein the basic aqueous solution is a bicarbonate solution.

9. The process of claim 7 or 8, wherein acidification comprises addition of hydrochloric acid.

10. The process of any one of claims 6 to 9, wherein step 1.1 occurs in a biphasic mixture comprising an aqueous solution and an organic solvent.

11. The process of any one of claims 6 to 10, wherein step 1.1 occurs in a solvent of ethyl acetate (EtOAc), isopropanol (IPA), or water.

12. The process of any one of claims 1 to 11, wherein the compound of Formula (II), or a salt, solvate, hydrate, enantiomer, mixture of enantiomers, or isotopologue thereof, is prepared by a process comprising:
(step 2.a) reacting a compound of Formula (II-A): 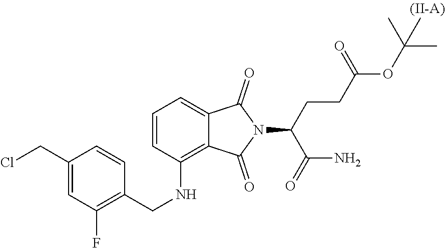 [SMILES: CC(C)(C)OC(=O)CC[C@@H](C(N)=O)N1C(=O)c2cccc(NCc3ccc(CCl)cc3F)c2C1=O]
or a salt, solvate, hydrate, enantiomer, mixture of enantiomers, or isotopologue thereof, with 4-(azetidin-3-yl)morpholine, or a salt thereof.

13. The process of claim 12, wherein step 2.a occurs in the presence of a base.

14. The process of claim 13, wherein the base is diisopropylethylamine (DIEA).

15. The process of any one of claims 12 to 14, wherein the compound of Formula (II-A), or a salt, solvate, hydrate, enantiomer, mixture of enantiomers, or isotopologue thereof, is prepared by a process comprising:
(step 2.b) chlorinating a compound of Formula (II-B): 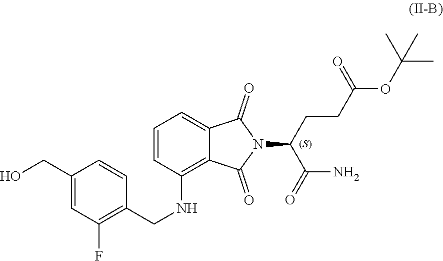
or a salt, solvate, hydrate, enantiomer, mixture of enantiomers, or isotopologue thereof.

16. The process of claim 15, wherein chlorination in step 2.b occurs in the presence of mesyl chloride (MsCl).

17. The process of claim 15 or 16, wherein step 2.b occurs in the presence of base.

18. The process of claim 17, wherein the base is diisopropylethylamine (DIEA).

19. The process of any one of claims 15 to 18, wherein the compound of Formula (II-B), or a salt, solvate, hydrate, enantiomer, mixture of enantiomers, or isotopologue thereof, is prepared by a process comprising:
(step 2.c) reacting a compound of Formula (V): 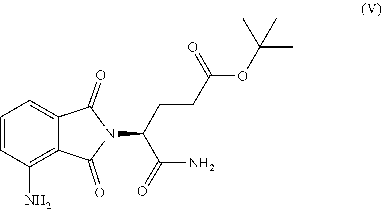 [SMILES: CC(C)(C)OC(=O)CC[C@@H](C(N)=O)N1C(=O)c2cccc(N)c2C1=O]
or a salt, solvate, hydrate, enantiomer, mixture of enantiomers, or isotopologue thereof, with 2-fluoro-4-(hydroxymethyl)benzaldehyde.

20. The process of claim 19, wherein step 2.c occurs in the presence of a reducing agent.

21. The process of claim 20, wherein the reducing agent is a borohydride reagent.

22. The process of claim 21, wherein the borohydride reagent is sodium cyanoborohydride.

23. The process of any one of claims 19 to 22, wherein step 2.c occurs in the presence of acid.

24. The process of claim 23, where in the acid is trifluoroacetic acid.

25. The process of any one of claims 1 to 11, wherein the compound of Formula (II), or a salt, solvate, hydrate, enantiomer, mixture of enantiomers, or isotopologue thereof, is prepared by a process comprising:
(step 2.0) reacting a compound of Formula (III): 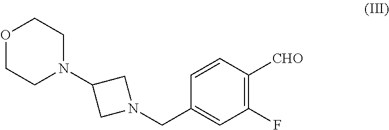 [SMILES: O=Cc1ccc(CN2CC(N3CCOCC3)C2)cc1F]
or a salt, solvate, hydrate, or isotopologue thereof, with a compound of Formula (V): 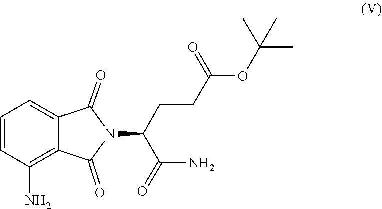 [SMILES: CC(C)(C)OC(=O)CC[C@@H](C(N)=O)N1C(=O)c2cccc(N)c2C1=O]
or a salt, solvate, hydrate, enantiomer, mixture of enantiomers, or isotopologue thereof.

26. The process of claim 25, wherein in step 2.0 a bis-hydrochloride salt of the compound of Formula (III) is used.

27. The process of claim 25, wherein in step 2.0 a bis-oxalic acid salt of the compound of Formula (III) is used.

28. The process of any one of claims 25 to 27, wherein step 2.0 occurs in the presence of a reducing agent.

29. The process of claim 28, wherein the reducing agent is a borohydride reagent.

30. The process of claim 29, wherein the borohydride reagent is sodium triacetoxyborohydride.

31. The process of any one of claims 25 to 30, wherein step 2.0 occurs in the presence of acid.

32. The process of claim 31, wherein the acid is trifluoroacetic acid.

33. The process of any one of claims 25 to 32, wherein the compound of Formula (III), or a salt, solvate, hydrate, or isotopologue thereof, is prepared by a process comprising:
(step 3.0) reacting a compound of Formula (IV): 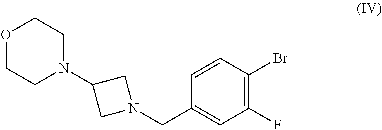 [SMILES: Fc1cc(CN2CC(N3CCOCC3)C2)ccc1Br]
or a salt, solvate, hydrate, or isotopologue thereof, with a formaldehyde source.

34. The process of claim 33, wherein a salt of a compound of Formula (IV) is converted to the free base form of a compound of Formula (IV), which is then used in step 3.0.

35. The process of claim 34, wherein the free base form of a compound of Formula (IV) is formed by contacting a salt of a compound of Formula (IV) with a basic aqueous solution and, optionally, an organic solvent.

36. The process of claim 35, wherein the organic solvent is methyl tert-butyl ether (MTBE).

37. The process of any one of claims 33 to 36, wherein the salt of a compound of Formula (IV) is a methanesulfonic acid salt.

38. The process of any one of claims 33 to 37, wherein the formaldehyde source is dimethylformamide (DMF).

39. The process of any one of claims 33 to 38, wherein step 3.0 occurs in the presence of an organomagnesium reagent.

40. The process of claim 39, where in the organomagnesium reagent is iPrMgCl·LiCl.

41. The process of any one of claims 33 to 40, wherein step 3.0 occurs in a solvent comprising tetrahydrofuran (THF), methyl tert-butyl ether (MTBE), or dimethylformamide (DMF), or a mixture thereof.

42. The process of any one of claims 33 to 41, wherein the reaction temperature for step 3.0 is from about −30 to about 10° C.

43. The process of any one of claims 33 to 42, wherein in step 3.0 the compound of Formula (III), or a salt, solvate, hydrate, enantiomer, mixture of enantiomers, or isotopologue thereof, is converted to a bis-hydrochloride salt of the compound.

44. The process of any one of claims 25 to 32, further comprising:
(step 3.a) reacting the compound of Formula (III), or a salt, solvate, hydrate, or isotopologue thereof, prepared in step 3.0 with Na₂S₂O₅ to provide a sodium sulfonate compound of the Formula: 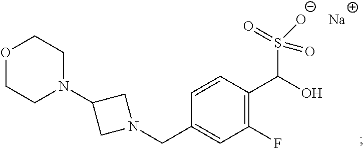 [SMILES: O=S(=O)([O-])C(O)c1ccc(CN2CC(N3CCOCC3)C2)cc1F ; [Na+]]
or a salt, solvate, hydrate, or isotopologue thereof, and
(step 3.b) converting the sodium sulfonate compound to the compound of Formula (III), or a salt, solvate, hydrate, or isotopologue thereof.

45. The process of claim 44, wherein step 3.a occurs in a mixed solvent of ethanol and water.

46. The process of claim 44 or 45, wherein step 3.b occurs in the presence of base.

47. The process of claim 46, wherein the base is potassium carbonate.

48. The process of any one of claims 44 to 47, wherein in step 3.b the compound of Formula (III), or a salt, solvate, hydrate, enantiomer, mixture of enantiomers, or isotopologue thereof, is converted to a bis-oxalic acid salt of the compound.

49. The process of any one of claims 33 to 48, wherein the compound of Formula (IV), or a salt, solvate, hydrate, or isotopologue thereof, is prepared by a process comprising:
(step 4.0) reacting 4-(azetidin-3-yl)morpholine, or a salt thereof, with 4-bromo-3-fluorobenzaldehyde.

50. The process of claim 49, wherein in step 4.0 a hydrochloride salt of 4-(azetidin-3-yl)morpholine is used.

51. The process of claim 49 or 50, wherein step 4.0 occurs in the presence of a reducing agent.

52. The process of claim 51, wherein the reducing agent is a borohydride reagent.

53. The process of claim 52, wherein the borohydride reagent is sodium triacetoxyborohydride.

54. The process of any one of claims 49 to 53, wherein in step 4.0 the compound of Formula (IV), or a salt, solvate, hydrate, enantiomer, mixture of enantiomers, or isotopologue thereof, is converted to a methanesulfonic acid salt of the compound.

55. The process of any one of claims 49 to 54, wherein step 4.0 occurs in a solvent of acetonitrile, cyclopentyl methyl ether (CPME), or methanol.

56. The process of any one of claims 25 to 32, wherein the compound of Formula (V), or a salt, solvate, hydrate, enantiomer, mixture of enantiomers, or isotopologue thereof, is prepared by a process comprising:
(step 5.0) reducing a compound of Formula (VI): 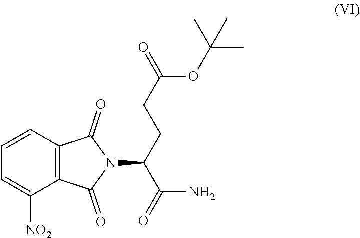 [SMILES: CC(C)(C)OC(=O)CC[C@@H](C(N)=O)N1C(=O)c2cccc([N+](=O)[O-])c2C1=O]
or a salt, solvate, hydrate, enantiomer, mixture of enantiomers, or isotopologue thereof.

57. The process of claim 56, wherein step 5.0 occurs by hydrogenation.

58. The process of claim 57, wherein hydrogenation is accomplished using hydrogen gas.

59. The process of any one of claims 56 to 58, wherein step 5.0 occurs in the presence of a catalyst.

60. The process of claim 59, wherein the catalyst is palladium on carbon.

61. The process of any one of claims 56 to 60, wherein the compound of Formula (VI), or a salt, solvate, hydrate, enantiomer, mixture of enantiomers, or isotopologue thereof, is prepared by a process comprising:
(step 6.0) reacting (S)-tert-butyl 4,5-diamino-5-oxopentanoate of the Formula: 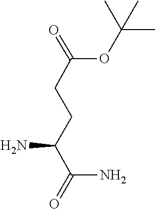
or a salt, solvate, hydrate, enantiomer, mixture of enantiomers, or isotopologue thereof, with 3-nitrophthalic anhydride.

62. The process of claim 61, wherein step 6.0 occurs in the presence of base.

63. The process of claim 62, wherein the base is lutidine.

64. The process of any one of claims 61 to 63, wherein step 6.0 occurs in the presence of an activating reagent.

65. The process of claim 64, wherein the activating reagent is 1,1′-carbonyldiimidazole.

66. The process of any one of claims 56 to 60, wherein the compound of Formula (VI), or a salt, solvate, hydrate, enantiomer, mixture of enantiomers, or isotopologue thereof, is prepared by a process comprising:
(step 6.a) reacting (S)-tert-butyl 4,5-diamino-5-oxopentanoate of the Formula: 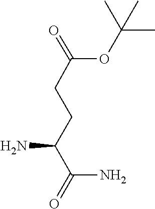
or a salt, solvate, hydrate, enantiomer, mixture of enantiomers, or isotopologue thereof, with ethyl 4-nitro-1,3-dioxoisoindoline-2-carboxylate of the Formula:

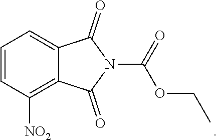 [SMILES: CCOC(=O)N1C(=O)c2cccc([N+](=O)[O-])c2C1=O]

67. The process of claim 66, wherein step 6.a occurs in the presence of base.

68. The process of claim 67, wherein the base is diisopropylethylamine (DIEA).

69. The process of any one of claims 66 to 68, wherein ethyl 4-nitro-1,3-dioxoisoindoline-2-carboxylate is prepared by a process comprising:
(step 6.b) reacting 4-nitroisoindoline-1,3-dione with ethyl chloroformate.

70. The process of claim 69, wherein step 6.b occurs in the presence of base.

71. The process of claim 70, wherein the base is trimethylamine (TEA).

72. The process of claim 1, wherein a compound of Formula I, or a salt, solvate, hydrate, enantiomer, mixture of enantiomers, or isotopologue thereof, is prepared by a process comprising:
(step 1.0) cyclizing a compound of Formula (II), or a salt, solvate, hydrate, enantiomer, mixture of enantiomers, or isotopologue thereof, to provide a compound of Formula (I), or a salt, solvate, hydrate, enantiomer, mixture of enantiomers, or isotopologue thereof; and
(step 1.1) optionally converting the compound of Formula (I), or a salt, solvate, hydrate, enantiomer, mixture of enantiomers, or isotopologue thereof, to a salt of the compound;
wherein the compound of Formula (II), or a salt, solvate, hydrate, enantiomer, mixture of enantiomers, or isotopologue thereof, is prepared by a process comprising:
(step 2.0) reacting a compound of Formula (III), or a salt, solvate, hydrate, or isotopologue thereof, with a compound of Formula (V) or a salt, solvate, hydrate, enantiomer, mixture of enantiomers, or isotopologue thereof;
wherein the compound of Formula (III), or a salt, solvate, hydrate, or isotopologue thereof, is prepared by a process comprising:
(step 3.0) reacting a compound of Formula (IV), or a salt, solvate, hydrate, or isotopologue thereof, with a formaldehyde source;
wherein the compound of Formula (IV), or a salt, solvate, hydrate, or isotopologue thereof, is prepared by a process comprising:
(step 4.0) reacting 4-(azetidin-3-yl)morpholine, or a salt thereof, with 4-bromo-3-fluorobenzaldehyde;
wherein the compound of Formula (V), or a salt, solvate, hydrate, enantiomer, mixture of enantiomers, or isotopologue thereof, is prepared by a process comprising:
(step 5.0) reducing a compound of Formula (VI), or a salt, solvate, hydrate, enantiomer, mixture of enantiomers, or isotopologue thereof; and
wherein the compound of Formula (VI), or a salt, solvate, hydrate, enantiomer, mixture of enantiomers, or isotopologue thereof, is prepared by a process comprising:
(step 6.0) reacting (S)-tert-butyl 4,5-diamino-5-oxopentanoate or a salt, solvate, hydrate, enantiomer, mixture of enantiomers, or isotopologue thereof, with 3-nitrophthalic anhydride.

73. A bis-besylate salt of Compound 1.

74. A compound, which is Compound 2, Compound 2-a, Compound 2-b, Compound 3, Compound 4, Compound 5, or Compound 6, or a salt, solvate, hydrate, enantiomer, mixture of enantiomers, or isotopologue thereof.

75. A solid form comprising a besylate salt of Compound 1: 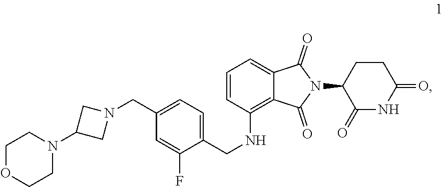 [SMILES: O=C1CC[C@H](N2C(=O)c3cccc(NCc4ccc(CN5CC(N6CCOCC6)C5)cc4F)c3C2=O)C(=O)N1]
wherein the solid form is Form B of a besylate salt of Compound 1.

76. The solid form of claim 75, which is characterized by an XRPD pattern comprising peaks at approximately 6.7, 7.5, and 17.2° 2θ.

77. The solid form of claim 76, wherein the XRPD pattern further comprises peaks at approximately 16.0 and 23.5° 2θ.

78. The solid form of claim 77, wherein the XRPD pattern further comprises peaks at approximately 9.4 and 11.3° 2θ.

79. The solid form of claim 75, which is characterized by an XRPD pattern that matches the XRPD pattern presented in FIG. 1.

80. A solid form comprising a hydrochloride salt of Compound 3:

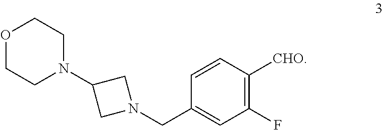 [SMILES: O=Cc1ccc(CN2CC(N3CCOCC3)C2)cc1F]

81. The solid form of claim 80, which is Form A of a hydrochloride salt of Compound 3, characterized by an XRPD pattern comprising peaks at approximately 14.6, 19.4, and 21.8° 2θ. 2θ.

82. The solid form of claim 81, wherein the XRPD pattern further comprises peaks at approximately 15.8 and 22.8° 2θ.

83. The solid form of claim 82, wherein the XRPD pattern further comprises peaks at approximately 8.8, 14.3, and 14.9° 2θ.

84. The solid form of claim 81, which is characterized by an XRPD pattern that matches the XRPD pattern presented in FIG. 5.

85. The solid form of claim 80, which is Form B of a hydrochloride salt of Compound 3, characterized by an XRPD pattern comprising peaks at approximately 14.3, 15.4, and 16.2° 2θ.

86. The solid form of claim 85, wherein the XRPD pattern further comprises peaks at approximately 14.8, 17.8, and 19.4° 2θ.

87. The solid form of claim 86, wherein the XRPD pattern further comprises peaks at approximately 7.8 and 21.0° 2θ.

88. The solid form of claim 85, which is characterized by an XRPD pattern that matches the XRPD pattern presented in FIG. 7.

89. A solid form comprising a methanesulfonic acid salt of Compound 4:

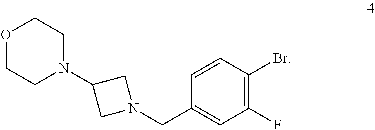 [SMILES: Fc1cc(CN2CC(N3CCOCC3)C2)ccc1Br]

90. The solid form of claim 89, which is Form A of a methanesulfonic acid salt of Compound 4, characterized by an XRPD pattern comprising peaks at approximately 18.6, 20.3, and 20.8° 2θ.

91. The solid form of claim 90, wherein the XRPD pattern further comprises peaks at approximately 16.7 and 22.7° 2θ.

92. The solid form of claim 91, wherein the XRPD pattern further comprises peaks at approximately 8.0 and 24.6° 2θ.

93. The solid form of claim 90, which is characterized by an XRPD pattern that matches the XRPD pattern presented in FIG. 10.
